SEQUENCE LISTING (73 sequences)

SEQ ID NO: 1 - DNA, 5050 bp, synthetic construct
  misc_feature 1..5050: note = ERG20-PGAL-tHMGR insert of pAM489
  source 1..5050: mol_type = other DNA
gtttaaactactattagctgaattgccactgctatcgttgttagtggcgttagtgcttgcattcaaagacatggagggcgttattacgccggagctcctcgacagcagatctgatgactggtcaatatatttttgcattgaggctctgtttggaattatattttgagatgacccatctaatgtactggtatcaccagatttcatgtcgttttttaaagcggctgcttgagtcttagcaatagcgtcaccatctggtgaatcctttgaaggaaccactgacgaaggtttggacagtgacgaagaggatctttcctgctttgaattagtcgcgctgggagcagatgacgagttggtggagctgggggcaggattgctggccgtcgtgggtcctgaatgggtccttggctggtccatctctattctgaaaacggaagaggagtagggaatattactggctgaaaataagtcttgaatgaacgtatacgcgtatatttctaccaatctctcaacactgagtaatggtagttataagaaagagaccgagttagggacagttagaggcggtggagatattccttatggcatgtctggcgatgataaaacttttcaaacggcagccccgatctaaaagagctgacacccgggttaaaaaaaatccttggactagtcacgtggaacggtagatcgagagatactatcttaaagcttatactatattatattggaagtatatagttcttatacatggtccttatctagtttgaaatctaatgttttatcgattagcgttagttctatttgcttctcttgtaaactttgttcaagaacgcagttaagacatcagctttgaagccacgagactcatcgacctgagaaattttggccttcaaatccttggcaatagactcttcatattcgtggtatagctgttcaattttcaagtcattgaaaatctttttgcatttggcttctgcgactgagtccttcttaccgtaattttcgtctaaagtctttctttgttctgcggaagcaagttccaatgccttgttgattacccaagaacatttgttatcttggatatctgtaccgatcttaccgatctgttctggggtaccgaagcagtctaagtagtcatcttgaatttggaagtattcacccaatggaatcaagacatctctggcttgtttcaaatccttttcatccgtgataccggcaacgtacatggccaatgcgacaggcaagtagaaagaatagtaagcagtcttgaaagtaactatgaaggagtgcttctttagggagaacttactcaagtcgactttgtcttcaggtgcagtgattaagtccatcaattggcccaattcggtttggaaggtgacctcatggaacaattcggtgatatctatgtagtatttttcgtttctgaagtgagatttcaaaagcttgtagatagcagcctctaacatgaatgcgtcattgatggcaatttccccaacttcaggaaccttgtaccaacatggttggcctcttctggtaatggacttgtccatcatatcatcggcgaccaagaagtaagcctgcaacaactcaatgcaccaacctagaatggcaaccttttcgtattcttcttgccccaattgttcaacggtcttgttggagagaatagcatacgtgtccacaacggacaaacctctatttagcttaccgcctggagtgttgtagttcaatgagtgggcataccagtcacatgcttccttaggcataccgtaagccaaaagcgatgcgttcaattcctctactaatttagggaaaacgttcaagaatctctctctcctaatttctttttctgaagccatttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactataatggctgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaaacttagtcatacgtcattggtattctcttgaaaaagaagcacaacagcaccatgtgttacgtaaaatatttactttatagtttgtacgtcataatttcttccatattacaagttcgtgcatatatagaaagaattctgttgttgtaattgtcataactcccgggatatatgtgtactttgcagttatgacgccagatggcagtagtggaagatattctttattgaaaaatagcttgtcaccttacgtacaatcttgatccggagcttttctttttttgccgattaagaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccgtttaaac

SEQ ID NO: 2 - DNA, 5488 bp, synthetic construct
  misc_feature 1..5488: note = ERG13-PGAL-tHMGR insert of pAM491
  source 1..5488: mol_type = other DNA
gtttaaacttgctaaattcgagtgaaacacaggaagaccagaaaatcctcatttcatccatattaacaataatttcaaatgtttatttgcattatttgaaactagggaagacaagcaacgaaacgtttttgaaaattttgagtattttcaataaatttgtagaggactcagatattgaaaaaaagctacagcaattaatacttgataagaagagtattgagaagggcaacggttcatcatctcatggatctgcacatgaacaaacaccagagtcaaacgacgttgaaattgaggctactgcgccaattgatgacaatacagacgatgataacaaaccgaagttatctgatgtagaaaaggattaaagatgctaagagatagtgatgatatttcataaataatgtaattctatatatgttaattaccttttttgcgaggcatatttatggtgaaggataagttttgaccatcaaagaaggttaatgtggctgtggtttcagggtccatacccgggtatatatatatcattgttattaaataaagagttttcctagtatatagattaaaaaactactctattaaatgagagctaaaaaaagcaggctgccaaaaaaataaagcatttatgaagggggttcagcaagatgcaatcgatgggggaagattattttttaacatcgtaagatcttctaaatttgtcatcgatgttggtcaagtagtaaacaccactttgcaaatgctcaatggaaccttgaggtttgaagttcttcttcaaatgggcattttctctcaattcgatggcagcttcgtaatcctttggagtttcggtgattctcttggctaatttgttagtaatatctaattccttgataatatgttggacgtcaccaacaattttgcaagaatatagagatgcagctaaaccggaaccgtaagaaaataaaccaacacgcttgccttgtaagtcgtcagatccaacatagtttaatagagatgcaaaggcggcataaacagatgcggtgtacatgttacctgtgtttgttggaacaatcaaagattgggcaactctctctttgtggaatggcttagcaacattaacaaaagttttttcaatgttcttatcggttaaagattcgtcataatcgcgagtagctaattcggcgtcaacttctgggaacaattgaggattggctctgaaatcgttatatagtaatctaccgtatgattttgtgaccaatttacaggttggaacatggaaaacgttgtagtcgaaatatttcaaaacgttcaaagcatccgaaccagcgggatcgctaaccaaccctttagaaatagccttcttggaataactcttgtaaacttgatcaagagccttgacgtaacaagttaatgaaaaatgaccatcgacgtaaggatattcgctggtgaaatctggcttgtaaaaatcgtaggcgtgttccatgtaagaagctcttacagagtcaaatacaattggagcatcaggaccgatccacatagcaacagtaccggcaccaccggttggtcttgcggcacccttatcgtagatggcaatatcaccgcaaactacaatggcgtctctaccatcccatgcgttagattcaatccagttcaaagagttgaacaacgcgttggtaccaccgtaacaggcattaagcgtgtcaataccttcgacgtcagtgttttcaccaaacaattgcatcaagacagacttgacagacttggacttgtcaatcagagtttcagtaccgacttctaatctaccaattttgttggtgtcgatgttgtaactcttgatcaacttagacaaaacagttagggacatcgagtagatatcttctctgtcattgacaaaagacatgttggtttggcccagaccaattgtgtatttaccttgagaaacgccatcaaatttctctagctcagattggttgacacattgagttgggatgtaaatttggatacctttaataccgacattttgaggtctggttttttgttcagcggtcttttgtttttttagttcagtcatttgcaagtttgtattgtgtaattgttgttgcttttgcggcctaagtcttcctttaataccacaccaacaaagtttagttgagagtttcatttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactataatggctgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaaacttagtcatacgtcattggtattctcttgaaaaagaagcacaacagcaccatgtgttacgtaaaatatttactttatagtttgtacgtcataatttcttccatattacaagttcgtgcatatatagaaagaattctgttgttgtaattgtcataactcccgggaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttgggtttaaac

SEQ ID NO: 3 - DNA, 4933 bp, synthetic construct
  misc_feature 1..4933: note = IDI1-PGAL-tHMGR insert of pAM493
  source 1..4933: mol_type = other DNA
gtttaaactactcagtatattaagtttcgaattgaagggcgaactcttattcgaagtcggagtcaccacaacacttccgcccatactctccgaatcctcgtttcctaaagtaagtttacttccacttgtaggcctattattaatgatatctgaataatcctctattagggttggatcattcagtagcgcgtgcgattgaaaggagtccatgcccgacgtcgacgtgattagcgaaggcgcgtaaccattgtcatgtctagcagctatagaactaacctccttgacaccacttgcggaagtctcatcaacatgctcttccttattactcattctcttaccaagcagagaatgttatctaaaaactacgtgtatttcacctctttctcgacttgaacacgtccaactccttaagtactaccacagccaggaaagaatggatccagttctacacgatagcaaagcagaaaacacaaccagcgtacccctgtagaagcttctttgtttacagcacttgatccatgtagccatactcgaaatttcaactcatctgaaacttttcctgaaggttgaaaaagaatgccataagggtcacccgaagcttattcacgcccgggtaaataaagaaaataaagtttacatattactaaggatttttgtcgcctatttttactatttttcaggtgaaatgaaacgttttatatcacattttgctatgataacaaagttattatgatttttatgtagcctatattattgacgcgttgttatagcattctatgaatttgcctgtcattttccacttcagaaaggtcatctaattgctcccaccagttgaataagtaattctcgcaaataatcttaaaccaaggcgtaaacttgtaacttgggtcagcaaacatagttttcaaatcatttggtgaaacccatttgaagtctctaacttcattgacgtttgggttgacagtcaagttttctttagcgttgatcttataaaataggatgtaatcaatttcatgttcaccccatggttcattgcttggtgccatgtaatggattctgtttaaaaagtgaaacttaccccttgtcttagtttcatcttctggaatacctaattcatgatctagttttctcaccgccgcagtaatagcgcccttaatcttatcgtctagcttacccttcaaacctaattcgtcatcaatacatagtggatgagagcagcatgtgttagtccaaagatcagggaaagttattttttcagtggctctttgttgtaaaagtaattcaccttgttcattgaaaataaagacggagaatgcacgatgtagtaaacccttttcaatattttccattaaatgacaaactttcttggtaccggcaccaatagcattatcgtcccaatccaaaacaatacaattttcattcattaacttaatttgctcctcatcatgaccagaaaaacatgtttctccgctttcgtcatttgacgtctcactagatcgggtattaggtctttgttgtaatggaataatttcaggaaactcttccaaaatgtcttcaggtgtttggttttgcactaatttggcgtaactagatactgcaccatggggcatactattgttgtcggcagtcatttatattgaattttcaaaaattcttactttttttttggatggacgcaaagaagtttaataatcatattacatggcattaccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactataatggctgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaaacttagtcatacgtcattggtattctcttgaaaaagaagcacaacagcaccatgtgttacgtaaaatatttactttatagtttgtacgtcataatttcttccatattacaagttcgtgcatatatagaaagaattctgttgttgtaattgtcataactcccgggagtcagtctgactcttgcgagagatgaggatgtaataatactaatctcgaagatgccatctaatacatatagacatacatatatatatatatacattctatatattcttacccagattctttgaggtaagacggttgggttttatcttttgcagttggtactattaagaacaatcgaatcataagcattgcttacaaagaatacacatacgaaatattaacgataatgtcaattacgaagactgaactggacggtatattgccattggtggccagaggtaaagttagagacatatatgaggtagacgctggtacgttgctgtttgttgctacggatcgtatctctgcatatgacgttattatggaaaacagcattcctgaaaaggggatcctattgaccaaactgtcagagttctggttcaagttcctgtccaacgatgttcgtaatcatttggtcgacatcgccccaggtaagactattttcgattatctacctgcaaaattgagcgaaccaaagtacaaaacgcaactagaagaccgctctctattggttcacaaacataaactaattccattggaagtaattgtcagaggctacatcaccggatctgcttggaaagagtacgtaaaaacaggtactgtgcatggtttgaaacaacctcaaggacttaaagaatctcaagagttcccagaaccaatcttcaccccatcgaccaaggctgaacaaggtgaacatgacgaaaacatctctcctgcccaggccgctgagctggtgggtgaagatttgtcacgtagagtggcagaactggctgtaaaactgtactccaagtgcaaagattatgctaaggagaagggcatcatcatcgcagacactaaattgtttaaac

SEQ ID NO: 4 - DNA, 6408 bp, synthetic construct
  misc_feature 1..6408: note = ERG10-PGAL-ERG12 insert of pAM495
  source 1..6408: mol_type = other DNA
gtttaaactattgtgagggtcagttatttcatccagatataacccgagaggaaacttcttagcgtctgttttcgtaccataaggcagttcatgaggtatattttcgttattgaagcccagctcgtgaatgcttaatgctgctgaactggtgtccatgtcgcctaggtacgcaatctccacaggctgcaaaggttttgtctcaagagcaatgttattgtgcaccccgtaattggtcaacaagtttaatctgtgcttgtccaccagctctgtcgtaaccttcagttcatcgactatctgaagaaatttactaggaatagtgccatggtacagcaaccgagaatggcaatttctactcgggttcagcaacgctgcataaacgctgttggtgccgtagacatattcgaagataggattatcattcataagtttcagagcaatgtccttattctggaacttggatttatggctcttttggtttaatttcgcctgattcttgatctcctttagcttctcgacgtgggcctttttcttgccatatggatccgctgcacggtcctgttccctagcatgtacgtgagcgtatttccttttaaaccacgacgctttgtcttcattcaacgtttcccattgtttttttctactattgctttgctgtgggaaaaacttatcgaaagatgacgactttttcttaattctcgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcattttttttttttccacctagcggatgactctttttttttcttagcgattggcattatcacataatgaattatacattatataaagtaatgtgatttcttcgaagaatatactaaagtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatggcagaaccagcccaaaaaaagcaaaaacaaactgttcaggagcgcaaggcgtttatctcccgtatcactaatgaaactaaaattcaaatcgctatttcgctgaatggtggttatattcaaataaaagattcgattcttcctgcaaagaaggatgacgatgtagcttcccaagctactcagtcacaggtcatcgatattcacacaggtgttggctttttggatcatatgatccatgcgttggcaaaacactctggttggtctcttattgttgaatgtattggtgacctgcacattgacgatcaccatactaccgaagattgcggtatcgcattagggcaagcgttcaaagaagcaatgggtgctgtccgtggtgtaaaaagattcggtactgggttcgcaccattggatgaggcgctatcacgtgccgtagtcgatttatctagtagaccatttgctgtaatcgaccttggattgaagagagagatgattggtgatttatccactgaaatgattccacactttttggaaagtttcgcggaggcggccagaattactttgcatgttgattgtctgagaggtttcaacgatcaccacagaagtgagagtgcgttcaaggctttggctgttgccataagagaagctatttctagcaatggcaccaatgacgttccctcaaccaaaggtgttttgatgtgaagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccacccgggaaaagtaagtcaaaaggcacacctcagcgtttgagtacctgaaaaacgatgaatcgcaaataaaactttaaattatgcctgttatacataaagccatttatatatttatgtattttatgaaaaagatcatgagaaaatcgcagaacgtaatcatatcttttcaatgacaatagaggaagcaccaccaccaccattacaaatggcggcaacaccgatcttacctccttcttgctgtaagatggatagcagtgtaacaaccactctagcaccagaacaacccaatgggtgacctagagcaacagcaccaccatatacattaaccttagatgggtctagcttcaaaatcttagtgttcaccaaaccgacaaccgaaaaggcttcattgaattcaaagtaatcaacagaattgatgtcttcgatgccagcatgtttcaaagcctttggaactgcaagagatggagcccatgtaaaatcagctggttgatgagcggcctcaccccaacctttgataatagccaaaggcttcaaattcttttccttcaaaactttttcggaaaccaagatgacggctgcagcaccatcgttgattggagaagcgttagcggcagtaacagtaccgttttctttttggaaaacagtccttgcagatctcaatttttcaacgtgtaatctagcaggttcctcgtccttcgtgacttgagtatcaggcttacctctaaatcccttaatggtaacaggtacaatttcattgtcgaatttaccttccttttgagatttttgagatttttggtaggattcgatggcaaaattgtcttgttgttctctagtaatatcccaatcacgggcacacttttctgcgtgtacacccatggctagaccatcgtacgcatcgttcaacccatctctttcgacaccatcaacaagaacagtttggccaaatttggcacccgcacgggctgctggcatgtagtatggtgcgttagtcatagattcacaaccaccagctacgacaacatcagcattaccacatttgatggattgagcacccaaaatgattgccttcatagcggatgcacagaccttgttaactgtgcttgcaacgatatgattactcaaaccggcagccaaagcaacttgtctggccggagcttggcccaaattggcagaaagaacgttaccaaaaataatttcgtcaaaatccttggatgcatccaattctggaaccttagccaaggcgccttttaaagcaacagcacccaattccactgctgtcttggaggatagagaaccctggaatgaaccaattggggttctggcagtcgatacaatgtaaacgttctgagacatttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactataatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcattatttattagttgtttttaatcttaactgtgtatgaagttttatgtaataaagatagaaagagaaacaaaaaaaaatttttcgtagtatcaattcagctttcgaagacagaatgaaatttaagcagaccatcccgggagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacagtagcaactacaataacaacaacggcggcgtttaaac

SEQ ID NO: 5 - DNA, 2357 bp, synthetic construct
  misc_feature 1..2357: note = ERG8-PGAL-ERG19 insert of pAM497
  source 1..2357: mol_type = other DNA
agattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactataatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgatatgtaacatttttcttttcttttcttttcctttttttacaatagctaatttacgtttccctacggtattggtcggaacgaccaagcttcaatttataaatatcttaattttaacagcagttaccacttgaatgagaaacccgggaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgacctcctcttcagtactaaatctaccaataccaaatctgatggaagaatgggctaatgcatcatccttacccagcgcatgtaaaacataagaaggttctagggaagcagatgtacaggctgaacccgaggataatgcgatatcccttagtgccatcaataaagattctccttccacgtaggcgaaagaaacgttaacacgtttaaac

SEQ ID NO: 6 - DNA, 2121 bp, synthetic construct
  misc_feature 1..2121: note = KanMX-PMET3 region from pAM328
  source 1..2121: mol_type = other DNA
gaattcgcccttntggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctgatcttgaaactgagtaagatgctcagaatacccgtcaagataagagtataatgtagagtaatataccaagtattcagcatattctcctcttcttttgtataaatcacggaagggatgatttataagaaaaatgaatactattacacttcatttaccaccctctgatctagattttccaacgatatgtacgtagtggtataaggtgagggggtccacagatataacatcgtttaatttagtactaacagagacttttgtcacaactacatataagtgtacaaatatagtacagatatgacacacttgtagcgccaacgcgcatcctacggattgctgacagaaaaaaaggtcacgtgaccagaaaagtcacgtgtaattttgtaactcaccgcattctagcggtccctgtcgtgcacactgcactcaacaccataaaccttagcaacctccaaaggaaatcaccgtataacaaagccacagttttacaacttagtctcttatgaagttacttaccaatgagaaatagaggctctttctcgagaaatatgaatatggatatatatatatatatatatatatatatatatatatgtaaacttggttcttttttagcttgtgatctctagcttgggtctctctctgtcgtaacagttgtgatatcgnaagggcgaattc

SEQ ID NO: 7 - DNA, 8425 bp, synthetic construct
  misc_feature 1..8425: note = Plasmid pAM426
  source 1..8425: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtggaagatccgaggcctagctttaacgaacgcagaattttcgagttattaaacttaaaatacgctgaacccgaacatagaaatatcgaatgggaaaaaaaaactgcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatttatttcttttcggataagaaagcaacacctggcaattccttaccttccaataattccaaagaagcaccaccaccagtagagacatgggagacccgggccatggttagatagacatagggtaaactagcaatgatttgatcaaatgcttgtattcatctcccattctcgtaaaattgtctttacctgcatattggacctctaaaaattggcaaagatatataacagccataagtaaaggtcttgggatattctttgttgttaaatactctctgtttatgtctttccaaacgtcctccacttccttataaatcagtgtctgagcatattcttcgttgacattgtattccttcatgtaagattctaaagagcttgaactatgttttctctcctgttccgctttatgagtcatcaggtcatttaatctcctacccagaataccactgtaacggaataaaggcggagcagatacagcccactcaactgattccttagtgaaaatatcgctcattcctagataacaggtagttgttagcaagtttgcaccaccagtgataataactacgggatcgtgctcttcagttgtcggtatgtgtccttcattagcccatttcgcttctaccattagattccttacgaattctttaacgaactccttcccacagttgaataaatcagttctaccttctttggccagaaactcctccatttctgtgtaggtatccatgaataatttgtaaataggcttcatgtattccggcaacgtgtctaagcaggtgatcgaccatctttccacggcttcagtgaaaatctttaactcctcgtaagttccatatgcgtcatacgtgtcatcaataagtgttatcacagcaactgccttagtgaaaaaaactctagctcttgaatactggggttcgtaaccagaacctaaaccccaaaaatagcattcaacgatacgatctctcagacatggggcatttttcttaatatcaaatgccttccaccacttgcatacgtgactcaactcttccttatgtaggctctgcaatagattgaactccagtttagctaactttagcagagttttattatgggagtcttgttgctgatagaagggtatgtactgggcggcctcgatccttggcaatctcttccacaatggttgctttaaagctctctggatttcagtgaataaagcggggtttgtactaaacgcgtcctttgtcataatcgatagccttgatcttgtgaatcccagggcatcttcaagaattatttcgcccggaactctcatggacgtagcctcatataattccaacaatccttcaacatcattcgctaacgattgtttaaaagcaccattcttgtctttatagttattaaacacatcacacgtgacatagtatccttgtttacgcatcagcctaaaccataagctagacctgtcgccattccaattatcaccataggtctcgtaaatacattgcaatgcatgatcaatttcacgttcaaaatgatacggaatacctaaacgttgaatctcgtcaatcagcttcaacaaatttgcatgtttcataggaatatccaatgcttcctttaacaactgtcttacttccttctttagatcgtttactatttgctccacaccctgttcaacttgtttctcataaatcaaaaattgatcgccccaaatagaaggtgggaaatttgcaattggccttataggtttctcttcagtcaaggccattgttttctgcagatccggggttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtactaagatctgctttaatttggccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 8 - DNA, 1763 bp, synthetic construct
  misc_feature 1..1763: note = DNA fragment GAL80-50 to -1-NatR- GAL801309 to 1358
  source 1..1763: mol_type = other DNA
ccagcgtatacaatctcgatagttggtttcccgttctttccactcccgtccacaggaaacagctatgaccatgattacgccaagcttggtaccgagctcggatccactagtaacggccgccagtgtgctggaattcgcccttgtcgacactagtaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgccggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgagtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttctcgaggagctcaagggcaattctgcagatatccatcacactggcggccgctcgagcatgcatctagagggcccaattcgccctatagtgagtcgtattacaattcactggccgtcgttttacaacaagcatcttgccctgtgcttggcccccagtgcagcgaacgttataaaaac

SEQ ID NO: 9 - DNA, 3901 bp, synthetic construct
  misc_feature 1..3901: note = GAL74 to 1021-HPH- GAL11637 to 2587 insert of pAM584
  source 1..3901: mol_type = other DNA
gtttaaacgaggctcacctaacaattcaaaaccaaccaagaatttccgaacagtagctgatctcagtaaaggtgggtagaaatgcatgtgaaaccaactattactcaattcatcaccagtcgcattcaaaggagcctgatggatacccattgagtatgggaaactcgtttcaaataaattatcatacttaatagttagttgctttaaaatcgaggcgaggtcctccttcaccatttggttaaattggctaattgaggcaagcttcttctttgaaatgaccaaggtctcaaatggccagatggcccagtatggaacaacaacaataaaggattcattctccactacgactcttgacttctctcttgattctaatttgacgtaatcggcaaacaaatcagtattgtgttcacgtttatatttatcaaaagatttcaattcttgcgaaacttcactagggatggattctaagcaccaagcttggccatgtggatgtaagttggaacaacccatggctgtacctttgttttcaaatatttggacatatttgaaaggcttatgattttctcttgcttctctggagagatcgtcagtcaatgcttgccaagaattaacaatatgaaccagatctgattgtttcatttgtggaatggttagattatgattggggctaaaacatatgacgaaacaattgcctctcacagattgcactttaagcagcctatttttaagattatcctcattggaatcattctgtggtaaaataggttgatcgagcctaacggcagcataatcattggggaaaatatacgttgattcatatcttgggtttaggttaccagtagctcttttgttaccaggacatagatagcattttggatcatacaatggagctgtgggcttgtaagcagcctcctgttgacctaaccaaggtcttttagctctgtgtggagaaactaagatccatgaatcggttagtggattgtaacgtctatgggaatggctagaaaaatcaaattcttcagcagtgtcgacactagtaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttctcgagactcataactttagcatcacaaaatacgcaataataacgagtagtaacacttttatagttcatacatgcttcaactacttaataaatgattgtatgataatgttttcaatgtaagagatttcgattatccacaaactttaaaacacagggacaaaattcttgatatgctttcaaccgctgcgttttggatacctattcttgacatgatatgactaccattttgttattgtacgtggggcagttgacgtcttatcatatgtcaaagtcatttgcgaagttcttggcaagttgccaactgacgagatgcagtaaaaagagattgccgtcttgaaactttttgtccttttttttttccggggactctacgagaaccctttgtcctactgattaattttgtactgaatttggacaattcagattttagtagacaagcgcgaggaggaaaagaaatgacagaaaaattccgatggacaagaagataggaaaaaaaaaaagctttcaccgatttcctagaccggaaaaaagtcgtatgacatcagaatgaaaaattttcaagttagacaaggacaaaatcaggacaaattgtaaagatataataaactatttgattcagcgccaatttgcccttttccattttccattaaatctctgttctctcttacttatatgatgattaggtatcatctgtataaaactcctttcttaatttcactctaaagcataccccatagagaagatctttcggttcgaagacattcctacgcataataagaataggagggaataatgccagacaatctatcattacatttaagcggctcttcaaaaagattgaactctcgccaacttatggaatcttccaatgagacctttgcgccaaataatgtggatttggaaaaagagtataagtcatctcagagtaatataactaccgaagtttatgaggcatcgagctttgaagtttaaac

SEQ ID NO: 10 - DNA, 66 bp, synthetic construct
  misc_feature 1..66: note = Primer 50-56-pw100-G
  source 1..66: mol_type = other DNA
gagtgaacctgctgcctggcgtgctctgactcagtacatttcatagtggatggcggcgttagtatc

SEQ ID NO: 11 - DNA, 65 bp, synthetic construct
  misc_feature 1..65: note = Primer 50-56-pw101-G
  source 1..65: mol_type = other DNA
cgtgtatacgttttccgcttctgctcttcgtcttttctcttcttccgatatcacaactgttacga

SEQ ID NO: 12 - DNA, 30 bp, synthetic construct
  misc_feature 1..30: note = Primer 61-67-CPK001-G
  source 1..30: mol_type = other DNA
gtttaaactactattagctgaattgccact

SEQ ID NO: 13 - DNA, 46 bp, synthetic construct
  misc_feature 1..46: note = Primer 61-67-CPK002-G
  source 1..46: mol_type = other DNA
actgcaaagtacacatatatcccgggtgtcagctcttttagatcgg

SEQ ID NO: 14 - DNA, 46 bp, synthetic construct
  misc_feature 1..46: note = Primer 61-67-CPK003-G
  source 1..46: mol_type = other DNA
ccgatctaaaagagctgacacccgggatatatgtgtactttgcagt

SEQ ID NO: 15 - DNA, 30 bp, synthetic construct
  misc_feature 1..30: note = Primer 61-67-CPK004-G
  source 1..30: mol_type = other DNA
gtttaaacggcgtcagtccaccagctaaca

SEQ ID NO: 16 - DNA, 30 bp, synthetic construct
  misc_feature 1..30: note = Primer 61-67-CPK005-G
  source 1..30: mol_type = other DNA
gtttaaacttgctaaattcgagtgaaacac

SEQ ID NO: 17 - DNA, 46 bp, synthetic construct
  misc_feature 1..46: note = Primer 61-67-CPK006-G
  source 1..46: mol_type = other DNA
aaagatgaattgaaaagcttcccgggtatggaccctgaaaccacag

SEQ ID NO: 18 - DNA, 46 bp, synthetic construct
  misc_feature 1..46: note = Primer 61-67-CPK007-
  source 1..46: mol_type = other DNA
ctgtggtttcagggtccatacccgggaagcttttcaattcatcttt

SEQ ID NO: 19 - DNA, 30 bp, synthetic construct
  misc_feature 1..30: note = Primer 61-67-CPK008-G
  source 1..30: mol_type = other DNA
gtttaaacccaacaataataatgtcagatc

SEQ ID NO: 20 - DNA, 30 bp, synthetic construct
  misc_feature 1..30: note = Primer 61-67-CPK009-G
  source 1..30: mol_type = other DNA
gtttaaactactcagtatattaagtttcga

SEQ ID NO: 21 - DNA, 70 bp, synthetic construct
  misc_feature 1..70: note = Primer 61-67-CPK010-G
  source 1..70: mol_type = other DNA
atctctcgcaagagtcagactgactcccgggcgtgaataagcttcgggtgacccttatggcattcttttt

SEQ ID NO: 22 - DNA, 70 bp, synthetic construct
  misc_feature 1..70: note = Primer 61-67-CPK011-G
  source 1..70: mol_type = other DNA
aaaaagaatgccataagggtcacccgaagcttattcacgcccgggagtcagtctgactcttgcgagagat

SEQ ID NO: 23 - DNA, 30 bp, synthetic construct
  misc_feature 1..30: note = Primer 61-67-CPK012-G
  source 1..30: mol_type = other DNA
gtttaaacaatttagtgtctgcgatgatga

SEQ ID NO: 24 - DNA, 30 bp, synthetic construct
  misc_feature 1..30: note = Primer 61-67-CPK013-G
  source 1..30: mol_type = other DNA
gtttaaactattgtgagggtcagttatttc

SEQ ID NO: 25 - DNA, 44 bp, synthetic construct
  misc_feature 1..44: note = Primer 61-67-CPK014alt-G
  source 1..44: mol_type = other DNA
gcggggacgaggcaagctaaactttagtatattcttcgaagaaa

SEQ ID NO: 26 - DNA, 44 bp, synthetic construct
  misc_feature 1..44: note = Primer 61-67-CPK015alt-G
  source 1..44: mol_type = other DNA
tttcttcgaagaatatactaaagtttagcttgcctcgtccccgc

SEQ ID NO: 27 - DNA, 60 bp, synthetic construct
  misc_feature 1..60: note = Primer 61-67-CPK016-G
  source 1..60: mol_type = other DNA
caatcaacgtggagggtaattctgctagcctctcccgggtggatggcggcgttagtatcg

SEQ ID NO: 28 - DNA, 60 bp, synthetic construct
  misc_feature 1..60: note = Primer 61-67-CPK017-G
  source 1..60: mol_type = other DNA
cgatactaacgccgccatccacccgggagaggctagcagaattaccctccacgttgattg

SEQ ID NO: 29 - DNA, 30 bp, synthetic construct
  misc_feature 1..30: note = Primer 61-67-CPK018-G
  source 1..30: mol_type = other DNA
gtttaaacgccgccgttgttgttattgtag

SEQ ID NO: 30 - DNA, 30 bp, synthetic construct
  misc_feature 1..30: note = Primer 61-67-CPK019-G
  source 1..30: mol_type = other DNA
gtttaaacttttccaataggtggttagcaa

SEQ ID NO: 31 - DNA, 55 bp, synthetic construct
  misc_feature 1..55: note = Primer 61-67-CPK020-G
  source 1..55: mol_type = other DNA
gggtgacccggcggggacgaggcaagctaaacgtcttcctttctcttaccaaagt

SEQ ID NO: 32 - DNA, 55 bp, synthetic construct
  misc_feature 1..55: note = Primer 61-67-CPK021-G
  source 1..55: mol_type = other DNA
actttggtaagagaaaggaagacgtttagcttgcctcgtccccgccgggtcaccc

SEQ ID NO: 33 - DNA, 62 bp, synthetic construct
  misc_feature 1..62: note = Primer 61-67-CPK022-G
  source 1..62: mol_type = other DNA
aatatcataaaaaaagagaatctttcccgggtggatggcggcgttagtatcgaatcgacagc

SEQ ID NO: 34 - DNA, 62 bp, synthetic construct
  misc_feature 1..62: note = Primer 61-67-CPK023-G
  source 1..62: mol_type = other DNA
gctgtcgattcgatactaacgccgccatccacccgggaaagattctctttttttatgatatt

SEQ ID NO: 35 - DNA, 45 bp, synthetic construct
  misc_feature 1..45: note = Primer 61-67-CPK024-G
  source 1..45: mol_type = other DNA
gtttaaacgtgttaacgtttctttcgcctacgtggaaggagaatc

SEQ ID NO: 36 - DNA, 35 bp, synthetic construct
  misc_feature 1..35: note = Primer 61-67-CPK025-G
  source 1..35: mol_type = other DNA
tccccccgggttaaaaaaaatccttggactagtca

SEQ ID NO: 37 - DNA, 35 bp, synthetic construct
  misc_feature 1..35: note = Primer 61-67-CPK031-G
  source 1..35: mol_type = other DNA
tccccccgggagttatgacaattacaacaacagaa

SEQ ID NO: 38 - DNA, 30 bp, synthetic construct
  misc_feature 1..30: note = Primer 61-67-CPK032-G
  source 1..30: mol_type = other DNA
tccccccgggtatatatatatcattgttat

SEQ ID NO: 39 - DNA, 30 bp, synthetic construct
  misc_feature 1..30: note = Primer 61-67-CPK035-G
  source 1..30: mol_type = other DNA
tccccccgggaaaagtaagtcaaaaggcac

SEQ ID NO: 40 - DNA, 30 bp, synthetic construct
  misc_feature 1..30: note = Primer 61-67-CPK040-G
  source 1..30: mol_type = other DNA
tccccccgggatggtctgcttaaatttcat

SEQ ID NO: 41 - DNA, 45 bp, synthetic construct
  misc_feature 1..45: note = Primer 61-67-CPK041-G
  source 1..45: mol_type = other DNA
tccccccgggtagcttgtacccattaaaagaattttatcatgccg

SEQ ID NO: 42 - DNA, 30 bp, synthetic construct
  misc_feature 1..30: note = Primer 61-67-CPK046-G
  source 1..30: mol_type = other DNA
tccccccgggtttctcattcaagtggtaac

SEQ ID NO: 43 - DNA, 30 bp, synthetic construct
  misc_feature 1..30: note = Primer 61-67-CPK047-G
  source 1..30: mol_type = other DNA
tccccccgggtaaataaagaaaataaagtt

SEQ ID NO: 44 - DNA, 47 bp, synthetic construct
  misc_feature 1..47: note = Primer 61-67-CPK050-G
  source 1..47: mol_type = other DNA
aatttttgaaaattcaatataaatggcttcagaaaaagaaattagga

SEQ ID NO: 45 - DNA, 47 bp, synthetic construct
  misc_feature 1..47: note = Primer 61-67-CPK051-G
  source 1..47: mol_type = other DNA
tcctaatttctttttctgaagccatttatattgaattttcaaaaatt

SEQ ID NO: 46 - DNA, 51 bp, synthetic construct
  misc_feature 1..51: note = Primer 61-67-CPK052-G
  source 1..51: mol_type = other DNA
agttttcaccaattggtctgcagccattatagttttttctccttgacgtta

SEQ ID NO: 47 - DNA, 51 bp, synthetic construct
  misc_feature 1..51: note = Primer 61-67-CPK053-G
  source 1..51: mol_type = other DNA
taacgtcaaggagaaaaaactataatggctgcagaccaattggtgaaaact

SEQ ID NO: 48 - DNA, 47 bp, synthetic construct
  misc_feature 1..47: note = Primer 61-67-CPK054-G
  source 1..47: mol_type = other DNA
aatttttgaaaattcaatataaatgaaactctcaactaaactttgtt

SEQ ID NO: 49 - DNA, 47 bp, synthetic construct
  misc_feature 1..47: note = Primer 61-67-CPK055-G
  source 1..47: mol_type = other DNA
aacaaagtttagttgagagtttcatttatattgaattttcaaaaatt

SEQ ID NO: 50 - DNA, 47 bp, synthetic construct
  misc_feature 1..47: note = Primer 61-67-CPK056-G
  source 1..47: mol_type = other DNA
aatttttgaaaattcaatataaatgtctcagaacgtttacattgtat

SEQ ID NO: 51 - DNA, 47 bp, synthetic construct
  misc_feature 1..47: note = Primer 61-67-CPK057-G
  source 1..47: mol_type = other DNA
atacaatgtaaacgttctgagacatttatattgaattttcaaaaatt

SEQ ID NO: 52 - DNA, 51 bp, synthetic construct
  misc_feature 1..51: note = Primer 61-67-CPK058-G
  source 1..51: mol_type = other DNA
tgcagaagttaagaacggtaatgacattatagttttttctccttgacgtta

SEQ ID NO: 53 - DNA, 51 bp, synthetic construct
  misc_feature 1..51: note = Primer 61-67-CPK059-G
  source 1..51: mol_type = other DNA
taacgtcaaggagaaaaaactataatgtcattaccgttcttaacttctgca

SEQ ID NO: 54 - DNA, 47 bp, synthetic construct
  misc_feature 1..47: note = Primer 61-67-CPK060-G
  source 1..47: mol_type = other DNA
aatttttgaaaattcaatataaatgtcagagttgagagccttcagtg

SEQ ID NO: 55 - DNA, 47 bp, synthetic construct
  misc_feature 1..47: note = Primer 61-67-CPK061-G
  source 1..47: mol_type = other DNA
cactgaaggctctcaactctgacatttatattgaattttcaaaaatt

SEQ ID NO: 56 - DNA, 51 bp, synthetic construct
  misc_feature 1..51: note = Primer 61-67-CPK062-G
  source 1..51: mol_type = other DNA
ggtaacggatgctgtgtaaacggtcattatagttttttctccttgacgtta

SEQ ID NO: 57 - DNA, 51 bp, synthetic construct
  misc_feature 1..51: note = Primer 61-67-CPK063-G
  source 1..51: mol_type = other DNA
taacgtcaaggagaaaaaactataatgaccgtttacacagcatccgttacc

SEQ ID NO: 58 - DNA, 47 bp, synthetic construct
  misc_feature 1..47: note = Primer 61-67-CPK064-G
  source 1..47: mol_type = other DNA
aatttttgaaaattcaatataaatgactgccgacaacaatagtatgc

SEQ ID NO: 59 - DNA, 47 bp, synthetic construct
  misc_feature 1..47: note = Primer 61-67-CPK065-G
  source 1..47: mol_type = other DNA
gcatactattgttgtcggcagtcatttatattgaattttcaaaaatt

SEQ ID NO: 60 - DNA, 70 bp, synthetic construct
  misc_feature 1..70: note = Primer 61-67-CPK066-G
  source 1..70: mol_type = other DNA
ggtaagacggttgggttttatcttttgcagttggtactattaagaacaatcacaggaaacagctatgacc

SEQ ID NO: 61 - DNA, 70 bp, synthetic construct
  misc_feature 1..70: note = Primer 61-67-CPK067-G
  source 1..70: mol_type = other DNA
ttgcgttttgtactttggttcgctcaattttgcaggtagataatcgaaaagttgtaaaacgacggccagt

SEQ ID NO: 62 - DNA, 44 bp, synthetic construct
  misc_feature 1..44: note = Primer 91-014-CPK231-G
  source 1..44: mol_type = other DNA
ttgtgatgctaaagttatgagtctcgagaagttaagattatatg

SEQ ID NO: 63 - DNA, 44 bp, synthetic construct
  misc_feature 1..44: note = Primer 91-014-CPK232-G
  source 1..44: mol_type = other DNA
catataatcttaacttctcgagactcataactttagcatcacaa

SEQ ID NO: 64 - DNA, 28 bp, synthetic construct
  misc_feature 1..28: note = Primer 91-014-CPK233-G
  source 1..28: mol_type = other DNA
gtttaaacttcaaagctcgatgcctcat

SEQ ID NO: 65 - DNA, 28 bp, synthetic construct
  misc_feature 1..28: note = Primer 91-014-CPK236-G
  source 1..28: mol_type = other DNA
gtttaaacgaggctcacctaacaattca

SEQ ID NO: 66 - DNA, 44 bp, synthetic construct
  misc_feature 1..44: note = Primer 91-014-CPK237-G
  source 1..44: mol_type = other DNA
gatgtgtattactagtgtcgacactgctgaagaatttgattttt

SEQ ID NO: 67 - DNA, 44 bp, synthetic construct
  misc_feature 1..44: note = Primer 91-014-CPK238-G
  source 1..44: mol_type = other DNA
aaaaatcaaattcttcagcagtgtcgacactagtaatacacatc

SEQ ID NO: 68 - DNA, 1761 bp, synthetic construct
  misc_feature 1..1761: note = DNA fragment GAL11 to 48-NatR- GAL11500 to 1550
  source 1..1761: mol_type = other DNA
atgactaaatctcattcagaagaagtgattgtacctgagttcaattctcacaggaaacagctatgaccatgattacgccaagcttggtaccgagctcggatccactagtaacggccgccagtgtgctggaattcgcccttgtcgacactagtaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgccggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgagtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttctcgaggagctcaagggcaattctgcagatatccatcacactggcggccgctcgagcatgcatctagagggcccaattcgccctatagtgagtcgtattacaattcactggccgtcgttttacaacaagtaccctaagatcactgatgctgagctagaaaatgctatcatcgtctc

SEQ ID NO: 69 - DNA, 7348 bp, synthetic construct
  misc_feature 1..7348: note = Vector pAM178
  source 1..7348: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtgcggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 70 - DNA, 4883 bp, synthetic construct
  misc_feature 1..4883: note = T7-FS-FRT-Kan-FRT cassette
  source 1..4883: mol_type = other DNA
gctgggcgatctgttgcgcgaagtggtatggccgacagatgtgtccacgctggatgttgatgcaatggtggtacgcgaaaccactcaccgtttcagccgcttatcctttcaccgggcaatggtcgggcgacgtttgccgcttctgaaaaccgcctcgggcctgacctggctggccttttgcccggaacaagaccgcaaggaattaatcgaaatgttagcctcccgccccggtgatgactatcaactggcacgggaaccgttaaagctggaagccattctggcgcgcgcgcgcaaagagggttacggacagaactaccgcggctgggatcaggaggagaagatcgcctctatcgccgtaccgctgcgcagtgaacaacgggtgattggctgtctgaatctggtgtatatggcgagcgcaatgaccattgaacaggcagcggaaaagcatcttccggcgctacaacgggtagcaaaacagatcgaagaaggggttgaatcgcaggctattctggtggccggaaggcgaagcggcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatggacactctgccgatctcttccgtaagcttttcttcctctacttccccgctggtagtcgatgacaaggtttctaccaaacctgatgtaattcgtcacactatgaacttcaacgcatctatctggggcgatcagttcctgacttatgatgaaccggaagatctggtaatgaaaaagcaactggtagaagaactgaaagaagaagttaaaaaggaactgatcaccattaagggtagcaacgaaccgatgcagcacgtgaaactgattgaactgatcgatgcggttcagcgtctgggtattgcttatcattttgaagaggaaatcgaggaagctctgcaacacatccacgtaacctacggcgaacaatgggtggataaagagaatctgcagtctatcagcctgtggttccgcctgctgcgtcagcaaggtttcaatgtctcttctggcgttttcaaagacttcatggatgaaaagggcaaattcaaggaatccctgtgtaacgatgcgcaaggtatcctggcactgtacgaagcggccttcatgcgtgtggaagacgaaaccattctggacaacgcgctggaattcactaaagtgcatctggacatcatcgcgaaagatccgtcctgcgactcctctctgcgtactcagatccatcaagcgctgaaacagccgctgcgtcgtcgcctggcacgtattgaggctctgcactatatgccgatttaccagcaggaaacctctcacgacgaagtcctgctgaaactggctaaactggacttcagcgttctgcaatctatgcacaagaaagaactgtcccacatctgcaaatggtggaaagatctggatctgcaaaacaaactgccgtacgttcgtgaccgtgttgttgagggctatttttggattctgtccatctactatgaaccacagcacgcgcgtactcgcatgtttctgatgaaaacctgcatgtggctggttgtcctggacgacacctttgacaactatggtacgtacgaagaactggaaatcttcacccaggccgtggaacgttggtctatttcctgcctggatatgctgccggaatacatgaaactgatctatcaagaactggttaacctgcacgtggaaatggaagagtctctggagaaagaaggtaaaacttaccagatccactacgtcaaggagatggcgaaagaactggtccgtaactatctggtcgaggcgcgttggctgaaagagggctatatgccgactctggaagaatacatgagcgtatccatggttaccggcacctacggcctgatgattgcgcgttcctacgtcggccgtggtgatattgttaccgaagatacctttaagtgggtttcttcctacccgccgatcatcaaagcgtcttgtgtcatcgttcgcctgatggacgacatcgtttctcacaaagaggagcaagaacgtggtcacgtagcatctagcatcgaatgctactccaaagaatccggcgcgtccgaagaagaagcttgcgaatacatcagccgtaaagttgaagatgcctggaaagttatcaaccgcgaaagcctgcgtccgacggcggtcccgtttccgctgctgatgccggcaatcaacctggcacgcatgtgtgaggttctgtacagcgtgaacgatggttttactcacgcggaaggtgacatgaagagctatatgaagagcttcttcgtacaccctatggtcgtatgagagctcggtacccaaactctatgactgagttaattaagcagttcatacaggcgcgccgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttctagagctgcctcgcgcgtttcggtgatgacggtcctctgaccggttggacgttaaaaaatatccccggcaactgacacgctaccggggatttttttatcattctgagcttgtctgtaagcggatgccgggagcagacaagcccgtgaagttcctattctctagaaagtataggaacttcagagcgctttgacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactagttaaggggatcttgaagttcctattccgaagttcctattctctagaaagtataggaacttcacatgcagctcccggagacggtcacgcgatcgcattataaaaattgcctgatacgctgcgcttatcaggcctacaagttcagcgatctacattagccgcatccggcatgaacaaagcgcaggaacaagcgtcgcatcatgcctctttgacccacagctgcggaaaacgtactggtgcaaaacgcagggttatgatcatcagcccaacgacgcacagcgcatgaaatgcccagtccatcaggtaattgccgctgatactacgcagcacgccagaaaaccacggggcaagcccggcgatgataaaaccgattccctgcataaacgccaccagcttgccagcaatagccggttgcacagagtgatcgagcgccagcagcaaacagagcggaaacgcgccgcccagacctaacccacacaccatcgcccacaataccggcaattgcatcggcagccagataaagccgcagaaccccaccagttgtaacaccagcgccagcattaacagtttgcgccgatcctgatggcgagccatagcaggcatcagcaaagctcctgcggcttgcccaagcgtcatcaatgccagt

SEQ ID NO: 71 - DNA, 1332 bp, synthetic construct
  misc_feature 1..1332: note = Mevalonate kinase codon-optimized for expression in Escherichia coli
  source 1..1332: mol_type = other DNA
atgtctctgccattcctgacgtctgcgccaggtaaggtgatcatcttcggcgagcactctgcggtgtacaataagccggccgtcgccgcctctgtgtctgcgttacgcacctacctgctgatcagcgaatcttctgcaccggacacgatcgagctggactttccggacatcagcttcaaccacaagtggagcatcaacgacttcaacgcgatcacggaggaccaggtgaacagccaaaagctggccaaagcccagcaagcaaccgacggtctgtctcaggagctggtgtctctgctggacccgctgttagcgcagttaagcgagagcttccattaccacgccgcgttctgcttcctgtacatgttcgtttgcctgtgcccgcacgcaaagaacatcaagttcagcctgaagagcacgctgccgattggcgcaggcttaggctctagcgcatctatcagcgtgagcctggcgctggcgatggcctatctgggtggcctgattggcagcaacgacctggagaaactgagcgaaaacgacaagcacatcgtgaaccagtgggcctttatcggcgagaagtgcattcatggcaccccgagcggcattgacaacgcagttgccacgtatggcaacgccctgctgttcgagaaagacagccacaacggcacgatcaacacgaacaacttcaagttcctggacgacttcccggcgatcccgatgattctgacctacacccgtatcccacgcagcaccaaggatttagtcgcccgcgtgcgtgttttagtcaccgaaaagttcccggaggtgatgaagccgatcctggacgcgatgggcgagtgcgcgctgcagggtctggagatcatgaccaagctgagcaagtgcaagggcaccgacgatgaggcggtggagaccaacaatgagctgtacgagcagctgctggagctgatccgtatcaatcacggcctgctggtctctatcggtgtgtctcacccgggcctggaactgatcaaaaacctgagcgacgacctgcgcattggctctacgaaattaacgggtgcaggtggcggtggctgctctttaacgctgctgcgccgtgacattacgcaggagcaaatcgacagcttcaagaagaagctgcaggacgacttcagctacgagacgttcgagacggacctgggcggcacgggctgttgcctgctgagcgccaaaaatctgaacaaggacctgaagatcaaaagcctggtgttccagctgttcgaaaacaagacgaccacgaagcagcagatcgacgacctgttactgccgggtaacaccaatctgccgtggacgtcttaa

SEQ ID NO: 72 - DNA, 5220 bp, synthetic construct
  misc_feature 1..5220: note = Plasmid pAM618
  source 1..5220: mol_type = other DNA
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctgcggccgcgagctttcctaaaaaaggttatccaccttttttaggatgcaagagctatggaaggtctctataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacccatatggaattcgagctcggtacccggggatcctctagagtcgacctgcaggcatgcaagcttgagctcggtacccaaactctatgactgagttaattaagcagttcatacaggcgcgccgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttctagagctgcctcgcgcgtttcggtgatgacggtcctctgaccggttggacgttaaaaaatatccccggcaactgacacgctaccggggatttttttatcattctgagcttgtctgtaagcggatgccgggagcagacaagcccgtgaagttcctattctctagaaagtataggaacttcagagcgctttgacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactagttaaggggatcttgaagttcctattccgaagttcctattctctagaaagtataggaacttcacatgcagctcccggagacggtcacgcgatcgcttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtgagacgccacgtaccagcggccgcgtcgacaagcttatgcatccatgcagttggccatgcctacatcgagtaccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 73 - DNA, 1356 bp, synthetic construct
  misc_feature 1..1356: note = Phosphomevalonate kinase codon-optimized for expression in Escherichia coli
  source 1..1356: mol_type = other DNA
atgagcgaattacgtgcattcagcgcgccaggtaaggcactgctggccggtggctacctggtgttagacaccaagtacgaggcgttcgtcgtcggcttatctgcccgtatgcatgcagttgcccacccgtatggtagcctgcagggctctgacaagttcgaagtgcgtgtgaagagcaagcagttcaaggacggcgagtggctgtaccacattagcccaaagagcggcttcatcccggttagcattggtggcagcaagaacccatttatcgagaaggtcattgccaacgtcttcagctacttcaagccgaatatggacgattactgcaaccgcaacctgttcgtcatcgacattttcagcgacgacgcgtaccacagccaagaggactctgttacggagcatcgtggtaaccgccgcctgagcttccacagccatcgcattgaggaggtgccgaagacgggtctgggttctagcgccggtttagttaccgtcttaacgacggcgttagcgagcttcttcgtgagcgacctggagaacaacgtggacaagtaccgcgaagtgattcataacctggcgcaggtggcacattgtcaggcccaaggtaagattggctctggttttgatgtggcagcggccgcctatggctctatccgctatcgccgctttccgccggccctgatcagcaatctgccggacatcggctctgcgacgtatggtagcaaactggcgcatctggtggacgaggaggactggaacatcaccattaagtctaatcacctgccgagcggcttaacgttatggatgggcgatatcaagaacggcagcgaaacggttaagctggtgcagaaagtgaaaaactggtacgacagccacatgccggaaagcctgaagatttacacggagctggaccacgccaatagccgtttcatggatggtctgagcaagctggaccgcctgcacgaaacccacgacgactacagcgaccagatcttcgagagcctggagcgcaatgactgcacctgccagaagtacccggagatcacggaggtccgcgatgccgtggcaacgattcgccgtagcttccgcaaaattacgaaggagagcggcgcggatatcgaaccaccggtccagacgtctctgctggacgactgtcaaaccttaaagggcgtgttaacgtgcctgattccgggcgcgggtggttacgacgccattgccgtcatcacgaaacaggacgtcgatctgcgcgcacaaacggccaacgacaaacgtttcagcaaagtccaatggctggatgttacgcaggccgactggggtgttcgcaaggagaaggacccggaaacgtatctggataagtga

(19) United States
(12) Patent Application Publication
(10) Pub. No.: US 2025/0122551 A1
(43) Pub. Date: Apr. 17, 2025

(54) PRODUCTION OF ISOPRENOIDS

(71) Applicant: AMYRIS, INC., Emeryville, CA (US)

(72) Inventors: Hiroko TSURUTA, Oakland, CA (US); Jacob R. LENIHAN, Emeryville, CA (US); Rika REGENTIN, Hayward, CA (US)

(21) Appl. No.: 18/915,097

(22) Filed: Oct. 14, 2024

Related U.S. Application Data
(63) Continuation of application No. 18/208,030, filed on Jun. 9, 2023, now Pat. No. 12,146,176.
Application No. 18/208,030 is a continuation of application No. 12/234,589, filed on Sep. 19, 2008, now Pat. No. 11,725,225.
(60) Provisional application No. 60/994,790, filed on Sep. 20, 2007.
(60) Provisional application No. 61/049,350, filed on Apr. 30, 2008.

(51) Int. Cl. C12P 23/00; C12N 1/32; C12P 5/00

(52) U.S. Cl. CPC C12P 23/00; C12N 1/32; C12P 5/007; Y02E 50/10

(57) ABSTRACT

Provided herein are methods for a robust production of isoprenoids via one or more biosynthetic pathways. Also provided herein are nucleic acids, enzymes, expression vectors, and genetically modified host cells for carrying out the subject methods. Also provided herein are fermentation methods for high productivity of isoprenoids from genetically modified host cells.

### CROSS-REFERENCE TO RELATED APPLICATIONS

This application is a continuation of U.S. application Ser. No. 18/208,030, filed Jun. 9, 2023, which is a continuation application of U.S. application Ser. No. 12/234,589, filed Sep. 19, 2008 (now issued as U.S. Pat. No. 11,725,225), which claims the benefit of U.S. Provisional Application Nos. 60/994,790, filed Sep. 20, 2007, and 61/049,350, filed Apr. 30, 2008, all of which are incorporated herein by reference in their entirety.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 29, 2023, is named “107345.00907.xml” and is 157,295 bytes in size. The sequence listing contained in this .XML file is part of the specification and is hereby incorporated by reference herein in its entirety.

### FIELD OF THE INVENTION

Provided herein are, among others, compositions and methods for a robust production of isoprenoids. Also provided herein are nucleic acids, enzymes, expression vectors, and genetically modified host cells for carrying out the methods. Also provided herein are fermentation methods for high productivity of isoprenoids from genetically modified host cells.

### BACKGROUND OF THE INVENTION

Isoprenoids are ubiquitous in nature. They comprise a diverse family of over 40,000 individual products, many of which are vital to living organisms. Isoprenoids serve to maintain cellular fluidity, electron transport, and other metabolic functions. A vast number of natural and synthetic isoprenoids are useful as pharmaceuticals, cosmetics, perfumes, pigments and colorants, fungicides, antiseptics, nutraceuticals, and fine chemical intermediates.

An isoprenoid product is typically composed of repeating five carbon isopentenyl diphosphate (IPP) units, although irregular isoprenoids and polyterpenes have been reported. In nature, isoprenoids are synthesized by consecutive condensations of their precursor IPP and its isomer dimethylallyl pyrophosphate (DMAPP). Two pathways for these precursors are known. Eukaryotes, with the exception of plants, generally use the mevalonate-dependent (MEV) pathway to convert acetyl coenzyme A (acetyl-CoA) to IPP, which is subsequently isomerized to DMAPP. Prokaryotes, with some exceptions, typically employ only the mevalonate-independent or deoxyxylulose-5-phosphate (DXP) pathway to produce IPP and DMAPP. Plants use both the MEV pathway and the DXP pathway. See Rohmer et al. (1993) Biochem. J. 295:517-524; Lange et al. (2000) Proc. Natl. Acad. Sci. USA 97 (24):13172-13177; Rohdich et al. (2002) Proc. Natl. Acad. Sci. USA 99:1158-1163.

Traditionally, isoprenoids have been manufactured by extraction from natural sources such as plants, microbes, and animals. However, the yield by way of extraction is usually very low due to a number of profound limitations. First, most isoprenoids accumulate in nature in only small amounts. Second, the source organisms in general are not amenable to the large-scale cultivation that is necessary to produce commercially viable quantities of a desired isoprenoid. Third, the requirement of certain toxic solvents for isoprenoid extraction necessitates special handling and disposal procedures, and thus complicating the commercial production of isoprenoids.

The elucidation of the MEV and DXP metabolic pathways has made biosynthetic production of isoprenoids feasible. For instance, microbes have been engineered to overexpress a part of or the entire mevalonate pathway for production of an isoprenoid named amorpha-4,11-diene (U.S. Pat. Nos. 7,172,886 and 7,192,751). Other efforts have focused on balancing the pool of glyceraldehyde-3-phosphate and pyruvate, or on increasing the expression of 1-deoxy-D-xylulose-5-phosphate synthase (dxs) and IPP isomerase (idi). See Farmer et al. (2001) Biotechnol. Prog. 17:57-61: Kajiwara et al. (1997) Biochem. J. 324:421-426; and Kim et al. (2001) Biotechnol. Bioeng. 72:408-415.

Nevertheless, given the very large quantities of isoprenoid products needed for many commercial applications, there remains a need for expression systems and fermentation procedures that produce even more isoprenoids than available with current technologies. Optimal redirection of microbial metabolism toward isoprenoid production requires that the introduced biosynthetic pathway is properly engineered both to funnel carbon to isoprenoid production efficiently and to prevent build up of toxic levels of metabolic intermediates over a sustained period of time. Provided herein are compositions and methods that address this need and provide related advantages as well.

### SUMMARY OF THE INVENTION

Provided herein are compositions and methods for a robust production of isoprenoids. Non-limiting examples of suitable isoprenoids include: hemiterpenes (derived from 1 isoprene unit) such as isoprene; monoterpenes (derived from 2 isoprene units) such as myrcene; sesquiterpenes (derived from 3 isoprene units) such as amorpha-4,11-diene; diterpenes (derived from four isoprene units) such as taxadiene: triterpenes (derived from 6 isoprene units) such as squalene; tetraterpenes (derived from 8 isoprenoids) such as β-carotene; and polyterpenes (derived from more than 8 isoprene units) such as polyisoprene.

In one aspect, a method for producing an isoprenoid compound is provided wherein the method comprises:
(a) obtaining a plurality of host cells that are capable of making the isoprenoid compound comprising a chromosomally integrated heterologous nucleic acid sequence encoding an enzyme of the MEV or DXP pathway;
(b) culturing the host cells in a medium under conditions wherein the host cells use ethanol as a carbon source and make the isoprenoid compound; and
(c) recovering the isoprenoid compound from the medium.

In some embodiments, the ethanol that is consumed by the host cells as the carbon source is made by the host cell. In other embodiments, the ethanol that is consumed by the host cells as the carbon source is exogenously supplied to the medium.

In another aspect, a method for making an isoprenoid compound is provided which comprises:
(a) obtaining a plurality of host cells that are capable of making the isoprenoid compound;
(b) culturing the host cells in a medium comprising ethanol in an amount equal to or greater than about 1 gram per liter of medium for at least four hours; and
(c) recovering the isoprenoid compound from the medium.

In yet another aspect, a method for making an isoprenoid compound is provided which comprises:
(a) obtaining a plurality of yeast cells that are capable of making the isoprenoid compound;
(b) culturing the yeast cells to build biomass by providing a bolus of a carbon source to the medium;
(c) maintaining the cells under conditions whereby the yeast cells have an ethanol consumption rate equal to or greater than about 0.01 gram per ethanol per gram of dry cell weight per hour for at least four hours; and
(d) recovering the isoprenoid compound from the medium.

In some embodiments, the host cells make the isoprenoid compound using the MEV pathway. In other embodiments, the host cells make the isoprenoid compound using the DXP pathway.

In other embodiments, the host cells are cultured or maintained for at least some period of time under oxygen limited conditions. In still other embodiments, the host cells are cultured or maintained for at least some period of time under phosphate limited conditions.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 is a schematic representation of the mevalonate (“MEV”) pathway for the production of isopentenyl pyrophosphate (“IPP”).

FIG. 2 is a schematic representation of the 1-deoxy-D-xylulose 5-diphosphate (“DXP”) pathway for the production of isopentenyl pyrophosphate (“IPP”) and dimethylallyl pyrophosphate (“DMAPP”). Dxs is 1-deoxy-D-xylulose-5-phosphate synthase; Dxr is 1-deoxy-D-xylulose-5-phosphate reductoisomerase (also known as IspC); IspD is 4-diphosphocytidyl-2C-methyl-D-erythritol synthase; IspE is 4-diphosphocytidyl-2C-methyl-D-erythritol synthase; IspF is 2C-methyl-D-erythritol 2,4-cyclodiphosphate synthase; IspG is 1-hydroxy-2-methyl-2-(E)-butenyl 4-diphosphate synthase (IspG); and ispH is isopentenyl/dimethylallyl diphosphate synthase.

FIG. 3 is a schematic representation of the conversion of isopentenyl pyrophosphate (“IPP”) and dimethylallyl pyrophosphate (“DMAPP”) to geranyl pyrophosphate (“GPP”), farnesyl pyrophosphate (“FPP”), and geranylgeranyl pyrophosphate (“GGPP”), and the synthesis of various isoprenoids.

FIGS. 4A-4G show maps of DNA fragments: ERG20-PGAL-tHMGR (FIG. 4A), ERG13-PGAL-tHMGR (FIG. 4B), IDI1-PGAL-tHMGR (FIG. 4C), ERG10-PGAL-ERG12 (FIG. 4D), ERG8-PGAL-ERG19 (FIG. 4E), GAL7^{4 to 1021}-HPH-GAL1^{1637 to 2587} (not shown), GAL80-50 to-1-NatR-GAL801309 to 1358 (FIG. 4F), and GAL1 (1 to 48)-NatR-GAL1 (1500 to 1550) (FIG. 4G).

FIG. 5 shows a map of plasmid pAM404.

FIG. 6 shows cell growth and amorpha-4,11-diene (AD) production by strain Y337 under carbon restriction using either a glucose feed or a glucose/ethanol mixed feed.

FIG. 7A shows a diagram of a CO2 control feed algorithm. FIG. 7B shows carbon dioxide evolution rate, substrate delivery, growth, and production of amorpha-4,11-diene by strain Y293 using an ethanol pulse feed.

FIG. 8 shows cell growth and amorpha-4,11-diene production by strain Y293 under carbon restriction using a concentrated glucose feed for initial growth followed by an ethanol feed for production.

FIGS. 9A through 9E show ethanol production/consumption, feed rate, growth, carbon evolution and oxygen utilization rates, and farnesene production by strain Y677 in fed batch, carbon-restricted fermentation with an ethanol only feed. in the presence or absence of methyl oleate.

FIGS. 10A through 10D show dissolved oxygen concentration, growth, ethanol production/consumption, and amorpha-4,11-diene production by strain Y283 at different degrees of oxygen limitation.

FIGS. 10E through 10G show growth, ethanol production/consumption, and farnesene production by strain Y352 at different degrees of oxygen limitation.

FIG. 11 shows per cell amorpha-4,11-diene productivity by strain Y337 in shake flasks under carbon restriction with varying concentrations of KH2PO4.

FIG. 12 shows a fed-batch fermentor feed (A), and cell growth (B) and amorpha-4,11-diene production (C) by strain Y337 under carbon-and phosphate-restriction using a glucose feed.

FIG. 13 shows cell growth (A) and amorpha-4,11-diene production (B) by strain Y337 under carbon- and phosphate-restriction using a glucose/ethanol mixed feed.

FIG. 14 illustrates the generation of 100 nucleotide long genomic locus-specific sequences flanking promoter-gene-FRT-Kan-FRT cassettes useful in the integration of heterologous nucleotide sequences into the genome of Escherichia coli.

FIGS. 15 shows a map of plasmid pAM618.

### DETAILED DESCRIPTION OF THE INVENTION

### Definitions

Unless defined otherwise, all technical and scientific terms used herein have the same meaning as commonly understood by one of ordinary skill in the art. Reference is made here to a number of terms that shall be defined to have the following meanings:

The term “optional” or “optionally” means that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where the event or circumstance does not occur.

The terms “metabolic pathway” is used herein to refer to a catabolic pathway or an anabolic pathway. Anabolic pathways involve constructing a larger molecule from smaller molecules, a process requiring energy. Catabolic pathways involve breaking down of larger molecules, often releasing energy.

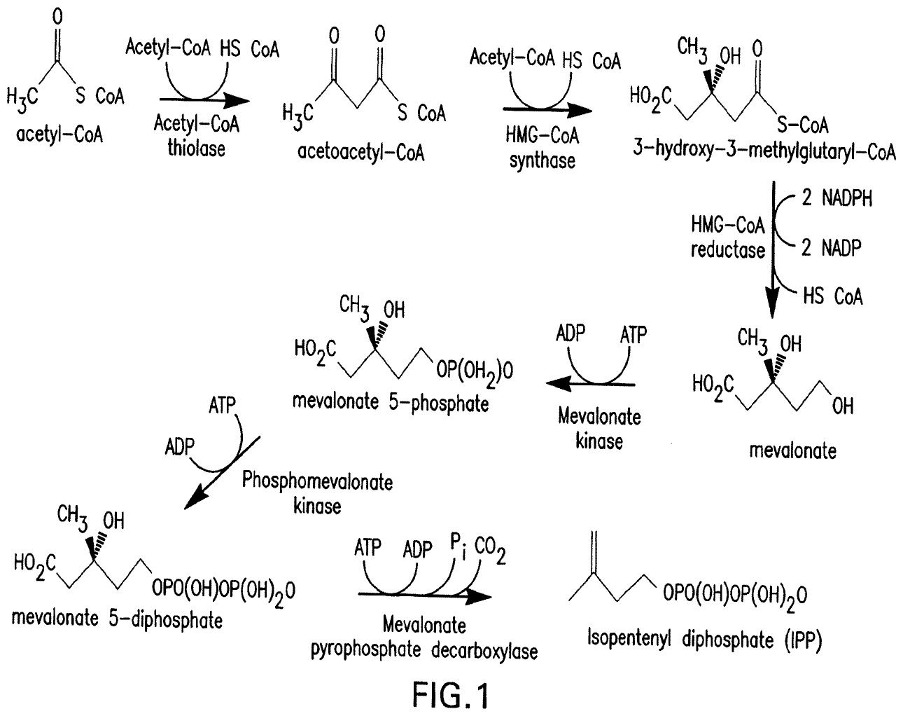

The term “mevalonate pathway” or “MEV pathway” is used herein to refer to the biosynthetic pathway that converts acetyl-CoA to IPP. The MEV pathway is illustrated schematically in FIG. 1.

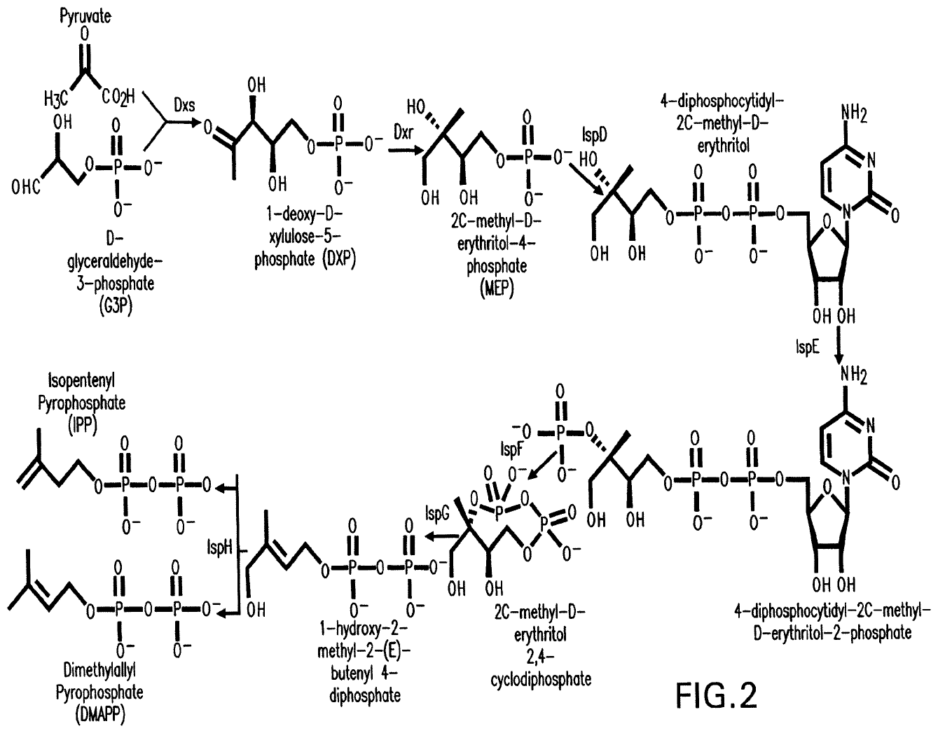

The term “deoxyxylulose 5-phosphate pathway” or “DXP pathway” is used herein to refer to the pathway that converts glyceraldehyde-3-phosphate and pyruvate to IPP and DMAPP. The DXP pathway is illustrated schematically in FIG. 2.

The word “pyrophosphate” is used interchangeably herein with “diphosphate”.

The terms “expression vector” or “vector” refer to a nucleic acid that transduces, transforms, or infects a host cell, thereby causing the cell to produce nucleic acids and/or proteins other than those that are native to the cell, or to express nucleic acids and/or proteins in a manner that is not native to the cell.

The term “endogenous” refers to a substance or process that occurs naturally, e.g., in a non-recombinant host cell.

The terms “enzymatic pathway for making isopentenyl pyrophosphate” refers to any pathway capapble of producing isopentyl pyrophosphate, including, without limitation, either the mevalonate pathway or the DXP pathway.

The term “nucleic acid” refers to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically, or biochemically modified, non-natural, or derivatized nucleotide bases.

The term “operon” is used to refer to two or more contiguous nucleotide sequences that each encode a gene product such as a RNA or a protein, and the expression of which are coordinately regulated by one or more controlling elements (for example, a promoter).

The term “gene product” refers to RNA encoded by DNA (or vice versa) or protein that is encoded by an RNA or DNA, where a gene will typically comprise one or more nucleotide sequences that encode a protein, and may also include introns and other non-coding nucleotide sequences.

The term “protein” refers to a polymeric form of amino acids of any length, which can include coded and non-coded amino acids, chemically or biochemically modified or derivatized amino acids, and polypeptides having modified peptide backbones.

The term “heterologous nucleic acid” as used herein refers to a nucleic acid wherein at least one of the following is true: (a) the nucleic acid is foreign (“exogenous”) to (that is, not naturally found in) a given host cell; (b) the nucleic acid comprises a nucleotide sequence that is naturally found in (that is, is “endogenous to”) a given host cell, but the nucleotide sequence is produced in an unnatural (for example, greater than expected or greater than naturally found) amount in the cell; (c) the nucleic acid comprises a nucleotide sequence that differs in sequence from an endogenous nucleotide sequence, but the nucleotide sequence encodes the same protein (having the same or substantially the same amino acid sequence) and is produced in an unnatural (for example, greater than expected or greater than naturally found) amount in the cell; or (d) the nucleic acid comprises two or more nucleotide sequences that are not found in the same relationship to each other in nature (for example, the nucleic acid is recombinant).

A “transgene” refers to a gene that is exogenously introduced into a host cell. It can comprise an endogenous or exogenous, or heterologous nucleic acid.

The term “recombinant host” (also referred to as a “genetically modified host cell” or “genetically modified host microorganism”) denotes a host cell that comprises a heterologous nucleic acid provided herein.

The term “exogenous nucleic acid” refers to a nucleic acid that is exogenously introduced into a host cell, and hence is not normally or naturally found in and/or produced by a given cell in nature.

The term “regulatory element” refers to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” refers to a permanent or transient genetic change induced in a cell following introduction of new nucleic acid. Genetic change (“modification”) can be accomplished either by incorporation of the new DNA into the genome of the host cell, or by transient or stable maintenance of the new DNA as an episomal element. In eukaryotic cells, a permanent genetic change is generally achieved by introduction of the DNA into the genome of the cell. In prokaryotic cells, a permanent genetic change can be introduced into the chromosome or via extrachromosomal elements such as plasmids and expression vectors, which may contain one or more selectable markers to aid in their maintenance in the recombinant host cell.

The term “operably linked” refers to a juxtaposition wherein the components so described are in a relationship permitting them to function in their intended manner. For instance, a promoter is operably linked to a nucleotide sequence if the promoter affects the transcription or expression of the nucleotide sequence.

The term “host cell” and “host microorganism” are used interchangeably herein to refer to any archae, bacterial, or eukaryotic living cell into which a heterologous nucleic acid can be or has been inserted. The term also relates to the progeny of the original cell, which may not necessarily be completely identical in morphology or in genomic or total DNA complement to the original parent, due to natural, accidental, or deliberate mutation.

The term “synthetic” as used in reference to nucleic acids means the annealing of chemically synthesized oligonucleotide building blocks to form gene segments, which are then enzymatically assembled to construct the entire gene. Synthesis of nucleic acids via “chemical means” means that the component nucleotides were assembled in vitro.

The term “natural” as applied to a nucleic acid, a cell, or an organism, refers to a nucleic acid, cell, or organism that is found in nature. For example, a polypeptide or polynucleotide sequence that is present in a non-pathological (un-diseased) organism that can be isolated from a source in nature and that has not been intentionally modified by a human in the laboratory is natural.

The term “naturally occurring” as applied to a nucleic acid, an enzyme, a cell, or an organism, refers to a nucleic acid, enzyme, cell, or organism that is found in nature. For example, a polypeptide or polynucleotide sequence that is present in an organism that can be isolated from a source in nature and that has not been intentionally modified by a human in the laboratory is naturally occurring.

The term “biologically active fragment” as applied to a protein, polypeptide or enzyme refers to

functional portion(s) of the proteins or polypeptide or enzyme. Functionally equivalents may have variant amino acid sequences may arise, e.g., as a consequence of codon redundancy and functional equivalency which are known to occur naturally within nucleic acid sequences and the proteins thus encoded. Functionally equivalent proteins or peptides may alternatively be constructed via the application of recombinant DNA technology, in which changes in the protein structure may be engineered, based on considerations of the properties of the amino acids being exchanged.

The terms “isoprenoid”, “isprenoid compound”, “isoprenoid product”, “terpene”, “terpene compound”, “terpenoid”, and “terpenoid compound” are used interchangeably herein. They refer to compounds that are capable of being derived from IPP.

The singular forms “a,” “and,” and “the” include plural referents unless the context clearly dictates otherwise. Thus, for example, reference to “an expression vector” includes a single expression vector as well as a plurality of expression vectors, and reference to “the host cell” includes reference to one or more host cells, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless otherwise indicated, the embodiments provided herein are not limited to particular sequences, expression vectors, enzymes, host microorganisms, or processes, as such may vary in accordance with the understanding of those of ordinary skill in the art in view of the teaching herein. Terminology used herein is for purposes of describing particular embodiments only and is not intended to be limiting.

IPP Pathways

The host cells provided herein comprise or utilize the MEV pathway, the DXP pathway or both to synthesize IPP and its isomer, DMAPP. Provided herein is the host cell includes at least one chromosomally integrated heterologous nucleic acid sequence encoding an enzyme of the MEV or DXP pathways. In other embodiments, the host cell includes at least one heterologous nucleic acid sequence encoding a plurality of enzymes of the MEV or DXP pathways. In still other embodiments, the host cell includes a plurality of heterologous nucleic acid sequences encoding all of the MEV pathway enzymes. In yet other embodiments, the host cell includes a plurality of heterologous nucleic acid sequences that encodes all of the DXP pathway enzymes.

In general, eukaryotes other than plants use the MEV isoprenoid pathway exclusively to convert acetyl-CoA to IPP, which is subsequently isomerized to DMAPP. Prokaryotes, with some exceptions, use the mevalonate-independent or DXP pathway to produce IPP and DMAPP separately through a branch point. Plants use both the MEV and DXP pathways for IPP synthesis.

MEV Pathway

A schematic representation of the MEV pathway is described in FIG. 1. In general, the pathway comprises six steps.

In the first step, two molecules of acetyl-coenzyme A are enzymatically combined to form acetoacetyl-CoA. An enzyme known to catalyze this step is, for example, acetyl-CoA thiolase (also known as acetyl-CoA acetyltransferase). Illustrative examples of nucleotide sequences include but are not limited to the following GenBank accession numbers and the organism from which the sequences derived: (NC_000913 REGION: 2324131 . . . 2325315; Escherichia coli), (D49362; Paracoccus denitrificans), and (L20428; Saccharomyces cerevisiae).

In the second step of the MEV pathway, acetoacetyl-CoA is enzymatically condensed with another molecule of acetyl-CoA to form 3-hydroxy-3-methylglutaryl-CoA (HMG-COA). An enzyme known to catalyze this step is, for example, HMG-CoA synthase. Illustrative examples of nucleotide sequences include but are not limited to: (NC_001145. complement 19061 . . . 20536; Saccharomyces cerevisiae), (X96617; Saccharomyces cerevisiae), (X83882; Arabidopsis thaliana), (AB037907; Kitasatospora griseola), (BT007302; Homo sapiens), and (NC_002758, Locus tag SAV2546, GeneID 1122571; Staphylococcus aureus).

In the third step, HMG-CoA is enzymatically converted to mevalonate. An enzyme known to catalyze this step is, for example, HMG-CoA reductase. Illustrative examples of nucleotide sequences include but are not limited to: (NM_206548; Drosophila melanogaster), (NC_002758, Locus tag SAV2545, GeneID 1122570; Staphylococcus aureus), (NM_204485; Gallus gallus), (AB015627; Streptomyces sp. KO 3988), (AF542543; Nicotiana attenuata), (AB037907; Kitasatospora griseola), (AX128213, providing the sequence encoding a truncated HMGR; Saccharomyces cerevisiae), and (NC_001145; complement (115734 . . . 118898; Saccharomyces cerevisiae).

In the fourth step, mevalonate is enzymatically phosphorylated to form mevalonate 5-phosphate. An enzyme known to catalyze this step is, for example, mevalonate kinase. Illustrative examples of nucleotide sequences include but are not limited to: (L77688; Arabidopsis thaliana), and (X55875; Saccharomyces cerevisiae).

In the fifth step, a second phosphate group is enzymatically added to mevalonate 5-phosphate to form mevalonate 5-pyrophosphate. An enzyme known to catalyze this step is, for example, phosphomevalonate kinase. Illustrative examples of nucleotide sequences include but are not limited to: (AF429385; Hevea brasiliensis), (NM_006556; Homo sapiens), and (NC_001145. complement 712315 . . . 713670; Saccharomyces cerevisiae).

In the sixth step, mevalonate 5-pyrophosphate is enzymatically converted into IPP. An enzyme known to catalyze this step is, for example, mevalonate pyrophosphate decarboxylase. Illustrative examples of nucleotide sequences include but are not limited to: (X97557; Saccharomyces cerevisiae), (AF290095; Enterococcus faecium), and (U49260; Homo sapiens).

If IPP is to be converted to DMAPP, then a seventh step is required. An enzyme known to catalyze this step is, for example, IPP isomerase. Illustrative examples of nucleotide sequences include but are not limited to: (NC_000913, 3031087 . . . 3031635; Escherichia coli), and (AF082326; Haematococcus pluvialis). If the conversion to DMAPP is required, an increased expression of IPP isomerase ensures that the conversion of IPP into DMAPP does not represent a rate-limiting step in the overall pathway.

### DXP Pathway

A schematic representation of the DXP pathway is described in FIG. 2. In general, the DXP pathway comprises seven steps. In the first step, pyruvate is condensed with D-glyceraldehyde 3-phosphate to make 1-deoxy-D-xylulose-5-phosphate. An enzyme known to catalyze this step is, for example, 1-deoxy-D-xylulose-5-phosphate synthase. Illustrative examples of nucleotide sequences include but are not limited to: (AF035440; Escherichia coli), (NC_002947, locus tag PP0527; Pseudomonas putida KT2440), (CP000026, locus tag SPA2301; Salmonella enterica Paratyphi, see ATCC 9150), (NC_007493, locus tag RSP_0254; Rhodobacter sphaeroides 2.4.1), (NC_005296, locus tag RPA0952; Rhodopseudomonas palustris CGA009), (NC_004556, locus tag PD1293; Xylella fastidiosa Temecula1), and (NC_003076, locus tag AT5G11380; Arabidopsis thaliana).

In the second step, 1-deoxy-D-xylulose-5-phosphate is converted to 2C-methyl-D-erythritol-4-phosphate. An enzyme known to catalyze this step is, for example, 1-deoxy-D-xylulose-5-phosphate reductoisomerase. Illustrative examples of nucleotide sequences include but are not limited to: (AB013300; Escherichia coli), (AF148852; Arabidopsis thaliana), (NC_002947, locus tag U.S. Plant Pat. No. 1,597; Pseudomonas putida KT2440), (AL939124, locus tag SCO5694; Streptomyces coelicolor A3(2)), (NC_007493, locus tag RSP_2709; Rhodobacter sphaeroides 2.4.1), and (NC_007492, locus tag Pf1_1107; Pseudomonas fluorescens PfO-1).

In the third step, 2C-methyl-D-erythritol-4-phosphate is converted to 4-diphosphocytidyl-2C-methyl-D-erythritol. An enzyme known to catalyze this step is, for example, 4-diphosphocytidyl-2C-methyl-D-erythritol synthase. Illustrative examples of nucleotide sequences include but are not limited to: (AF230736; Escherichia coli), (NC_007493, locus_tag RSP_2835; Rhodobacter sphaeroides 2.4.1), (NC_003071, locus_tag AT2G02500; Arabidopsis thaliana), and (NC_002947, locus_tag PP1614; Pseudomonas putida KT2440).

In the fourth step, 4-diphosphocytidyl-2C-methyl-D-erythritol is converted to 4-diphosphocytidyl-2C-methyl-D-erythritol-2-phosphate. An enzyme known to catalyze this step is, for example, 4-diphosphocytidyl-2C-methyl-D-erythritol kinase. Illustrative examples of nucleotide sequences include but are not limited to: (AF216300; Escherichia coli) and (NC_007493, locus_tag RSP_1779; Rhodobacter sphaeroides 2.4.1).

In the fifth step, 4-diphosphocytidyl-2C-methyl-D-erythritol-2-phosphate is converted to 2C-methyl-D-erythritol 2, 4-cyclodiphosphate. An enzyme known to catalyze this step is, for example, 2C-methyl-D-erythritol 2, 4-cyclodiphosphate synthase. Illustrative examples of nucleotide sequences include but are not limited to: (AF230738; Escherichia coli), (NC_007493, locus_tag RSP_6071; Rhodobacter sphaeroides 2.4.1), and (NC_002947, locus_tag PP1618; Pseudomonas putida KT2440).

In the sixth step, 2C-methyl-D-erythritol 2, 4-cyclodiphosphate is converted to 1-hydroxy-2-methyl-2-(E)-butenyl-4-diphosphate. An enzyme known to catalyze this step is, for example, 1-hydroxy-2-methyl-2-(E)-butenyl-4-diphosphate synthase. Illustrative examples of nucleotide sequences include but are not limited to: (AY033515; Escherichia coli), (NC_002947, locus_tag PP853; Pseudomonas putida KT2440), and (NC_007493, locus_tag RSP_2982; Rhodobacter sphaeroides 2.4.1).

In the seventh step, 1-hydroxy-2-methyl-2-(E)-butenyl-4-diphosphate is converted into either IPP or its isomer, DMAPP. An enzyme known to catalyze this step is, for example, isopentyl/dimethylallyl diphosphate synthase. Illustrative examples of nucleotide sequences include but are not limited to: (AY062212; Escherichia coli) and (NC_002947, locus_tag PP606; Pseudomonas putida KT2440).

In some embodiments, “cross talk” (or interference) between the host cell's own metabolic processes and those processes involved with the production of IPP as provided herein are minimized or eliminated entirely. For example, cross talk is minimized or eliminated entirely when the host microorganism relies exclusively on the DXP pathway for synthesizing IPP, and a MEV pathway is introduced to provide additional IPP. Such a host organisms would not be equipped to alter the expression of the MEV pathway enzymes or process the intermediates associated with the MEV pathway. Organisms that rely exclusively or predominately on the DXP pathway include, for example, Escherichia coli.

In some embodiments, the host cell produces IPP via the MEV pathway, either exclusively or in combination with the DXP pathway. In other embodiments, a host's DXP pathway is functionally disabled so that the host cell produces IPP exclusively through a heterologously introduced MEV pathway. The DXP pathway can be functionally disabled by disabling gene expression or inactivating the function of one or more of the DXP pathway enzymes.

### Host Cells

Illustrative examples of suitable host cells for use provided herein include any archae, prokaryotic, or eukaryotic cell. Examples of an archae cell include, but are not limited to those belonging to the genera: Aeropyrum, Archaeglobus, Halobacterium, Methanococcus, Methanobacterium, Pyrococcus, Sulfolobus, and Thermoplasma. Illustrative examples of archae strains include but are not limited to: Aeropyrum pernix, Archaeoglobus fulgidus, Methanococcus jannaschii, Methanobacterium thermoautotrophicum, Pyrococcus abyssi, Pyrococcus horikoshii, Thermoplasma acidophilum, Thermoplasma volcanium.

Examples of a procaryotic cell include, but are not limited to those belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas.

Illustrative examples of prokaryotic bacterial strains include but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus, and the like.

In general, if a bacterial host cell is used, a non-pathogenic strain is preferred. Illustrative examples of non-pathogenic strains include but are not limited to: Bacillus subtilis, Escherichia coli, Lactibacillus acidophilus, Lactobacillus helveticus, Pseudomonas aeruginosa, Pseudomonas mevalonii, Pseudomonas pudita, Rhodobacter sphaeroides, Rodobacter capsulatus, Rhodospirillum rubrum, and the like.

Examples of eukaryotic cells include but are not limited to fungal cells. Examples of fungal cell include, but are not limited to those belonging to the genera: Aspergillus, Candida, Chrysosporium, Cryotococcus, Fusarium, Kluyveromyces, Neotyphodium, Neurospora, Penicillium, Pichia, Saccharomyces, Trichoderma and Xanthophyllomyces (formerly Phaffia).

Illustrative examples of eukaryotic strains include but are not limited to: Aspergillus nidulans,

Aspergillus niger, Aspergillus oryzae, Candida albicans, Chrysosporium lucknowense, Fusarium graminearum, Fusarium venenatum, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccaromyces bayanus, Saccaromyces boulardi, Saccharomyces cerevisiae, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, Trichoderma reesei and Xanthophyllomyces dendrorhous (formerly Phaffia rhodozyma).

In general, if a eukaryotic cell is used, a non-pathogenic strain is preferred. Illustrative examples of non-pathogenic strains include but are not limited to: Fusarium graminearum, Fusarium venenatum, Pichia pastoris, Saccaromyces boulardi, and Saccaromyces cerevisiae.

In addition, certain strains have been designated by the Food and Drug Administration as GRAS or Generally Regarded As Safe. These strains include: Bacillus subtilis, Lactibacillus acidophilus, Lactobacillus helveticus, and Saccharomyces cerevisiae.

### Isoprenoid Compounds

The host cells provided herein are used to make isoprenoids. Specific isprenoid compounds are made from IPP or DMAPP and may require additional finishing enzymes. Non-limiting examples of suitable isoprenoids include: hemiterpenes (derived from 1 isoprene unit) such as isoprene; monoterpenes (derived from 2 isoprene units) such as myrcene; sesquiterpenes (derived from 3 isoprene units) such as amorpha-4,11-diene; diterpenes (derived from four isoprene units) such as taxadiene; triterpenes (derived from 6 isoprene units) such as squalene; tetraterpenes (derived from 8 isoprenoids) such as β-carotene; and polyterpenes (derived from more than 8 isoprene units) such as polyisoprene. In some embodiments, the isoprenoid is not a carotenoid. In other embodiments, the isoprenoid is a C₅-C₂₀ isoprenoid. Illustrative examples of specific C₅-C₂₀ isoprenoid compounds and their respective finishing enzymes are further described below.

### C₅ Compounds

C₅ compounds provided herein generally are derived from IPP or DMAPP. These compounds are also known as hemiterpenes because they are derived from a single isoprene unit (IPP or DMAPP).

### Isoprene

Isoprene, whose structure is

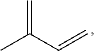

is found in many plants. Isoprene is made from IPP by isoprene synthase. Illustrative examples of suitable nucleotide sequences include but are not limited to: (AB198190; Populus alba) and (AJ294819; Polulus alba x Polulus tremula).

### C₁₀ Compounds

C₁₀ compounds provided herein generally derived from geranyl pyrophosphate (GPP) which is made by the condensation of IPP with DMAPP. An enzyme known to catalyze this step is, for example, geranyl pyrophosphate synthase. These C₁₀ compounds are also known as monoterpenes because they are derived from two isoprene units.

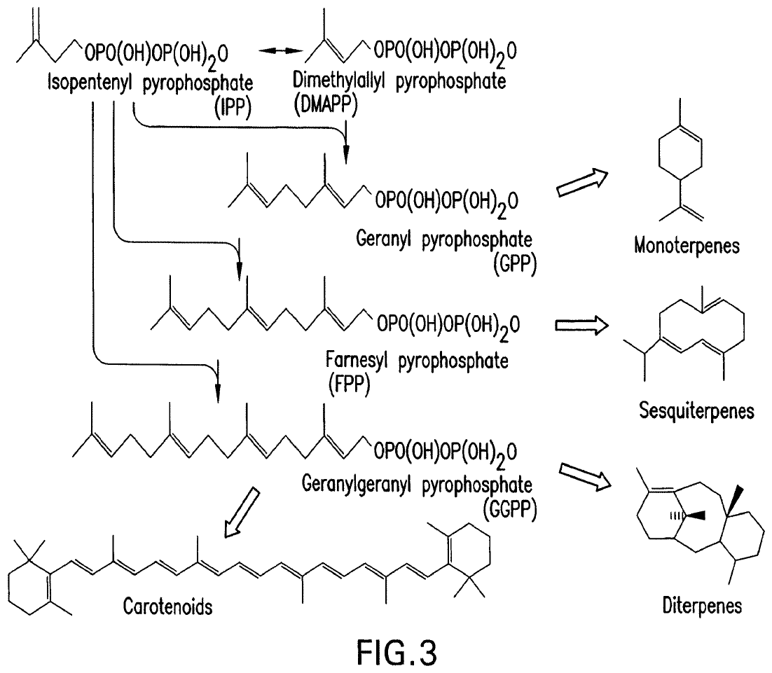

FIG. 3 shows schematically how IPP and DMAPP can produce GPP, which can be further processed to a monoterpene.

Illustrative examples of nucleotide sequences for geranyl pyrophosphate synthase include but are not limited to: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP11092; Bacillus cereus; ATCC 14579), (AJ243739; Citrus sinensis), (AY534745; Clarkia breweri), (AY953508; Ips pini), (DQ286930; Lycopersicon esculentum), (AF182828; Mentha x piperita), (AF182827; Mentha x piperita), (MPI249453; Mentha x piperita), (PZE431697, Locus CAD24425; Paracoccus zeaxanthinifaciens), (AY866498; Picrorhiza kurrooa), (AY351862; Vitis vinifera), and (AF203881, Locus AAF12843; Zymomonas mobilis).

GPP is then subsequently converted to a variety of C₁₀ compounds. Illustrative examples of C₁₀ compounds include but are not limited:

### Carene

Carene, whose structure is

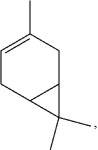

is found in the resin of many trees, particularly pine trees. Carene is made from GPP from carene synthase. Illustrative examples of suitable nucleotide sequences include but are not limited to: (AF461460, REGION 43 . . . 1926; Picea abies) and (AF527416, REGION: 78 . . . 1871; Salvia stenophylla).

### Geraniol

Geraniol (also known as rhodnol), whose structure is

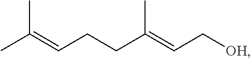

is the main component of oil-of-rose and palmarosa oil. It also occurs in geranium, lemon, and citronella. Geraniol is made from GPP by geraniol synthase. Illustrative examples of suitable nucleotide sequences include but are not limited to: (AJ457070; Cinnamomum tenuipilum), (AY362553; Ocimum basilicum), (DQ234300; Perilla frutescens strain 1864), (DQ234299; Perilla citriodora strain 1861), (DQ234298; Perilla citriodora strain 4935), and (DQ088667; Perilla citriodora)

### Linalool

Linalool, whose structure is

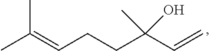

is found in many flowers and spice plants such as coriander seeds. Linalool is made from GPP by linalool synthase. Illustrative examples of a suitable nucleotide sequence include but are not limited to: (AF497485; Arabidopsis thaliana), (AC002294, Locus AAB71482; Arabidopsis thaliana), (AY059757; Arabidopsis thaliana), (NM_104793; Arabidopsis thaliana), (AF154124; Artemisia annua), (AF067603; Clarkia breweri), (AF067602; Clarkia concinna), (AF067601; Clarkia breweri), (U58314; Clarkia breweri), (AY840091; Lycopersicon esculentum), (DQ263741; Lavandula angustifolia), (AY083653; Mentha citrate), (AY693647; Ocimum basilicum), (XM_463918; Oryza sativa), (AP004078, Locus BAD07605; Oryza sativa), (XM_463918, Locus XP_463918; Oryza sativa), (AY917193; Perilla citriodora), (AF271259; Perilla frutescens), (AY473623; Picea abies), (DQ195274; Picea sitchensis), and (AF444798; Perilla frutescens var. crispa cultivar No. 79).

### Limonene

Limonene, whose structure is

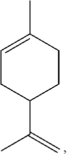

is found in the rind of citrus fruits and peppermint. Limonene is made from GPP by limonene synthase. Illustrative examples of suitable nucleotide sequences include but are not limited to: (+)-limonene synthases (AF514287, REGION: 47 . . . 1867; Citrus limon) and (AY055214, REGION: 48 . . . 1889; Agastache rugosa) and (−)-limonene synthases (DQ195275, REGION: 1 . . . 1905; Picea sitchensis), (AF006193, REGION: 73 . . . 1986; Abies grandis), and (MHC4SLSP, REGION: 29 . . . 1828; Mentha spicata).

### Myrcene

Myrcene, whose structure is

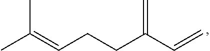

is found in the essential oil in many plants including bay, verbena, and myrcia from which it gets its name. Myrcene is made from GPP by myrcene synthase. Illustrative examples of suitable nucleotide sequences include but are not limited to: (U87908; Abies grandis), (AY195609; Antirrhinum majus), (AY195608; Antirrhinum majus), (NM_127982; Arabidopsis thaliana TPS10), (NM_113485; Arabidopsis thaliana ATTPS-CIN), (NM_113483; Arabidopsis thaliana ATTPS-CIN), (AF271259; Perilla frutescens), (AY473626; Picea abies), (AF369919; Picea abies), and (AJ304839; Quercus ilex).

### Ocimene

α- and β-Ocimene, whose structures are

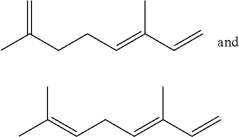

respectively, are found in a variety of plants and fruits including Ocimum basilicum and is made from GPP by ocimene synthase. Illustrative examples of suitable nucleotide sequences include but are not limited to: (AY195607; Antirrhinum majus), (AY195609; Antirrhinum majus), (AY195608; Antirrhinum majus), (AK221024; Arabidopsis thaliana), (NM_113485; Arabidopsis thaliana ATTPS-CIN), (NM_113483; Arabidopsis thaliana ATTPS-CIN), (NM_117775; Arabidopsis thaliana ATTPS03), (NM_001036574; Arabidopsis thaliana ATTPS03), (NM_127982; Arabidopsis thaliana TPS10), (AB110642; Citrus unshiu CitMTSL4), and (AY575970; Lotus corniculatus var. japonicus).

### α-Pinene

α-Pinene, whose structure is

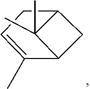

is found in pine trees and eucalyptus. α-Pinene is made from GPP by α-pinene synthase. Illustrative examples of suitable nucleotide sequences include but are not limited to: (+) α-pinene synthase (AF543530, REGION: 1 . . . 1887; Pinus taeda), (−) α-pinene synthase (AF543527, REGION: 32 . . . 1921; Pinus taeda), and (+)/(−)α-pinene synthase (AGU87909, REGION: 6111892; Abies grandis).

### β-Pinene

β-Pinene, whose structure is

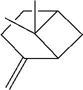

is found in pine trees, rosemary, parsley, dill, basil, and rose. β-Pinene is made from GPP by β-pinene synthase. Illustrative examples of suitable nucleotide sequences include but are not limited to: (−) β-pinene synthases (AF276072, REGION: 1 . . . 1749; Artemisia annua) and (AF514288, REGION: 26 . . . 1834; Citrus limon).

### Sabinene

Sabinene, whose structure is

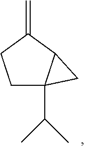

is found in black pepper, carrot seed, sage, and tea trees. Sabinene is made from GPP by sabinene synthase. An illustrative example of a suitable nucleotide sequence includes but is not limited to AF051901, REGION: 26 . . . 1798 from Salvia officinalis.

### γ-Terpinene

γ-Terpinene, whose structure is

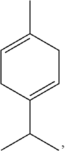

is a constituent of the essential oil from citrus fruits. Biochemically, γ-terpinene is made from GPP by a γ-terpinene synthase. Illustrative examples of suitable nucleotide sequences include: (AF514286, REGION: 30 . . . 1832 from Citrus limon) and (AB110640, REGION 1 . . . 1803 from Citrus unshiu).

### Terpinolene

Terpinolene, whose structure is

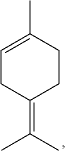

is found in black currant, cypress, guava, lychee, papaya, pine, and tea. Terpinolene is made from GPP by terpinolene synthase. An illustrative example of a suitable nucleotide sequence includes but is not limited to AY906866, REGION: 10 . . . 1887 from Pseudotsuga menziesii.

### C₁₅ Compounds

C₁₅ compounds provided herein generally derive from farnesyl pyrophosphate (FPP) which is made by the condensation of two molecules of IPP with one molecule of DMAPP. An enzyme known to catalyze this step is, for example, farnesyl pyrophosphate synthase. These C₁₅ compounds are also known as sesquiterpenes because they are derived from three isoprene units.

FIG. 3 shows schematically how IPP and DMAPP can be combined to produce FPP, which can be further processed to a sesquiterpene.

Illustrative examples of nucleotide sequences for farnesyl pyrophosphate synthase include but are not limited to: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella fujikuroi), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_008022, Locus YP_598856; Streptococcus pyogenes MGAS10270), (NC_008023, Locus YP_600845; Streptococcus pyogenes MGAS2096), (NC_008024, Locus YP_602832; Streptococcus pyogenes MGAS10750), and (MZEFPS; Zea mays).

Alternatively, FPP can also be made by adding IPP to GPP. Illustrative examples of nucleotide sequences encoding for an enzyme capable of this reaction include but are not limited to: (AE000657, Locus AAC06913; Aquifex aeolicus VF5), (NM_202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC_002940, Locus NP_873754; Haemophilus ducreyi 35000HP), (L42023, Locus AAC23087; Haemophilus influenzae Rd KW20), (J05262; Homo sapiens), (YP_395294; Lactobacillus sakei subsp. sakei 23K), (NC_005823, Locus YP_000273; Leptospira interrogans serovar Copenhageni str. Fiocruz L1-130), (AB003187; Micrococcus luteus), (NC_002946, Locus YP_208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_004556, Locus NP 779706; Xylella fastidiosa Temecula1).

FPP is then subsequently converted to a variety of C₁₅ compounds. Illustrative examples of C₁₅ compounds include but are not limited to:

### Amorphadiene

Amorphadiene, whose structure is

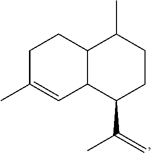

is a precursor to artemisinin which is made by Artemisia anna. Amorphadiene is made from FPP by amorphadiene synthase. An illustrative example of a suitable nucleotide sequence is SEQ ID NO. 37 of U.S. Pat. No. 7,192,751.

### α-Farnesene

α-Farnesene, whose structure is

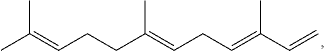

is found in various biological sources including but not limited to the Dufour's gland in ants and in the coating of apple and pear peels. α-Farnesene is made from FPP by α-farnesene synthase. Illustrative examples of suitable nucleotide sequences include but are not limited to DQ309034 from Pyrus communis cultivar d'Anjou (pear; gene name AFS1) and AY182241 from Malus domestica (apple; gene AFS1). Pechouus et al., Planta 219 (1):84-94 (2004).

### β-Farnesene

β-Farnesene, whose structure is

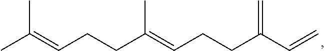

is found in various biological sources including but not limited to aphids and essential oils such as from peppermint. In some plants such as wild potato, β-farnesene is synthesized as a natural insect repellent. β-Farnesene is made from FPP by β-farnesene synthase. Illustrative examples of suitable nucleotide sequences include but is not limited to GenBank accession number AF024615 from Mentha x piperita (peppermint; gene Tspa11), and AY835398 from Artemisia annua. Picaud et al., Phytochemistry 66 (9): 961-967 (2005).

### Farnesol

Farnesol, whose structure is

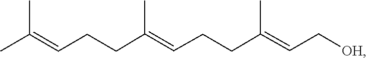 [SMILES: CC(C)=CCC/C(C)=C/CC/C(C)=C/CO]

is found in various biological sources including insects and essential oils such as from cintronella, neroli, cyclamen, lemon grass, tuberose, and rose. Farnesol is made from FPP by a hydroxylase such as farnesol synthase. Illustrative examples of suitable nucleotide sequences include but are not limited to GenBank accession number AF529266 from Zea mays and YDR481C from Saccharomyces cerevisiae (gene Pho8). Song, L., Applied Biochemistry and Biotechnology 128:149-158 (2006).

### Nerolidol

Nerolidol, whose structure is

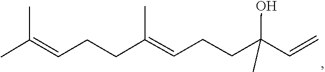 [SMILES: C=CC(C)(O)CC/C=C(\C)CCC=C(C)C]

is also known as peruviol, and is found in various biological sources including as essential oils such as from neroli, ginger, jasmine, lavender, tea tree, and lemon grass. Nerolidol is made from FPP by a hydroxylase such as nerolidol synthase. An illustrative example of a suitable nucleotide sequence includes but is not limited to AF529266 from Zea mays (maize; gene tps1).

### Patchoulol

Patchoulol, whose structure is

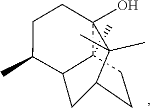

is also known as patchouli alcohol and is a constituent of the essential oil of Pogostemon patchouli. Patchouliol is made from FPP by patchouliol synthase. An illustrative example of a suitable nucleotide sequence includes but is not limited to AY508730 REGION: 1 . . . 1659 from Pogostemon cablin.

### Valencene

Valencene, whose structure is

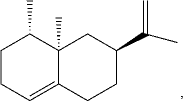

is one of the main chemical components of the smell and flavour of oranges and is found in orange peels. Valencene is made from FPP by nootkatone synthase. Illustrative examples of a suitable nucleotide sequence includes but is not limited to AF441124 REGION: 1 . . . 1647 from Citrus sinensis and AY917195 REGION: 1 . . . 1653 from Perilla frutescens.

### C₂₀ Compounds

C₂₀ compounds provided herein generally derived from geranylgeraniol pyrophosphate (GGPP) which is made by the condensation of three molecules of IPP with one molecule of DMAPP. An enzyme known to catalyze this step is, for example, geranylgeranyl pyrophosphate synthase. These C₂₀ compounds are also known as diterpenes because they are derived from four isoprene units.

FIG. 3 shows schematically how IPP and DMAPP can be combined to produce GGPP, which can be further processed to a diterpene, or can be further processed to produce a carotenoid.

Illustrative examples of nucleotide sequences for geranylgeranyl pyrophosphate synthase include but are not limited to: (ATHGERPYRS; Arabidopsis thaliana), (BT005328; Arabidopsis thaliana), (NM_119845; Arabidopsis thaliana), (NZ_AAJM01000380, Locus ZP_00743052; Bacillus thuringiensis serovar israelensis, ATCC 35646 sq1563), (CRGGPPS; Catharanthus roseus), (NZ_AABF02000074, Locus ZP_00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB017971; Homo sapiens), (MCI276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_007759, Locus YP_461832; Syntrophus aciditrophicus SB), and (NC_006840, Locus YP_204095; Vibrio fischeri ES114).

Alternatively, GGPP can also be made by adding IPP to FPP. Illustrative examples of nucleotide sequences encoding an enzyme capable of this reaction include but are not limited to: (NM_112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_004350, Locus NP_721015; Streptococcus mutans UA159).

GGPP is then subsequently converted to a variety of C₂₀ isoprenoids. Illustrative examples of C₂₀ compounds include but are not limited to:

### Geranylgeraniol

Geranylgeraniol, whose structure is

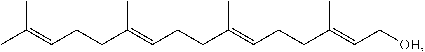 [SMILES: CC(C)=CCC/C(C)=C/CC/C(C)=C/CC/C(C)=C/CO]

is a constituent of wood oil from Cedrela toona and of linseed oil. Geranylgeraniol can be made by e.g., adding to the expression constructs a phosphatase gene after the gene for a GGPP synthase.

### Abietadiene

Abietadiene encompasses the following isomers:

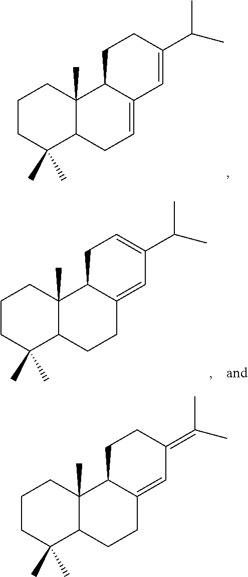

and is found in trees such as Abies grandis. Abietadiene is made by abietadiene synthase. An illustrative example of a suitable nucleotide sequence includes but are not limited to: (U50768; Abies grandis) and (AY473621; Picea abies).

### C2₀₊ Compounds

C₂₀₊ compounds are also within the scope provided herein. Illustrative examples of such compounds include sesterterpenes (C₂₅ compound made from five isoprene units), triterpenes (C₃₀ compounds made from six isoprene units), and tetraterpenes (C₄₀ compound made from eight isoprene units). These compounds are made by using similar methods described herein and substituting or adding nucleotide sequences for the appropriate synthase(s).

### High Yields of Isoprenoid Compounds

Provided herein are compositions and methods for a robust production of isoprenoids by culturing or maintaining the host cells under conditions in which ethanol is used as a carbon source. Using the methods described herein, the host cells produce more than about 5 grams of isoprenoid per liter of fermentation reaction mixture (5 g/L). In other embodiments, more than about 10 g/L, more than about 15 g/L, more than about 20 g/L, more than 25 g/L is produced, or more than about 30 g/L of isoprenoid is produced.

Alternatively isoprenoid production can be expressed in terms of specific productivity instead of yields. For example, using the methods described herein, the host cells produce more about 50 milligrams of isoprenoid per gram of dry host cells. In other embodiments, more than about 100 milligrams per gram dry cell weight, more than about 150 milligrams per gram dry cell weight, more than about 200 milligrams per gram dry cell weight, more than about 250 milligrams per gram dry cell weight, more than about 500 milligrams per gram dry cell weight, more than about 750 milligrams per gram dry cell weight, or more than about 1000 milligrams per gram dry cell weight of isoprenoid is produced.

Whether the production level is expressed in terms of yield or specific productivity, production occurs in less than about 120 hours, less than about 96 hours, less than about 72 hours, preferably less than about 48 hours, or even less than about 24 hours.

The methods provided herein can be carried out in a batch, a fed-batch, or a continuous process. A batch process is typically a closed process where all of the raw materials are added at the beginning of the process. A fed-batch process is typically a closed process where the carbon source and/or other substrates are added in increments throughout the process. A fed-batch process allows for greater control of the medium and the growth of the microorganisms. A continuous process can be considered an open system where medium is continuously added and product is simultaneously removed.

Processes in between fed-batch and continuous processes can also be used. For example, in one embodiment, the process is begun as a fed-batch process, and an organic layer, is placed in contact with the culturing medium while the process continues. Isoprenoids, which typically have a higher solubility in an organic solution than in an aqueous solution, are extracted out of the medium into the organic layer. Because product is removed from the medium, this method has characteristics of both a fed-batch and a continuous process.

Product removal through an organic overlay (e.g. dodecane, isopropyl myristate, methyl oleate and the like) can often lead to increases in isoprenoid production. Product removal can lead to production increases and is desirable particulary where product accumulation leads to pathway inhibition. In certain embodiments, the organic layer can be formed by the isoprenoid product itself. This occurs where the isoprenoid is produced in excess of its saturation point and form a layer separable from the aqueous medium.

In some embodiments, ethanol is the sole carbon source for host cells. In other embodiments, the carbon source includes both ethanol and a non-ethanol carbon source. In still other embodiments, the non-ethanol carbon source is a carbohydrate.

Illustrative examples of carbohydrates include monosaccharides, disaccharides, and combinations thereof. Some non-limiting examples of suitable monosaccharides include glucose, galactose, mannose, fructose, ribose, and combinations thereof. Some non-limiting examples of suitable disaccharides include sucrose, lactose, maltose, trehalose, cellobiose, and combinations thereof. Some non-limiting examples of suitable polysaccharides include starch, glycogen, cellulose, chitin, and combinations thereof. Other sources of carbohydrates include cane juice and molasses.

In general, polysaccharides are first converted into monosaccharides and oligosaccharides by chemical means or by enzymatic methods before they used as a source of carbon for host cells. For instance, cellulose can be converted into glucose by the enzyme cellulase. In certain embodiments, after the breakdown of the polysaccharide, the monosaccharide and/or oligosaccharide constitute at least about 50% by weight of the carbon source as determined at the beginning of the fermentation. In other embodiments, the monosaccharide and/or oligosaccharide constitute at least about 80% or even 90% by weight of the carbon source as determined at the beginning of the fermentation, such that the fermentation medium is essentially free of cellulose.

In certain embodiments, the host cells are exogenously provided ethanol as a carbon source. In other embodiments, the ethanol that is consumed by the host cells as the carbon source was made by the host cells. In other words, the host cells are provided a non-ethanol carbon source (typically a carbohydrate) which is converted by the host cells into ethanol and the ethanol is subsequently consumed by the host cells.

The host cells' use of ethanol can be quantified in a number of ways. In one method, ethanol concentration is used. In addition to being a carbon source, the presence of ethanol in the medium also has the beneficial effects of deterring microbial contaminants.

Thus, in one embodiment, the ethanol concentration in the medium is at least about 1 gram per liter of medium for at least 4 hours. The ethanol concentration can be determined by any method known in the art. It can be measured directly by sampling the medium or indirectly by sampling the offgas. If an indirect method is used such as offgas analysis by mass spectrophotometer, a correlation first be must be established between the offgas measurements in parts per million and the direct measurements of ethanol in the medium. In other embodiments, the ethanol concentration in the medium is between about 1 and about 5 grams, between about 1 and about 10 grams, or between about 1 and about 20 grams per liter of medium. In still other embodiments, the ethanol concentration in the medium is greater than about 10 grams per liter of medium or greater than about 20 grams per liter of medium. In yet other embodiments, the above ethanol concentrations are maintained for at least 6 hours, 8 hours, 10 hours, 12 hours, 24 hours, or 48 hours.

However, host cells can be using ethanol as a carbon source but still have undetectable levels of ethanol or have ethanol concentration close to zero. For example, this can occur when the host cells are consuming ethanol as fast as the ethanol is being supplied. As a result, provided herein are alternative measures for the host cells' ethanol utilization.

In another embodiment, the host cells have a specific ethanol consumption rate of at least 0.01 gram of ethanol per gram of dry cell weight per hour. In other embodiments, the specific ethanol consumption rate is between about 0.01 and about 0.20 gram of ethanol, or between about 0.02 and about 0.10 gram of ethanol per gram of dry cell weight per hour. In still other embodiments, the specific ethanol consumption rate is greater than about 0.10 gram of ethanol per gram of dry cell weight per hour. The specific ethanol consumption rate is maintained for at least 4 hours, 6 hours, 8 hours, 10 hours, 12 hours, 24 hours, or 48 hours.

Alternatively, specific ethanol consumption rate is expressed in terms of grams of ethanol per gram of dry cell weight per day. In some embodiments, the host cells have a specific ethanol consumption rate of at least 0.2 grams of ethanol per gram of dry cell weight per day. In some embodiments, the specific ethanol consumption rate is between about 0.2 and about 5 grams or between about 0.5 and about 3 of ethanol per gram of dry cell weight per day. In other embodiments, the specific ethanol consumption rate is greater than about 3 grams of ethanol per gram of dry cell weight per day.

In certain embodiments, the cells are cultured or maintained under conditions that are not limited by oxygen. In other words, the cells are under aerobic conditions.

However, maintaining fully aerobic conditions can be challenging particularly in large scale processes oxygen due to limitations of mass transfer and the relatively low solubility of oxygen in aqueous solutions. For example, if air is used to sparge into tanks, the solubility of oxygen in water is 9 milligrams per liter at 20° C. If pure oxygen is used instead of air, then the solubility increases to 43 milligrams per liter. In either case (sparging air or pure oxygen), this amount of oxygen is depleted in seconds by an active and concentrated microbial population unless oxygen is continuosly supplied. In comparison, the amounts of other nutrients that are used by the cells during the same period (seconds, e.g., less than a minute) are neglible compared to the bulk concentrations.

We have found that the host cells provided herein are able to tolerate some period of oxygen limitation is and still make high levels of isoprenoid compounds. This flexibility allows for a more economical process by providing savings in terms of tank design, decreased demain for oxygen gas, lower energy costs for aeration and the like. Moreover, under certain circumstances, oxygen limitation appears to be beneficial. Without being bound by theory, cell growth under oxygen limited conditions appears to allow more of the carbon to be directed to product instead of biomass or loss through carbon dioxide.

As a consequence, in certain other embodiments, the host cells are cultured or maintained under conditions that are oxygen limited. The periods of oxygen limitation include at least 4 hours, at least 6 hours, at least 8 hours, at least 10 hours, at least 12 hours, at least 24 hours, or at least 48 hours.

Oxygen limitation occurs when the specific growth rate of the host cells is less than the maximum specific growth rate where oxygen is not limiting (e.g., provided in excess). Specific growth rate is the rate of growth of cells per unit of biomass per unit time and has the units of reciprocal time (1/t). The maximum specific growth rate for cells in a culture medium relates to the effect of a substrate concentration on growth rate which in this case is oxygen. Generally, cells will grow slowly at a low level of the substrate, and as the level of the substrate in the medium increases, so does the rate of cell growth. However, the rate of cell growth does not continue to rise indefinitely, and at high levels of substrate, a given increase in the amount of substrate will produce a smaller and smaller increase in the rate of cell growth. Therefore, the growth rate ultimately reaches a limit, which is often referred to as the maximum specific growth rate.

A theoretical treatment of the relationship between growth rates in culture is well known to those skilled in the art, and is referred to as the Monod equation. See, for example, Pirt, Principles of Microbe and Cell Cultivation, Wiley, NY, 1975, pages 4-10. In this theoretical treatment, the maximum specific rate is an asymptotic limit that is never reached until an infinite level of substrate is reached. In practice, however, the maximum specific growth rate can be considered as being obtained when the conditions under investigation (e.g., a substrate level such as oxygen) support the fastest initial growth rate. For instance, in a fed-batch reactor, the initial condition where all substrates required for growth (e.g. nutrients and oxygen) are supplied in excess and fermentation occurs at the optimal temperature for the host cell is treated as the conditions for the maximum growth rate. See, for example, Lee et al. (1996) Trends Biotechnol. 14:98-105 and Korz et al. (1995) J Biotechnology 39:59-65. Maximum specific growth rate is also sometimes referred to as unlimited growth.

In one method, oxygen limitation is quantified by oxygen concentration in the medium and is expressed in terms of dissolved oxygen concentration (DOC). The DOC in the culture medium can be less than about 20%, less than about 15%, less than about 10%, and less than about 5%. In other embodiments the DOC is about 0% or below the level of detection.

However, because oxygen is consumed by the cells relatively rapidly, a DOC of zero can mean that the cellls are being cultured under anaerobic conditions (no oxygen) or that the cells are consuming oxygen as fast as it is being supplied. In another method, the cells' use of oxygen is expressed in terms of oxygen uptake rate (OUR; the cells' rate of oxygen consumption per liter of medium) to differentiate between these two possibilities. Suitable oxygen uptake rates include less than about 50 mmoles, less than about 40 mmoles, less than about 30 mmoles, less than about 20 mmoles per liter of medium, or less than about 10 mmoles per liter of medium.

Alternatively, specific oxygen uptake rate (SOUR which is OUR divided by cell density) can be used when normalized values with respect to cell densities is preferred. The amount of microorganism per liter of fermentation, or the density of microorganism, can be measured by measuring the weight of microorganism isolated from a given volume of the fermentation medium. A common measure is the dry weight of cells per liter of fermentation medium. Another method which can be used to monitor the fermentation while it is progressing is by a measurement of the optical density of the medium. A common method is to measure the optical density at a wavelength of 600 nm, referred to the OD₆₀₀, or the OD. The OD can be correlated to a the density of a specific type of organism within a specific medium, but the specific relationship between OD and amount of microorganism per volume will not generally be applicable across all types of organisms in all types of media. A calibration curve can be created by measuring the OD and the dry cell weight over a range of cell densities. In some cases, these correlations can be used in different fermentation of the same or similar microorganisms in the same or similar media. Suitable specific oxygen uptake rates include less than about 30 mmoles, less than about 25 mmoles, less than about 20 mmoles, less than about 15 mmoles, less than about 10 mmoles, or less than about 5 mmoles per gram of dry cell weight per hour.

We have also found that the host cells provided herein are able to tolerate some period of phosphate limitation and still make high levels of isoprenoid compounds. Without being bound by theory, cell growth under phosphate limited conditions appears to allow more of the carbon to be directed to product instead of biomass. Suitable concentrations of phosphate in the medium is less than about 5 grams, less than about 4 grams, less than about 3 grams, less than about 2 grams, or less than about 1 gram per liter of medium. In certain embodiments, the phosphate concentration is zero or below the level of detection. The periods of such phosphate limitation include at least 4 hours, at least 6 hours, at least 8 hours, at least 10 hours, at least 12 hours, at least 24 hours, or at least 48 hours.

Host cells can be grown under non-limiting conditions (allowing for maximum specific growth) to build sufficent biomass before limiting conditions (oxygen limted, phosphate limited, or both) are imposed. These limiting conditions include those such that specific growth is less than about 90%, 80%, 75%, 60%, 50%, 40%, 30%, 25%, 20%, 10%, 5%, or 1%, of the maximum specific growth rate.

Although specific embodiments are provided herein, the foregoing description is intended to illustrate and not limit the scope of the embodiments. Other aspects, advantages, and modifications within the scope of the embodiments will be apparent to those skilled in the art.

### EXAMPLES

Unless otherwise indicated, conventional techniques of the biosynthetic industry and the like, which are within the skill of the art, may be used to practice the embodiments provided herein. To the extent such techniques are not described fully herein, one can find ample reference to them in the scientific literature.

In the following examples, efforts have been made to ensure accuracy with respect to numbers used (for example, amounts, temperature, and so on), but variation and deviation can be accommodated, and in the event a clerical error in the numbers reported herein exists, one of ordinary skill in the art can deduce the correct amount in view of the remaining disclosure herein. Unless indicated otherwise, temperature is reported in degrees Celsius, and pressure is at or near atmospheric pressure at sea level. All reagents, unless otherwise indicated, were obtained commercially. The following examples are intended for illustrative purposes only and do not limit in any way the scope of the embodiments provided herein.

### Example 1

This example describes methods for making vectors for the targeted integration of nucleic acids encoding enzymes including enzymes of the MEV pathway into specific chromosomal locations of Saccharomyces cerevisiae.

Genomic DNA was isolated from Saccharomyces cerevisiae strains Y002 and Y003 (CEN.PK2 background MATA or MATα ura3-52 trp1-289 leu2-3,112 his3Δ1 MAL2-8C SUC2) (van Dijken et al. (2000) Enzyme Microb. Technol. 26:706-714), Y007 (S288C background MATA trp1Δ63) (ATCC number 200873), and EG123 (MATA ura3 trp1 leu2 his4 can1) (Michaelis & Herskowitz. (1988) Mol. Cell. Biol. 8:1309-1318). The strains were grown overnight in liquid medium containing 1% Yeast extract, 2% Bacto-peptone, and 2% Dextrose (YPD medium). Cells were isolated from 10 mL liquid cultures by centrifugation at 3,100 rpm, washing of cell pellets in 10 mL ultra-pure water, and re-centrifugation. Genomic DNA was extracted using the Y-DER yeast DNA extraction kit (Pierce Biotechnologies, Rockford, IL) as per manufacturer's suggested protocol. Extracted genomic DNA was re-suspended in 100 uL 10 mM Tris-Cl, pH 8.5, and OD_{260/280} readings were taken on a ND-1000 spectrophotometer (NanoDrop Technologies, Wilmington, DE) to determine genomic DNA concentration and purity.

DNA amplification by Polymerase Chain Reaction (PCR) was done in an Applied Biosystems 2720 Thermocycler (Applied Biosystems Inc., Foster City, CA) using the Phusion High Fidelity DNA Polymerase system (Finnzymes OY, Espoo, Finland) as per manufacturer's suggested protocol. Upon completion of a PCR amplification of a DNA fragment that was to be inserted into the TOPO TA pCR2.1 cloning vector (Invitrogen, Carlsbad, CA), A nucleotide overhangs were created by adding 1 uL of Qiagen Taq Polymerase (Qiagen, Valencia, CA) to the reaction mixture and performing an additional 10 minute, 72° C. PCR extension step, followed by cooling to 4° C. Upon completion of a PCR amplification, 8 uL of a 50% glycerol solution was added to the reaction mix.

Agarose gel electrophoresis was performed using a 1% TBE (0.89 M Tris, 0.89 M boric acid, 0.02 M EDTA sodium salt) agarose gel containing 0.5 ug/mL ethidium bromide, at 120 V, 400 mA for 30 minutes. DNA bands were visualized using ultraviolet light. DNA bands were excised from the gel with a sterile razor blade, and the excised DNA was gel purified using the Zymoclean Gel DNA Recovery Kit (Zymo Research, Orange, CA) according to manufacturer's suggested protocols. The purified DNA was eluted into 10 uL ultra-pure water, and OD_{260/280} readings were taken on a ND-1000 spectrophotometer to determine DNA concentration and purity.

Ligations were performed using 100-500 ug of purified PCR product and High Concentration T4 DNA Ligase (New England Biolabs, Ipswich, MA) as per manufacturer's suggested protocol. For plasmid propagation, ligated constructs were transformed into Escherichia coli DH5α chemically competent cells (Invitrogen, Carlsbad, CA) as per manufacturer's suggested protocol. Positive transformants were selected on solid media containing 1.5% Bacto Agar, 1% Tryptone, 0.5% Yeast Extract, 1% NaCl, and an appropriate antibiotic. Isolated transformants were grown for 16 hours in liquid Luria-Bertoni (LB) medium containing appropriate antibiotics at 37° C., and plasmid was isolated and purified using a QIAprep Spin Miniprep kit (Qiagen, Valencia, CA) as per manufacturer's suggested protocol. Constructs were verified by performing diagnostic restriction enzyme digestions, resolving DNA fragments on an agarose gel, and visualizing the bands using ultraviolet light. Select constructs were also verified by DNA sequencing, which was done by Elim Biopharmaceuticals Inc. (Hayward, CA).

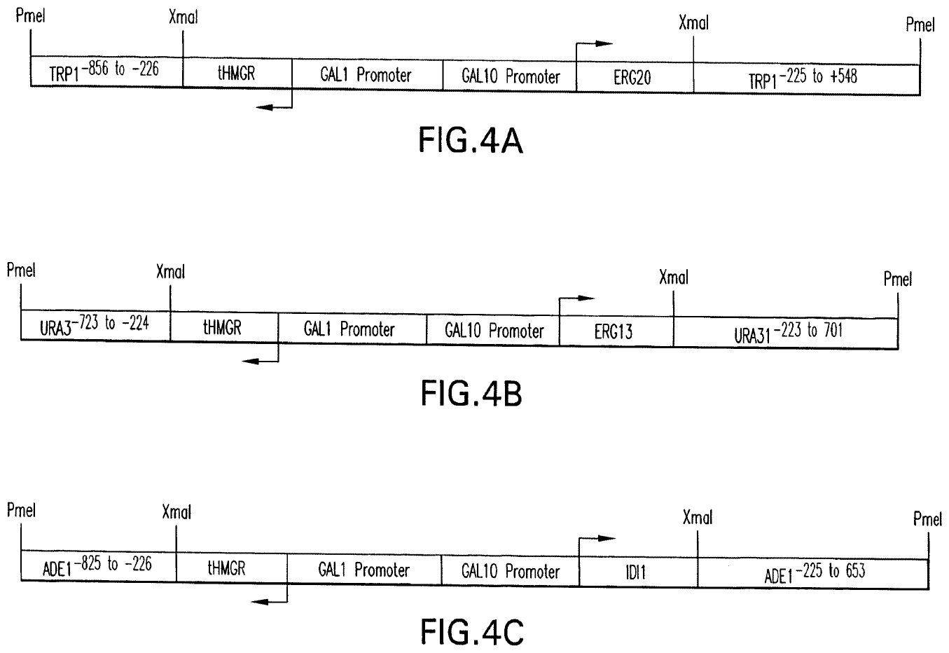

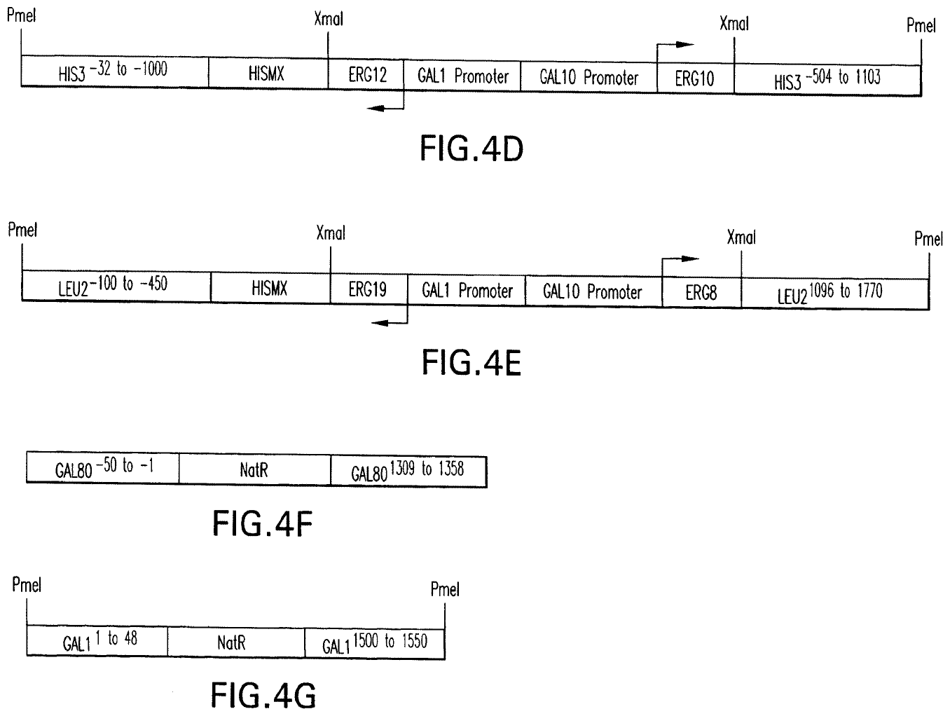

Plasmid pAM489 was generated by inserting the ERG20-P_{GAL}-HMGR insert of vector pAM471 into vector pAM466. Vector pAM471 was generated by inserting DNA fragment ERG20-P_{GAL}-tHMGR, which comprises the open reading frame (ORF) of the ERG20 gene of Saccharomyces cerevisiae (ERG20 nucleotide positions 1 to 1208; A of ATG start codon is nucleotide 1) (ERG20), the genomic locus containing the divergent GAL1 and GAL10 promoter of Saccharomyces cerevisiae (GAL1 nucleotide position −1 to −668) (P_{GAL}), and a truncated ORF of the HMG1 gene of Saccharomyces cerevisiae (HMG1 nucleotide positions 1586 to 3323) (tHMGR), into the TOPO Zero Blunt II cloning vector (Invitrogen, Carlsbad, CA). Vector pAM466 was generated by inserting DNA fragment TRP1^{-856 to +548}, which comprises a segment of the wild-type TRP1 locus of Saccharomyces cerevisiae that extends from nucleotide position −856 to position 548 and harbors a non-native internal XmaI restriction site between bases −226 and −225, into the TOPO TA pCR2.1 cloning vector (Invitrogen, Carlsbad, CA). DNA fragments ERG20-P_{GAL}-tHMGR and TRP1^{-856 to +548} were generated by PCR amplification as outlined in Table 1. For the construction of pAM489, 400 ng of pAM471 and 100 ng of pAM466 were digested to completion using XmaI restriction enzyme (New England Biolabs, Ipswich, MA), DNA fragments corresponding to the ERG20-P_{GAL}-tHMGR insert and the linearized pAM466 vector were gel purified, and 4 molar equivalents of the purified insert was ligated with 1 molar equivalent of the purified linearized vector, yielding pAM489. FIG. 4A shows a map of the ERG20-P_{GAL}-tHMGR insert, and SEQ ID NO: 1 shows the nucleotide sequence of the insert with flanking TRP1 sequences.

TABLE 1
PCR amplifications performed to generate pAM489

| PCR | | | | |
|---|---|---|---|---|
| Round | Template | Primer 1 | Primer 2 | PCR Product |
| 1 | 100 ng of Y003 genomic DNA | 61-67-CPK001-G | 61-67-CPK002-G | TRP1⁻⁸⁵⁶ ^{to -226} |
| | | (SEQ ID NO: 12) | (SEQ ID NO: 13) | |
| | | 61-67-CPK003-G | 61-67-CPK004-G | TRP1^{-225-to} ⁺⁵⁴⁸ |
| | | (SEQ ID NO: 14) | (SEQ ID NO: 15) | |
| | 100 ng of EG123 genomic DNA | 61-67-CPK025-G | 61-67-CPK050-G | ERG20 |
| | | (SEQ ID NO: 36) | (SEQ ID NO: 44) | |
| | 100 ng of Y002 genomic DNA | 61-67-CPK051-G | 61-67-CPK052-G | PGAL |
| | | (SEQ ID NO: 45) | (SEQ ID NO: 46) | |
| | | 61-67-CPK053-G | 61-67-CPK031-G | tHMGR |
| | | (SEQ ID NO: 47) | (SEQ ID NO: 37) | |
| 2 | 100 ng each of TRP1⁻⁸⁵⁶ ^{to -226} | 61-67-CPK001-G | 61-67-CPK004-G | TRP1⁻⁸⁵⁶ ^{to +548} |
| | and TRP1^{-225-to} ⁺⁵⁴⁸ purified | (SEQ ID NO: 12) | (SEQ ID NO: 15) | |
| | PCR products | | | |
| | 100 ng each of ERG20 and | 61-67-CPK025-G | 61-67-CPK052-G | ERG20-PGAL |
| | P_{GAL} purified PCR products | (SEQ ID NO: 36) | (SEQ ID NO: 46) | |
| 3 | 100 ng each of ERG20-P_{GAL} | 61-67-CPK025-G | 61-67-CPK031-G | ERG20-P_{GAL}- |
| | and tHMGR purified PCR | (SEQ ID NO: 36) | (SEQ ID NO: 37) | tHMGR |
| | products | | | |

Plasmid pAM491 was generated by inserting the ERG13-P_{GAL}-tHMGR insert of vector pAM472 into vector pAM467. Vector pAM472 was generated by inserting DNA fragment ERG13-P_{GAL}-tHMGR, which comprises the ORF of the ERG13 gene of Saccharomyces cerevisiae (ERG13 nucleotide positions 1 to 1626) (ERG13), the genomic locus containing the divergent GAL1 and GAL10 promoter of Saccharomyces cerevisiae (GAL1 nucleotide position −1 to −668) (P_{GAL}), and a truncated ORF of the HMG1 gene of Saccharomyces cerevisiae (HMG1 nucleotide position 1586 to 3323) (tHMGR), into the TOPO Zero Blunt II cloning vector. Vector pAM467 was generated by inserting DNA fragment URA3^{-723 to 701}, which comprises a segment of the wild-type URA3 locus of Saccharomyces cerevisiae that extends from nucleotide position −723 to position −224 and harbors a non-native internal XmaI restriction site between bases −224 and −223, into the TOPO TA pCR2.1 cloning vector. DNA fragments ERG13-P_{GAL}-tHMGR and URA3^{-723 to 701} were generated by PCR amplification as outlined in Table 2. For the construction of pAM491, 400 ng of pAM472 and 100 ng of pAM467 were digested to completion using XmaI restriction enzyme, DNA fragments corresponding to the ERG13-P_{GAL}-tHMGR insert and the linearized pAM467 vector were gel purified, and 4 molar equivalents of the purified insert was ligated with 1 molar equivalent of the purified linearized vector, yielding pAM491. FIG. 4B shows a map of the ERG13-P_{GAL}-tHMGR insert, and SEQ ID NO: 2 shows the nucleotide sequence of the insert with flanking URA3 sequences.

TABLE 2
PCR amplifications performed to generate pAM491

| PCR | | | | |
|---|---|---|---|---|
| Round | Template | Primer 1 | Primer 2 | PCR Product |
| 1 | 100 ng of Y007 genomic DNA | 61-67-CPK005-G | 61-67-CPK006-G | URA3⁻⁷²³ ^{to -224} |
| | | (SEQ ID NO: 16) | (SEQ ID NO: 17) | |
| | | 61-67-CPK007-G | 61-67-CPK008-G | URA3⁻²²³ ^{to 701} |
| | | (SEQ ID NO: 18) | (SEQ ID NO: 19) | |
| | 100 ng of Y002 genomic DNA | 61-67-CPK032-G | 61-67-CPK054-G | ERG13 |
| | | (SEQ ID NO: 38) | (SEQ ID NO: 48) | |
| | | 61-67-CPK052-G | 61-67-CPK055-G | P_{GAL} |
| | | (SEQ ID NO: 46) | (SEQ ID NO: 49) | |
| | | 61-67-CPK031-G | 61-67-CPK053-G | tHMGR |
| | | (SEQ ID NO: 37) | (SEQ ID NO: 47) | |
| 2 | 100 ng each of URA3⁻⁷²³ ^{to -224} | 61-67-CPK005-G | 61-67-CPK008-G | URA3⁻⁷²³ ^{to 701} |
| | and URA3⁻²²³ ^{to 701} purified | (SEQ ID NO: 16) | (SEQ ID NO: 19) | |
| | PCR products | | | |
| | 100 ng each of ERG13 and | 61-67-CPK032-G | 61-67-CPK052-G | ERG13-P_{GAL} |
| | P_{GAL} purified PCR products | (SEQ ID NO: 38) | (SEQ ID NO: 46) | |
| 3 | 100 ng each of ERG13-P_{GAL} | 61-67-CPK031-G | 61-67-CPK032-G | ERG13-P_{GAL}- |
| | and tHMGR purified PCR | (SEQ ID NO: 37) | (SEQ ID NO: 38) | tHMGR |
| | products | | | |

Plasmid pAM493 was generated by inserting the IDI1-P_{GAL}-tHMGR insert of vector pAM473 into vector pAM468. Vector pAM473 was generated by inserting DNA fragment IDI1-P_{GAL}-tHMGR, which comprises the ORF of the IDII gene of Saccharomyces cerevisiae (IDI1 nucleotide position 1 to 1017) (IDI1), the genomic locus containing the divergent GAL1 and GAL10 promoter of Saccharomyces cerevisiae (GAL1 nucleotide position −1 to −668) (P_{GAL}), and a truncated ORF of the HMG1 gene of Saccharomyces cerevisiae (HMG1 nucleotide positions 1586 to 3323) (tHMGR), into the TOPO Zero Blunt II cloning vector. Vector pAM468 was generated by inserting DNA fragment ADE1^{-825 to 653}, which comprises a segment of the wild-type ADE1 locus of Saccharomyces cerevisiae that extends from nucleotide position −225 to position 653 and harbors a non-native internal XmaI restriction site between bases −226 and −225, into the TOPO TA pCR2.1 cloning vector. DNA fragments IDI1-P_{GAL}-tHMGR and ADE1^{-825 to 653} were generated by PCR amplification as outlined in Table 3. For the construction of pAM493, 400 ng of pAM473 and 100 ng of pAM468 were digested to completion using XmaI restriction enzyme, DNA fragments corresponding to the IDI1-P_{GAL}-tHMGR insert and the linearized pAM468 vector were gel purified, and 4 molar equivalents of the purified insert was ligated with 1 molar equivalent of the purified linearized vector, yielding vector pAM493. FIG. 4C shows a map of the IDI1-P_{GAL}-tHMGR insert, and SEQ ID NO: 3 shows the nucleotide sequence of the insert with flanking ADE1 sequences.

TABLE 3
PCR amplifications performed to generate pAM493

| PCR | | | | |
|---|---|---|---|---|
| Round | Template | Primer 1 | Primer 2 | PCR Product |
| 1 | 100 ng of Y007 genomic DNA | 61-67-CPK009-G | 61-67-CPK010-G | ADE1⁻⁸²⁵ ^{to -226} |
| | | (SEQ ID NO: 20) | (SEQ ID NO: 21) | |
| | | 61-67-CPK011-G | 61-67-CPK012-G | ADE1⁻²²⁵ ^{to 653} |
| | | (SEQ ID NO: 22) | (SEQ ID NO: 23) | |
| | 100 ng of Y002 genomic DNA | 61-67-CPK047-G | 61-67-CPK064-G | IDI1 |
| | | (SEQ ID NO: 43) | (SEQ ID NO: 58) | |
| | | 61-67-CPK052-G | 61-67-CPK065-G | P_{GAL} |
| | | (SEQ ID NO: 46) | (SEQ ID NO: 59) | |
| | | 61-67-CPK031-G | 61-67-CPK053-G | tHMGR |
| | | (SEQ ID NO: 37) | (SEQ ID NO: 47) | |
| 2 | 100 ng each of ADE1⁻⁸²⁵ ^{to -226} | 61-67-CPK009-G | 61-67-CPK012-G | ADE1⁻⁸²⁵ ^{to 653} |
| | and ADE1⁻²²⁵ ^{to 653} purified | (SEQ ID NO: 20) | (SEQ ID NO: 23) | |
| | PCR products | | | |
| | 100 ng each of IDI1 and P_{GAL} | 61-67-CPK047-G | 61-67-CPK052-G | IDI1-P_{GAL} |
| | purified PCR products | (SEQ ID NO: 43) | (SEQ ID NO: 46) | |
| 3 | 100 ng each of IDI1-P_{GAL} and | 61-67-CPK031-G | 61-67-CPK047-G | IDI1-P_{GAL}- |
| | tHMGR purified PCR products | (SEQ ID NO: 37) | (SEQ ID NO: 43) | tHMGR |

Plasmid pAM495 was generated by inserting the ERG10-P_{GAL}-ERG12 insert of pAM474 into vector pAM469. Vector pAM474 was generated by inserting DNA fragment ERG10-P_{GAL}-ERG12, which comprises the ORF of the ERG10 gene of Saccharomyces cerevisiae (ERG 10 nucleotide position 1 to 1347) (ERG10), the genomic locus containing the divergent GAL1 and GAL 10 promoter of Saccharomyces cerevisiae (GAL1 nucleotide position −1 to −668) (P_{GAL}), and the ORF of the ERG12 gene of Saccharomyces cerevisiae (ERG12 nucleotide position 1 to 1482) (ERG12), into the TOPO Zero Blunt II cloning vector. Vector pAM469 was generated by inserting DNA fragment HIS3^{-32 to -1000}-HISMX-HIS3^{504 to -1103}, which comprises two segments of the HIS locus of Saccharomyces cerevisiae that extend from nucleotide position −32 to position −1000 and from nucleotide position 504 to position 1103, a HISMX marker, and a non-native XmaI restriction site between the HIS3^{504 to -1103} sequence and the HISMX marker, into the TOPO TA pCR2.1 cloning vector. DNA fragments ERG10-P_{GAL}-ERG12 and HIS3^{-32 to -1000}-HISMX-HIS3^{504 to -1103} were generated by PCR amplification as outlined in Table 4. For construction of pAM495, 400 ng of pAM474 and 100 ng of pAM469 were digested to completion using XmaI restriction enzyme, DNA fragments corresponding to the ERG10-P_{GAL}-ERG12 insert and the linearized pAM469 vector were gel purified, and 4 molar equivalents of the purified insert was ligated with 1 molar equivalent of the purified linearized vector, yielding vector pAM495. FIG. 4D shows a map of the ERG10-P_{GAL}-ERG12 insert, and SEQ ID NO: 4 shows the nucleotide sequence of the insert with flanking HIS3 sequences.

TABLE 4
PCR reactions performed to generate pAM495

| PCR | | | | |
|---|---|---|---|---|
| Round | Template | Primer 1 | Primer 2 | PCR Product |
| 1 | 100 ng of Y007 genomic DNA | 61-67-CPK013-G | 61-67-CPK014alt-G | HIS3⁻³² ^{to -1000} |
| | | (SEQ ID NO: 24) | (SEQ ID NO: 25) | |
| | | 61-67-CPK017-G | 61-67-CPK018-G | HIS3^{504 to -1103} |
| | | (SEQ ID NO: 28) | (SEQ ID NO: 29) | |
| | | 61-67-CPK035-G | 61-67-CPK056-G | ERG10 |
| | | (SEQ ID NO: 39) | (SEQ ID NO: 50) | |
| | | 61-67-CPK057-G | 61-67-CPK058-G | P_{GAL} |
| | | (SEQ ID NO: 51) | (SEQ ID NO: 52) | |
| | | 61-67-CPK040-G | 61-67-CPK059-G | ERG12 |
| | | (SEQ ID NO: 40) | (SEQ ID NO: 53) | |
| | 10 ng of plasmid pAM330 | 61-67-CPK015alt-G | 61-67-CPK016-G | HISMX |
| | DNA ** | (SEQ ID NO: 26) | (SEQ ID NO: 27) | |
| 2 | 100 ng each of HIS3^{504 to -1103} | 61-67-CPK015alt-G | 61-67-CPK018-G | HISMX-HIS3^{504 to -1103} |
| | and HISMX PCR | (SEQ ID NO: 26) | (SEQ ID NO: 29) | |
| | purified products | | | |
| | 100 ng each of ERG10 and | 61-67-CPK035-G | 61-67-CPK058-G | ERG10-P_{GAL} |
| | P_{GAL} purified PCR products | (SEQ ID NO: 39) | (SEQ ID NO: 52) | |
| 3 | 100 ng each of HIS3⁻³² ^{to -1000} | 61-67-CPK013-G | 61-67-CPK018-G | HIS3⁻³² ^{to -1000}- |
| | and HISMX-HIS3^{504 to -1103} | (SEQ ID NO: 24) | (SEQ ID NO: 29) | HISMX-HIS3^{504 to -1103} |
| | purified PCR products | | | |
| | 100 ng each of ERG10- | 61-67-CPK035-G | 61-67-CPK040-G | ERG10-P_{GAL}- |
| | P_{GAL} and ERG12 purified | (SEQ ID NO: 39) | (SEQ ID NO: 40) | ERG12 |
| | PCR products | | | |

** The HISMX marker in pAM330 originated from pFA6a-HISMX6-PGALI as described by van Dijken et al. ((2000) Enzyme Microb. Technol. 26(9-10):706-714).

Plasmid pAM497 was generated by inserting the ERG8-P_{GAL}-ERG19 insert of pAM475 into vector pAM470. Vector pAM475 was generated by inserting DNA fragment ERG8-P_{GAL}-ERG19, which comprises the ORF of the ERG8 gene of Saccharomyces cerevisiae (ERG8 nucleotide position 1 to 1512) (ERG8), the genomic locus containing the divergent GAL1 and GAL 10 promoter of Saccharomyces cerevisiae (GAL1 nucleotide position −1 to −668) (P_{GAL}), and the ORF of the ERG19 gene of Saccharomyces cerevisiae (ERG19 nucleotide position 1 to 1341) (ERG19), into the TOPO Zero Blunt II cloning vector. Vector pAM470 was generated by inserting DNA fragment LEU2^{-100 to 450}-HISMX-LEU2^{1096 to 1770}, which comprises two segments of the LEU2 locus of Saccharomyces cerevisiae that extend from nucleotide position −100 to position 450 and from nucleotide position 1096 to position 1770, a HISMX marker, and a non-native XmaI restriction site between the LEU2^{1096 to 1770} sequence and the HISMX marker, into the TOPO TA pCR2.1 cloning vector. DNA fragments ERG8-P_{GAL}-ERG19 and LEU2^{-100 to 450}-HISMX-LEU2^{1096 to 1770} were generated by PCR amplification as outlined in Table 5. For the construction of pAM497, 400 ng of pAM475 and 100 ng of pAM470 were digested to completion using XmaI restriction enzyme, DNA fragments corresponding to the ERG8-P_{GAL}-ERG19 insert and the linearized pAM470 vector were purified, and 4 molar equivalents of the purified insert was ligated with 1 molar equivalent of the purified linearized vector, yielding vector pAM497. FIG. 4E for a map of the ERG8-P_{GAL}-ERG19 insert, and SEQ ID NO: 5 shows the nucleotide sequence of the insert with flanking LEU2 sequences.

TABLE 5
PCR reactions performed to generate pAM497

| PCR | | | | |
|---|---|---|---|---|
| Round | Template | Primer 1 | Primer 2 | PCR Product |
| 1 | 100 ng of Y007 genomic DNA | 61-67-CPK019-G | 61-67-CPK020-G | LEU2^{-100 to 450} |
| | | (SEQ ID NO: 30) | (SEQ ID NO: 31) | |
| | | 61-67-CPK023-G | 61-67-CPK024-G | LEU2^{1096 to 1770} |
| | | (SEQ ID NO: 34) | (SEQ ID NO: 35) | |
| | 10 ng of plasmid pAM330 DNA ** | 61-67-CPK021-G | 61-67-CPK022-G | HISMX |
| | | (SEQ ID NO: 32) | (SEQ ID NO: 33) | |
| | 100 ng of Y002 genomic DNA | 61-67-CPK041-G | 61-67-CPK060-G | ERG8 |
| | | (SEQ ID NO: 41) | (SEQ ID NO: 54) | |
| | | 61-67-CPK061-G | 61-67-CPK062-G | P_{GAL} |
| | | (SEQ ID NO: 55) | (SEQ ID NO: 56) | |
| | | 61-67-CPK046-G | 61-67-CPK063-G | ERG19 |
| | | (SEQ ID NO: 42) | (SEQ ID NO: 57) | |
| 2 | 100 ng each of LEU2^{1096 to 1770} | 61-67-CPK021-G | 61-67-CPK024-G | HISMX- |
| | and HISMX purified PCR | (SEQ ID NO: 32) | (SEQ ID NO: 35) | LEU2^{1096 to 1770} |
| | products | | | |
| | 100 ng each of ERG8 and P_{GAL} | 61-67-CPK041-G | 61-67-CPK062-G | ERG8-P_{GAL} |
| | purified PCR products | (SEQ ID NO: 41) | (SEQ ID NO: 56) | |
| 3 | 100 ng of LEU2^{-100 to 450} and | 61-67-CPK019-G | 61-67-CPK024-G | LEU2^{-100 to 450}- |
| | HISMX- LEU2^{1096 to 1770} purified | (SEQ ID NO: 30) | (SEQ ID NO: 35) | HISMX- |
| | PCR products | | | LEU2^{1096 to 1770} |
| | 100 ng each of ERG8-P_{GAL} and | 61-67-CPK041-G | 61-67-CPK046-G | ERG8-P_{GAL}- |
| | ERG19 purified PCR products | (SEQ ID NO: 41) | (SEQ ID NO: 42) | ERG19 |

** The HISMX marker in pAM330 originated from pFA6a-HISMX6-PGAL1 as described by van Dijken et al. ((2000) Enzyme Microb. Technol. 26(9-10):706-714).

### Example 2

This example describes methods for making plasmids and DNA fragments useful in the embodiments provided herein.

Plasmid pAM584 was generated by inserting DNA fragment GAL7^{4 to 1021}-HPH-GAL1^{1637 to 2587} into the TOPO ZERO Blunt II cloning vector (Invitrogen, Carlsbad, CA). DNA fragment GAL7^{4 to 1021}-HPH-GAL1^{1637 to 2587} comprises a segment of the ORF of the GAL7 gene of Saccharomyces cerevisiae (GAL7 nucleotide positions 4 to 1021) (GAL7^{4 to 1021}), the hygromycin resistance cassette (HPH), and a segment of the 3′ untranslated region (UTR) of the GAL1 gene of Saccharomyces cerevisiae (GAL1 nucleotide positions 1637 to 2587). The DNA fragment was generated by PCR amplification as outlined in Table 6. SEQ ID NO: 9 is the nucleotide sequence of DNA fragment GAL7^{4 to 1021}-HPH-GAL1^{1637 to 2587}.

TABLE 6
PCR reactions performed to generate pAM584

| PCR | | | | |
|---|---|---|---|---|
| Round | Template | Primer 1 | Primer 2 | PCR Product |
| 1 | 100 ng of Y002 genomic DNA | 91-014-CPK236-G | 91-014-CPK237-G | GAL7^{4 to 1021} |
| | | (SEQ ID NO: 65) | (SEQ ID NO: 66) | |
| | | 91-014-CPK232-G | 91-014-CPK233-G | GAL1^{1637 to 2587} |
| | | (SEQ ID NO: 63) | (SEQ ID NO: 64) | |
| | 10 ng of plasmid pAM547 DNA ** | 91-014-CPK231-G | 91-014-CPK238-G | HPH |
| | | (SEQ ID NO: 62) | (SEQ ID NO: 67) | |
| 2 | 100 ng each of GAL7^{4 to 1021} and | 91-014-CPK231-G | 91-014-CPK236-G | GAL7^{4 to 1021}- |
| | HPH purified PCR products | (SEQ ID NO: 62) | (SEQ ID NO: 65) | HPH |
| 3 | 100 ng of each GAL1^{1637 to 2587} | 91-014-CPK233-G | 91-014-CPK236-G | GAL7^{4 to 1021}- |
| | and GAL7^{4 to 1021}-HPH purified | (SEQ ID NO: 64) | (SEQ ID NO: 65) | HPH- |
| | PCR products | | | GAL1^{1637 to 2587} |

** Plasmid pAM547 was generated synthetically, and comprises the HPH cassette, which consists of the coding sequence for the hygromycin B phosphotransferase of Escherichia coli flanked by the promoter and terminator of the Tef1 gene of Kluyveromyces lactis.

DNA fragment GAL80^{-50 to -1}-NatR-GAL80^{1309 to 1358} was generated by PCR amplification. The DNA fragments includes the nourseothricin resistance selectable marker gene of Streptomyces noursei (NatR) flanked by two segments of 50 nucleotides each that map immediately upstream and immediately downstream of the coding region of the GAL80 gene of Saccharomyces cerevisiae (GAL80 nucleotide position −50 to −1 and 1309 to 1358; GAL80^{-50 to -1} and GAL80^{1309 to 1358}, respectively). FIG. 4F shows a map, and SEQ ID NO: 8 the nucleotide sequence, of DNA fragment GAL80^{-50 to -1}-NatR-GAL80^{1309 to 1358}.

DNA fragment GAL1^{1 to 48}-NatR-GAL1^{1500 to 1550} was generated by PCR amplification. The DNA fragment includes the nourseothricin resistance selectable marker gene of Streptomyces noursei (NatR) flanked by two segments of 40 to 50 nucleotides each that map to the 5′ and the 3′ end of the coding region of the GAL1 gene of Saccharomyces cerevisiae (GAL1 nucleotide position 1 to 48 and 1500 to 1550; GAL1^{1 to 48} and GAL1^{1500 to 1550}, respectively). FIG. 4G shows a map, and SEQ ID NO: 68 the nucleotide sequence of DNA fragment GAL1^{1 to 48}-NatR-GAL1^{1500 to 1550}.

Expression plasmid pAM353 was generated by inserting a nucleotide sequence encoding a β-farnesene synthase into the pRS425-Gal1 vector (Mumberg et. al. (1994) Nucl. Acids. Res. 22 (25): 5767-5768). The nucleotide sequence insert was generated synthetically, using as a template the coding sequence of the β-farnesene synthase gene of Artemisia annua (GenBank accession number AY835398) codon-optimized for expression in Saccharomyces cerevisiae (SEQ ID NO: 10). The synthetically generated nucleotide sequence was flanked by 5′ BamHI and 3′ XhoI restriction sites, and could thus be cloned into compatible restriction sites of a cloning vector such as a standard pUC or pACYC origin vector. The synthetically generated nucleotide sequence was isolated by digesting to completion the DNA synthesis construct using BamHI and XhoI restriction enzymes. The reaction mixture was resolved by gel electrophoresis, the approximately 1.7 kb DNA fragment comprising the β-farnesene synthase coding sequence was gel extracted, and the isolated DNA fragment was ligated into the BamHI XhoI restriction site of the pRS425-Gal1 vector, yielding expression plasmid pAM353.

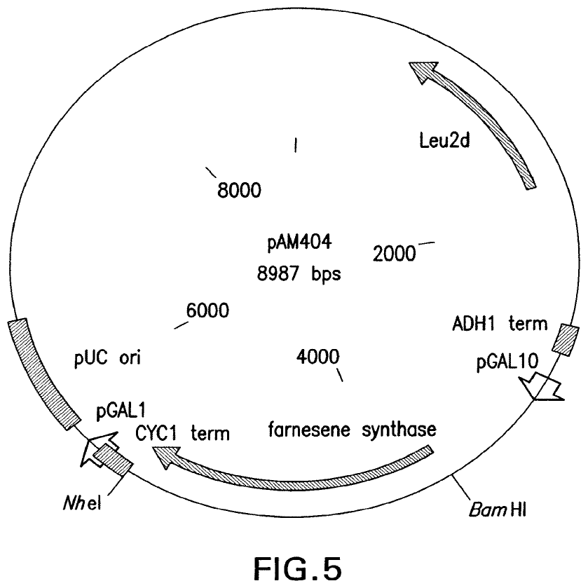

Expression plasmid pAM404 was generated by inserting a nucleotide sequence encoding the β-farnesene synthase of Artemisia annua, codon-optimized for expression in Saccharomyces cerevisiae, into vector pAM178 (SEQ ID NO: 69). The nucleotide sequence encoding the β-farnesene synthase was PCR amplified from pAM353 using primers 52-84 pAM326 BamHI (SEQ ID NO: 71) and 52-84 pAM326 NheI (SEQ ID NO: 72). The resulting PCR product was digested to completion using BamHI and NheI restriction enzymes, the reaction mixture was resolved by gel electrophoresis, the approximately 1.7 kb DNA fragment comprising the β-farnesene synthase coding sequence was gel extracted, and the isolated DNA fragment was ligated into the BamHI NheI restriction site of vector pAM178, yielding expression plasmid pAM404 (see FIG. 5 for a plasmid map).

### Example 3

This example describes the generation of Saccharomyces cerevisiae strains useful in the embodiments provided herein.

Saccharomyces cerevisiae strains CEN.PK2-1C Y002 and Y003 (MATA or MATalpha; ura3-52; trp1-289; leu2-3,112; his3Δ1; MAL2-8C; SUC2) (van Dijken et al. (2000) Enzyme Microb. Technol. 26 (9-10): 706-714) were prepared for introduction of inducible MEV pathway genes by replacing the ERG9 promoter with the Saccharomyces cerevisiae MET3 promoter, and the ADE1 ORF with the Candida glabrata LEU2 gene (CgLEU/2). This was done by PCR amplifying the KanMX-P_{MET3} region of vector pAM328 (SEQ ID NO: 6), which comprises the P_{MET3} promoter preceded by the kanamycin resistance marker flanked by the promoter and terminator of the Tef1 gene of Kluyveromyces lactis, using primers 50-56-pw100-G (SEQ ID NO: 10) and 50-56-pw101-G (SEQ ID NO: 11), which include 45 base pairs of homology to the native ERG9 promoter, transforming 10 ug of the resulting PCR product into exponentially growing Y002 and Y003 cells using 40% w/w Polyethelene Glycol 3350 (Sigma-Aldrich, St. Louis, MO), 100 mM Lithium Acetate (Sigma-Aldrich, St. Louis, MO), and 10 ug Salmon Sperm DNA (Invitrogen Corp., Carlsbad, CA), and incubating the cells at 30° C. for 30 minutes followed by heat shocking them at 42° C. for 30 minutes (Schiestl and Gietz (1989) Curr. Genet. 16:339-346). Positive recombinants were identified by their ability to grow on rich medium containing 0.5 ug/mL Geneticin (Invitrogen Corp., Carlsbad, CA), and selected colonies were confirmed by diagnostic PCR. The resultant clones were given the designation Y93 (MAT A) and Y94 (MAT alpha). The 3.5 kb CgLEU2 genomic locus was then amplified from Candida glabrata genomic DNA (ATCC, Manassas, VA) using primers 61-67-CPK066-G (SEQ ID NO: 60) and 61-67-CPK067-G (SEQ ID NO: 61), which contain 50 base pairs of flanking homology to the ADE1 ORF, and 10 ug of the resulting PCR product were transformed into exponentially growing Y93 and Y94 cells, positive recombinants were selected for growth in the absence of leucine supplementation, and selected clones were confirmed by diagnostic PCR. The resultant clones were given the designation Y176 (MAT A) and Y177 (MAT alpha).

Strain Y188 was generated by digesting pAM491 and pAM495 plasmid DNA to completion using PmeI restriction enzyme (New England Biolabs, Beverly, MA), and introducing the purified DNA inserts into exponentially growing Y176 cells. Positive recombinants were selected for by growth on medium lacking uracil and histidine, and integration into the correct genomic locus was confirmed by diagnostic PCR.

Strain Y189 was generated by digesting pAM489 and pAM497 plasmid DNA to completion using PmeI restriction enzyme, and introducing the purified DNA inserts into exponentially growing Y177 cells. Positive recombinants were selected for by growth on medium lacking tryptophan and histidine, and integration into the correct genomic locus was confirmed by diagnostic PCR.

Approximately 1×10⁷ cells from strains Y188 and Y189 were mixed on a YPD medium plate for 6 hours at room temperature to allow for mating. The mixed cell culture was plated to medium lacking histidine, uracil, and tryptophan to select for growth of diploid cells. Strain Y238 was generated by transforming the diploid cells using pAM493 plasmid DNA that had been digested to completion using PmeI restriction enzyme, and introducing the purified DNA insert into the exponentially growing diploid cells. Positive recombinants were selected for by growth on medium lacking adenine, and integration into the correct genomic locus was confirmed by diagnostic PCR.

Haploid strain Y211 (MAT alpha) was generated by sporulating strain Y238 in 2% potassium acetate and 0.02% Raffinose liquid medium, isolating approximately 200 genetic tetrads using a Singer Instruments MSM300 series micromanipulator (Singer Instrument LTD, Somerset, UK), identifying independent genetic isolates containing the appropriate complement of introduced genetic material by their ability to grow in the absence of adenine, histidine, uracil, and tryptophan, and confirming the integration of all introduced DNA by diagnostic PCR.

Strain Y227 was generated from strain Y211 by rendering the strain capable of converting FPP to amorpha-4,11-diene. To this end, exponentially growing Y211 cells were transformed with expression plasmid pAM426 (SEQ ID NO: 7), which comprises a GAL1 promoter operably linked to the coding sequence of an amorpha-4,11-diene synthase gene that is codon-optimized for expression in Saccharomyces cerevisiae (Merke et al. (2000) Ach. Biochem. Biophys. 381:173-180). Host cell transformants were selected on complete synthetic defined media lacking leucine.

Strain Y293 was generated from strain Y227 by deleting the coding sequence of the GAL80 gene, and thus rendering the GAL promoters in the strain constitutively active. To this end, exponentially growing Y227 cells were transformed with DNA fragment GAL80^{-50 to -1}-NatR-GAL80^{1309 to 1358}. Host cell transformants were selected on YPD agar containing 100 μg/mL nourseothricin, single colonies were picked, and integration into the correct genomic locus was confirmed by diagnostic PCR.

Strain Y337 was generated from strain Y227 by rendering the strain unable to catabolize galactose. To this end, pAM584 plasmid DNA was digested to completion using PmeI restriction enzyme, and the purified DNA insert GAL7^{4 to 1021}-HPH-GAL1^{1637 to 2587} was introduced into exponentially growing Y227 cells. Positive recombinants were selected for by growth on YPD agar containing hygromycin B (Sigma, St. Louis, MO). Integration into the correct genomic locus was confirmed by diagnostic PCR and by testing the strain for inability to use galactose as a carbon source.

Strain Y351 was generated from strain Y211 by rendering the strain unable to catabolize galactose. To this end, pAM584 plasmid DNA was digested to completion using PmeI restriction enzyme, and the purified DNA insert GAL7^{4 to 1021}-HPH-GAL1^{1637 to 2587} was introduced into exponentially growing Y211. Host cell transformants were selected on YPD agar containing hygromycin B. Integration into the correct genomic locus was confirmed by diagnostic PCR and by testing the strain for inability to use galactose as a carbon source.

Strain Y352 was generated from strain Y351 by rendering the strain able to produce β-farnesene synthase. To this end, exponentially growing Y351 cells were transformed with expression plasmid pAM404. Host cell transformants were selected on complete synthetic defined media lacking leucine.

Strain Y283 was generated from strain Y227 by deleting the coding sequence of the GAL1 gene and thus rendering the strain unable to catabolize galactose. To this end, exponentially growing Y227 cells were transformed with DNA fragment GAL1^{1 to 48}-NatR-GAL1^{1500 to 1550}. Host cell transformants were selected on YPD agar containing 100 μg/mL nourseothricin, single colonies were picked, and integration into the correct genomic locus was confirmed by diagnostic PCR and by growing the strain on agar containing glycerol and 2-deoxygalactose (a functional GAL1p would convert the latter into a toxin).

Strain Y221 was generated from strain Y211 by transforming exponentially growing Y211 cells with vector pAM178 (SEQ ID NO: 69). Positive transformants were selected for by growth on complete synthetic medium lacking leucine.

Strain Y290 was generated from strain Y221 by deleting the coding sequence of the GAL80 gene, and thus rendering the GAL promoters in the strain constitutively active.

Strain Y318 was generated from strain Y290 by screening colonies for loss of the pAM178 vector.

Strain 409 was generated from strain Y318 by rendering the strain able to produce β-farnesene synthase in the presence of galactose. To this end, exponentially growing Y318 cells were transformed with expression plasmid pAM404. Host cell transformants were selected on complete synthetic defined media lacking leucine.

Strain Y419 was generated from strain Y409 by rendering the GAL promoters in the strain constitutively active and able to express higher levels of GAL4p in the presence of glucose (i.e., able to more efficiently drive expression off galactose-inducible promoters in the presence of glucose, as well as assure that there is enough Gal4p transcription factor to drive expression from all the galactose-inducible promoters in the cell). To this end, the KanMX marker at the ERG9 locus in strain Y409 was replaced by a DNA fragment that comprised the ORF of the GAL4 gene of Saccharomyces cerevisiae under the control of an “operative constitutive” version of its native promoter (Griggs & Johnston (1991) PNAS 88 (19):8597-8601) and the GAL4 terminator (P_{Gal4OC}-GAL4-T_{GAL4}), and the nourseothricin resistance selectable marker gene of Streptomyces noursei (NatR) flanked by the promoter and terminator of the Tef1 gene of Kluyveromyces lactis.

Strain Y677 was generated from strain Y419 by introducing another copy of the coding region of mevalonate kinase under the control of P_{GAL1} at the GAL80 locus.

Cell banks of strains Y293, Y283, Y352 and Y677 were prepared by growing the cells in seed medium at 30° C. until they reached an OD₆₀₀ of between 2 to 5. At that time, the flasks were placed on ice. Three parts culture and 2 parts ice cold sterile 50% glycerol were combined, and 1 mL aliquots of this mixture were frozen at −80° C. in cyrovials. The same procedure was used for strain Y337, however the OD₆₀₀ for that strain was 13.6 at the time it was frozen.

### Example 4

This example describes the production of amorpha-4,11-diene by host cells in fed batch, carbon-restricted fermentation with a glucose only feed.

Y337 seed cultures were prepared by inoculating a 1 mL frozen vial into a 250 mL flask containing 50 mL seed medium (Table 7). After ˜24 hours of growth at 30° C., 0.5 mL of the culture was sub-cultured into additional 250 mL flasks each containing 50 mL seed medium. The seed cultures were grown at 30° C. overnight to an OD₆₀₀ of approximately 3 to 12. Flasks were pooled and used to inoculate bioreactors containing batch medium (Table 8) at 10% v/v.

TABLE 7
Seed medium

| Component | Seed Medium |
|---|---|
| tap water (mL/L) | 350 |
| 2 × batch base (mL/L) ^{a)} | 500 |
| 715 g/L glucose monohydrate (mL/L) ^{b)} | 30 |
| Yeast vitamin solution (mL/L) (Table 9) | 12 |
| Yeast trace metals solution (mL/L) (Table 9) | 10 |
| succinate (0.5M, pH 5.0) (mL/L) ^{c)} | 100 |

^{a)} 16 g/L KH₂PO₄, 30 g/L (NH₄) ₂SO₄, and 12.3 g/L MgSO₄*7H₂O (Note: no heating while mixing these components)
^{b)} The glucose monohydrate stock solution was prepared by dissolving the sugar in water with heating, allowing the solution to cool, and filter sterilizing.
^{c)} The succinate stock solution was prepared by dissolving succinic acid in water with heating, letting the solution cool, adjusting the pH to 5.05 with NaOH, and sterilizing the solution by autoclaving (45 minutes at 121° C.).

TABLE 8
Bioreactor batch medium

| Component | Batch Medium |
|---|---|
| tap water (mL/L) | 350 |
| 2 × batch base (mL/L) (Table 7) | 500 |
| glucose (g/L) | 19.5 |
| Yeast vitamin solution (mL/L) (Table 9) | 12 |
| Yeast trace metals solution (mL/L) (Table 9) | 10 |

Batch medium was prepared by combining 2 × batch base with tap water in a 2 L bioreactor, autoclaving the unit, and in a sterile hood bringing the volume of the solution to 90% of final by adding concentrated filter-sterilized stock solutions of sugar, vitamins, and trace metals. The remaining 10% of starting volume was from the seed culture.

TABLE 9
Vitamin and trace metals stock solutions

| | Yeast vitamin solution |
|---|---|
| Component | (g/L) ^{a)} |
| Biotin | 0.05 |
| calcium pantothenate | 1 |
| nicotinic acid | 1 |
| Myoinositol | 25 |
| thiamine HCl | 1 |
| pyridoxol HCl | 1 |
| p-aminobenzoic acid | 0.2 |
| | Yeast trace metals |
| Component | solution (g/L) ^{b)} |
| ZnSO₄*7H₂O | 5.75 |
| MnCl₂*4H₂O | 0.32 |
| CuSO₄ anhydrous | 0.32 |
| CoCl₂*6H₂0 | 0.47 |
| Na₂MoO₄*2H₂O | 0.48 |
| CaCl₂*2H₂O | 2.9 |
| FeSO₄*7H₂O | 2.8 |
| 0.5M EDTA | 80 (mL/L) |

^{a)} Biotin was first dissolved in 10 mL of 5M NaOH, and then added to DI water (750 mL/L). The pH was adjusted to 6.5 using 5M NaOH or HCl, and again adjusted after the addition of each vitamin. After all vitamins were dissolved, the solution was brought to final volume with DI water, and filter sterilized. The bottle was covered in aluminum foil and stored at 4° C.
^{b)} EDTA was first added to DI water (750 mL/L) before the ZnSO₄ was dissolved. The pH was adjusted to 6.0 using 5M NaOH, and again adjusted after the addition of each metal. After all metals were dissolved, the pH was adjusted to 4.0 using 5M HCl, and the solution was brought to the final volume with DI water, and filter sterilized. The bottle was covered in aluminum foil and stored at 4º C.

The pH of the fermentation was controlled automatically and maintained at pHI 5 with the addition of 10 NH₄OH. Temperature was maintained at 30° C. Airflow was supplied at a rate of 1 LPM. Dissolved oxygen was maintained at 40% with an agitation cascade followed by oxygen enrichment. Foam was controlled with Biospumex antifoam 200 K.

The bioreactor culture was allowed to grow until glucose in the batch medium was depleted, at which point, an exponential glucose feed was initiated for which glucose feed medium (Table 10) was pumped into the bioreactor at the rate defined by the following equations:

$$F = V {μ}_{set} {S}_{B} {e}^{{μ}_{set} ( t - {t}_{o} )} \\ V = {V}_{0} + {V}_{feed}$$

F is the substrate mass flow rate (g/hr), V is the liquid volume in the bioreactor at a given time (L), S_{B} is the concentration of substrate in the batch media (20 g/L), μₛₑₜ is the specific feed rate (0.087 hr⁻¹), t is the batch age (hr), t₀ is the batch age when the feed was initiated (hr), V₀ is the initial volume in the bioreactor, and V_{feed} is the total volume of feed added to the bioreactor at a given time (L). The exponential feed phase continued until the ratio of F/V reached a preset maximum feed rate (Table 11). After reaching this maximum, the ratio of F/V was maintained constant for the remainder of the process at a preset stationary feed rate (Table 11).

TABLE 10
Bioreactor feed media

| | | |
|---|---|---|
| | Glucose Feed | Mixed Feed |
| Component | Medium ^{a)} | Medium ^{b)} |

Base Medium

| | | |
|---|---|---|
| glucose monohydrate (g/L) ^{a)} | 650 | 425 |
| KH₂PO₄ (g/L) | 9 | 9 |
| MgSO₄*7H2O (g/L) | 5.12 | 5.12 |
| K₂SO₄ (g/L) | 3.5 | 3.5 |
| Na₂SO₄ (g/L) | 0.28 | 0.28 |

Supplmentary Components

| | | |
|---|---|---|
| Yeast vitamin solution (mL/L) (Table 9) | 12 | 12 |
| Yeast trace metals solution (mL/L) (Table 9) | 10 | 10 |
| 95% (v/v) ethanol (mL/L) | 0 | 237 |

^{a)} Glucose feed medium was prepared by mixing glucose monohydrate, KH₂PO₄, MgSO₄*7H2O, K₂SO₄, and Na₂SO₄ in 38° C. tap water, cooling the solution, filter sterilizing, adding the supplementary components (concentrated filter-sterilized stock solutions of trace metals and vitamins) in a sterile hood, and bringing the solution to final volume by adding sterile water.
^{b)} Mixed feed medium was prepared by mixing glucose, KH₂PO₄, MgSO₄*7H2O, K₂SO₄, and Na₂SO₄ in 300 mL of 38° C. tap water, heating the mixture to approximately 100° C. to fully dissolve the sugar and salts, adding water to bring the volume to 750 mL, cooling the solution, filter sterilizing using a 0.2 micron filter, adding first 237 mL of 95% (v/v) ethanol and adding the supplementary components (concentrated filter-sterilized stock solutions of trace metals and vitamins) in a sterile hood, and bringing the solution to the final volume of 1 L by adding sterile water.

Production of amorpha-4,11-diene was induced at an OD₆₀₀ of 50 about 24 hours after inoculation with the addition of 10 g/L galactose to the bioreactor and feed bottle (22.2 mL of a 450 g/L galactose stock solution per liter culture volume). In addition, 0.25 g/L methionine was added to the bioreactor and 1 g/L methionine was added to the feed bottle to repress transcription of the ERG9 gene (10 mL of a 25 g/L methionine stock solution per liter culture volume and 40 mL of a 25 g/L methionine stock solution per liter feed volume), and 10% v/v of autoclaved methyl oleate was added to the bioreactor to capture the amorpha-4,11-diene. (The 450 g/L galactose stock solution was prepared by dissolving the sugar in water with heating, allowing the solution to cool, and filter sterilizing. The 25 g/L methionine stock solution was prepared by dissolving methionine in water, and filter sterilizing the solution.)

Samples were taken at various time points and diluted at a ratio of 1:20 into methanol. Each diluted sample was vortexed for 30 minutes, and culture debris was spun down. Amorpha-4,11-diene titers were determined by transferring 5 to 10 uL of the supernatant to a clean glass vial containing 990 to 995 uL ethyl acetate spiked with trans-caryophyllene as an internal standard. The ethyl acetate samples were analyzed on an Agilent 7890N gas chromatograph equipped with a flame ionization detector (Agilent Technologies Inc., Palo Alto, CA). Compounds in a 1 uL aliquot of each sample were separated using a DB Wax column (Agilent Technologies, Inc., Palo Alto, CA), helium carrier gas, and the following temperature program: 220° C. hold for 3 minutes, increasing temperature at 100° C./minute to a temperature of 260° C. Using this protocol, amorpha-4,11-diene has a retention time of approximately 3.4 minutes. Amporpha-4,11-diene titers were calculated by comparing generated peak areas against a quantitative calibration curve of purified amorpha-4,11-diene in trans-caryophyllene-spiked ethyl acetate.

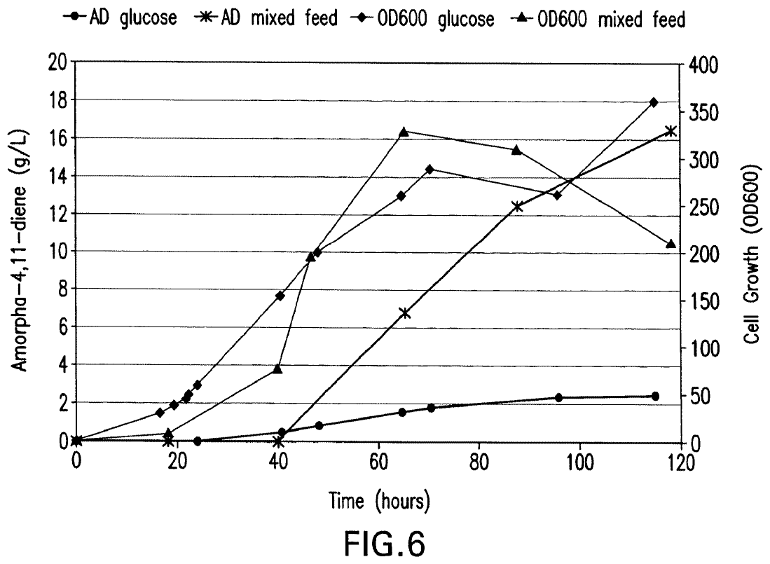

As shown in Table 11 and FIG. 6, strain Y337 produced 2.4 g/L amorpha-4,11-diene (AD) at 114 hours after the start of the fermentation in the glucose only feed process.

TABLE 11
Amorpha-4,11-diene production by strain Y337 using either a glucose
feed or a glucose/ethanol mixed feed

| Glucose | Ethanol | Maximum | Stationary | Maximum | Yield at |
|---|---|---|---|---|---|
| in Feed | in Feed | Feed | Feed | AD | Maximum Titer |
| Medium | Medium | Rate | Rate | Titer | (mg product/g |
| (g/L) | (g/L) | (g/hr/L) ^{a)} | (g/hr/L) ^{a)} | (g/L) | substrate) |
| 545 | 0 | 10 | 10 | 2.4 | 5.4 |
| 340 | 180 | 8.6 | 8.6 | 16.5 | 38.7 |
| 340 | 180 | 8.6 | 4.3 | 12.6 | 50.3 |

^{a)} g/hr/L is g substrate/hr/L bioreactor volume.

### Example 5

This example describes the production of amorpha-4,11-diene by host cells in fed batch, carbon-restricted fermentation with a glucose-ethanol mixed feed.

Y337 seed cultures were prepared and used to inoculate bioreactors as described in Example 4. Fermentations were carried out, and samples were analyzed, essentially as described in Example 4 with the following modifications.

During the early phase of the fermentation, some of the glucose in the batch medium was converted to ethanol. The bioreactor culture was allowed to grow until the glucose and the ethanol in the batch medium was depleted, at which point an exponential feed was initiated for which mixed feed medium (Table 10) was pumped into the bioreactor at the rate defined by the following equations:

$$F = V {μ}_{set} {S}_{B} {e}^{{μ}_{set} ( t - {t}_{o} )} \\ V = {V}_{0} + {V}_{feed}$$

F is the substrate mass flow rate (g/hr), V is the liquid volume in the bioreactor at a given time (L), S_{B} is the concentration of substrate in the batch media (20 g/L), μₛₑₜ is the specific feed rate (0.087 hr⁻¹), t is the batch age (hr), t₀ is the batch age when the feed was initiated (hr), V₀ is the initial volume in the bioreactor, and V_{feed} is the total volume of feed added to the bioreactor at a given time (L). The exponential feed phase continued until the ratio of F/V reached a preset maximum feed rate in units of g substrate/hr/L bioreactor volume (Table 11). After reaching this maximum, the ratio of F/V was maintained constant for the remainder of the process at a preset stationary feed rate (Table 11).

Production of amorpha-4,11-diene was induced at an OD₆₀₀ of 77 about 40 hours after inoculation.

As shown in Table 11 and FIG. 6, strain Y337 produced up to 16.5 g/L amorpha-4,11-diene at 118 hours after the start of the fermentation in the mixed glucose and ethanol feed fermentation.

### Example 6

This example describes the production of amorpha-4,11-diene by host cells in fed-batch, pulse feed fermentation with an ethanol only feed.

Y293 seed cultures were prepared and used to inoculate bioreactors as described in Example 3. Fermentations were carried out, and samples were analyzed, essentially as described in Example 4 with the following modifications:

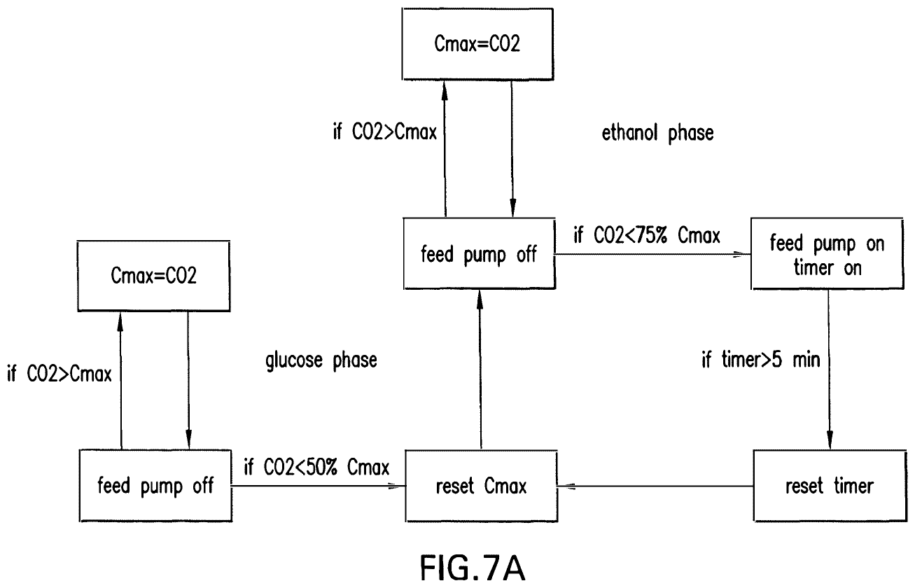

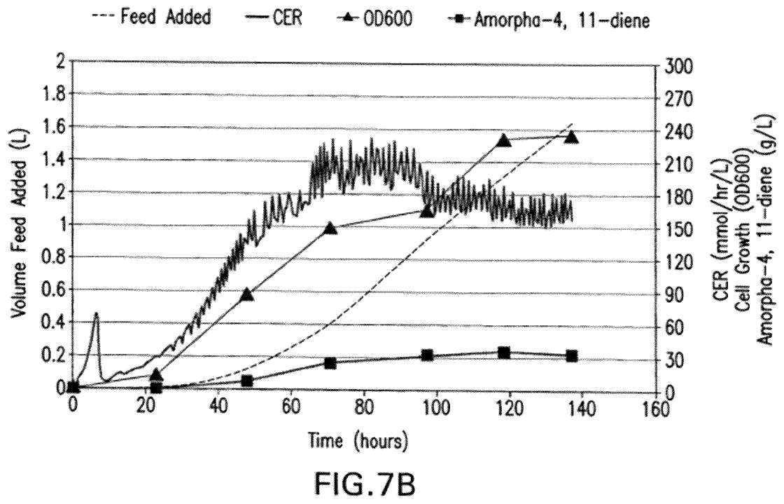

During the early phase of the fermentation, some of the glucose in the batch medium was converted to ethanol. The bioreactor culture was allowed to grow until the glucose and the ethanol in the batch medium was depleted, at which point an ethanol pulse feed was initiated. The rate of the feed was controlled by the percent of CO₂ in the off-gas (the CO₂ evolution rate; CER), which was monitored with an off-gas analyzer and a computer algorithm that assigned a variable (Cₘₐₓ) to the maximum CER which tracked the maximum value of CO₂ percent in off gas. While growing on glucose, the CER evolved rapidly (FIG. 7B). When glucose was depleted from the batch medium, the CER dropped to below 50% of Cₘₐₓ, and the computer algorithm reset Cₘₐₓ to the CO₂ value after the drop. When the ethanol produced from the excess glucose in the batch medium was depleted, the CER dropped a second time. The pulse feed was triggered automatically when the CER fell below 75% of the current Cₘₐₓ. The pump injected 75% (v/v) ethanol into the bioreactor for 5 minutes, delivering approximately 10 g ethanol to the culture. Cₘₐₓ was reset to the value of the percent CO₂ in the off-gas at the time the pump was turned off and then reassign to track the increases in CO₂ evolution, and the pump was reactivated when the CER again fell below 75% of the newly set Cₘₐₓ. The feed algorithm was iterated throughout the fermentation (FIG. 7A), and ensured that the culture was not overfed with ethanol. Because none of the salts, trace metals, vitamins, sugars, or amino acid solutions were soluble in the ethanol feed, concentrated feed components (Table 12) were combined and injected through a septum in the bioreactor head plate once per day according to how much ethanol volume had been delivered since the previous addition of feed components.

TABLE 12
Concentrated feed components

| Component | Amount (mL/L ethanol) |
|---|---|
| glucose (450 g/L) | 24 |
| methionine (25 g/L) | 40 |
| 10 × feed base ^{a)} | 100 |
| Yeast vitamin solution (mL/L) (Table 9) | 12 |
| Yeast trace metals solution (Table 9) | 10 |

^{a)} 90 g/L KH₂PO₄, 51.2 g/L MgSO₄*7H₂O, 35 g/L K₂SO₄, and 2.8 g/L Na₂SO₄

Ten hours after the glucose was depleted from the batch medium, 0.25 g/L methionine was added to the bioreactor through the head plate, and 10% v/v of autoclaved methyl oleate was pumped into the vessel. (Since strain Y293 comprises a disrupted GAL80 gene, galactose was not necessary to induce production of amorpha-4,11-diene.)

As shown in FIG. 7B, strain Y293 produced 36 g/L amorpha-4,11-diene.

### Example 7

This example describes the production of amorpha-4,11-diene by host cells in fed batch, carbon-restricted fermentation with an ethanol only feed.

Y293 seed cultures were prepared and used to inoculate bioreactors containing batch medium (Table 13) as described in Example 3.

TABLE 13
Bioreactor media

| Component | Batch Medium |
|---|---|
| glucose-H₂O (715 g/L) (mL/L) | 19.5 |
| (NH₄)₂SO₄ (g/L) | 15 |
| KH₂PO₄ (g/L) | 26 |
| MgSO₄*7H2O (g/L) | 16.4 |
| K₂SO₄ (g/L) | 7 |
| Na₂SO₄ (g/L) | 0.56 |
| Yeast vitamin solution (mL/L) (Table 9) | 46.3 |
| Yeast trace metals solution (mL/L) (Table 9) | 38.5 |

Fermentations were carried out, and samples were analyzed, essentially as described in Example 4 with the following modifications:

The bioreactor culture was allowed to grow until glucose in the batch medium was depleted, at which point an exponential feed was initiated for which glucose feed medium (Table 10) was pumped into the bioreactor at the rate defined by the following equations:

$$F = V {μ}_{set} {S}_{B} {e}^{{μ}_{set} ( t - {t}_{o} )} \\ V = {V}_{0} + {V}_{feed}$$

F is the substrate mass flow rate (g/hr), V is the liquid volume in the fermentor at a given time (L), S_{B} is the concentration of substrate in the batch media (20 g/L), μₛₑₜ is the specific feed rate (0.087 hr⁻¹), t is the batch age (hr), t₀ is the batch age when the feed was initiated (hr), V₀ is the initial volume in the fermentor, and V_{feed} is the total volume of feed added to the fermentor at a given time (L). The exponential feed continued until the maximum feed rate of 7.1 g/hr/L was reached (OD₆₀₀ of approximately 50). At that point, the feed was switched to an ethanol feed (190 proof), and the feed rate was set to a constant volumetric value of 2.5 g/hr/L for the remainder of the fermentation. With this programmed feed rate, ethanol consumption rates were controlled, and ranged from 0.4 to 1.75 g ethanol/g DCW/day.

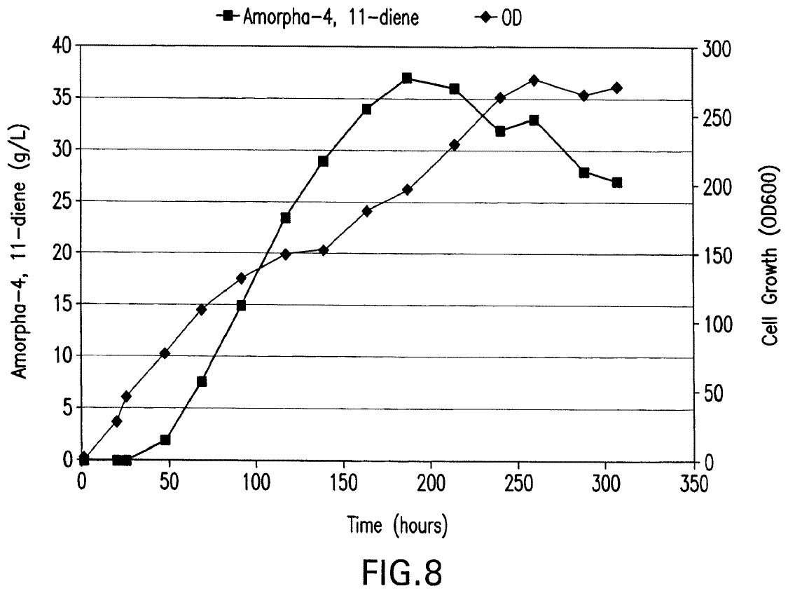

As shown in FIG. 8, strain Y293 produced 37 g/L amorpha-4,11-diene at 187 hours after the start of fermentation.

### Example 8

This example describes the production of farnesene by host cells in fed batch, carbon-restricted fermentation with an ethanol only feed.

Y677 seed cultures were prepared and used to inoculate two bioreactors each containing 630 mL batch medium (Table 14) as described in Example 3. To one of the two bioreactors, 200 mL methyl oleate was added for product capture. Fermentations were carried out, and samples were analyzed, essentially as described in Example 4 with the following modifications:

TABLE 14
Bioreactor media

| Component | Batch Medium |
|---|---|
| Glucose (g/L) | 39.03 |
| (NH₄)₂SO₄ (g/L) | 15 |
| KH₂PO₄ (g/L) | 33.7 |
| MgSO₄*7H2O (g/L) | 20.77 |
| K₂SO₄ (g/L) | 10 |
| Na₂SO₄ (g/L) | 0.8 |
| Yeast vitamin solution (mL/L) (Table 9) | 32.4 |
| Yeast trace metals solution (mL/L) (Table 9) | 27 |

During the early phase of the fermentations, some of the glucose in the batch medium was converted to ethanol. The bioreactor cultures were allowed to grow until the glucose and the ethanol in the batch media were depleted, at which point, an exponential feed was initiated for which pure ethanol (190 proof) was pumped into the bioreactor at the rate defined by the following equations:

$$F = V {μ}_{set} {S}_{B} {e}^{{μ}_{set} ( t - {t}_{o} )} \\ V = {V}_{0} + {V}_{feed}$$

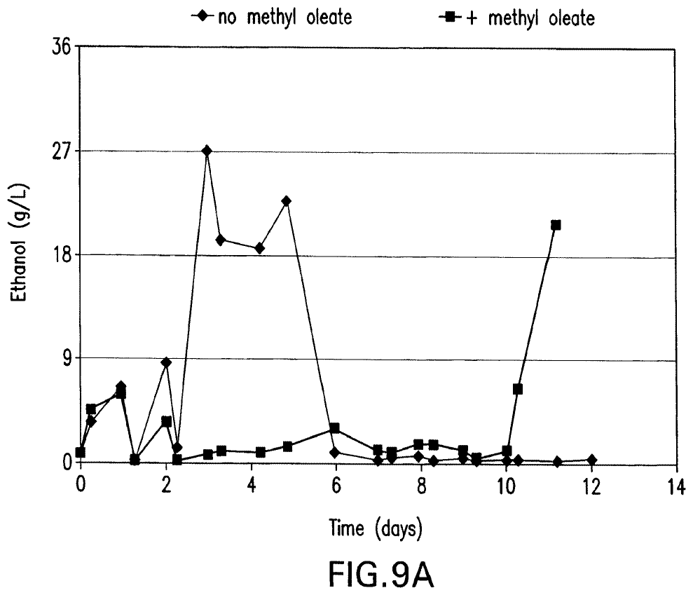

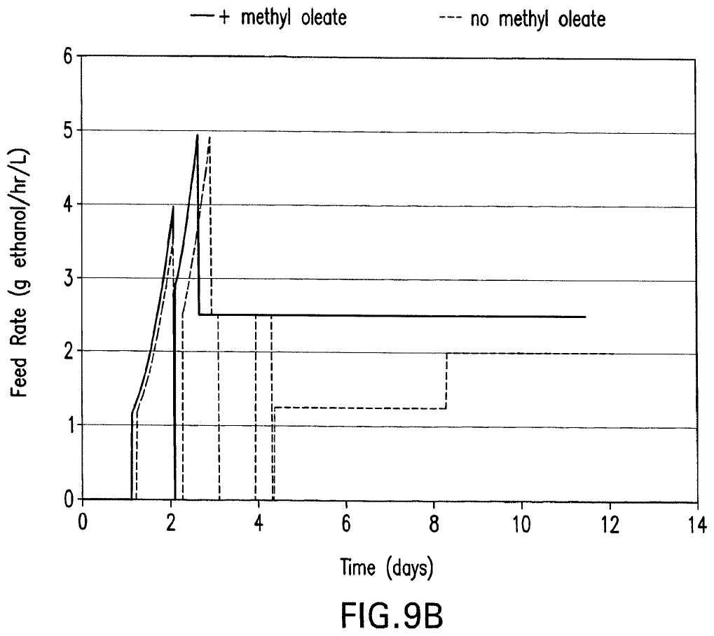

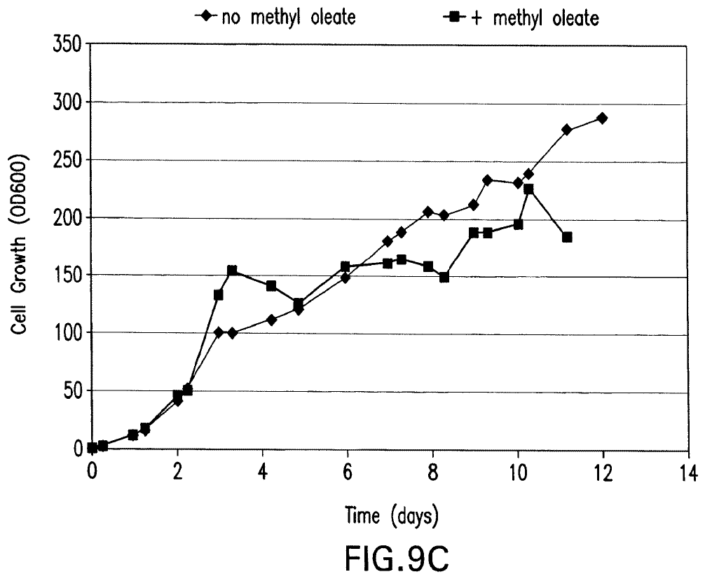

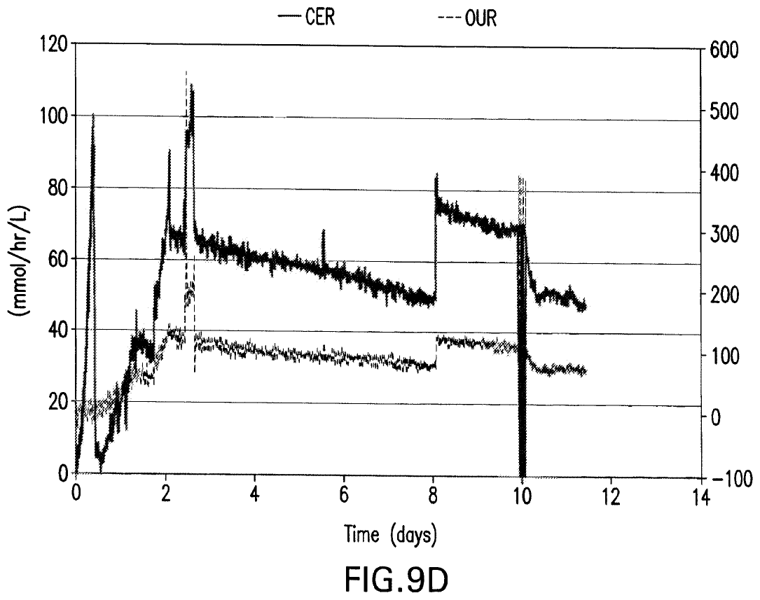

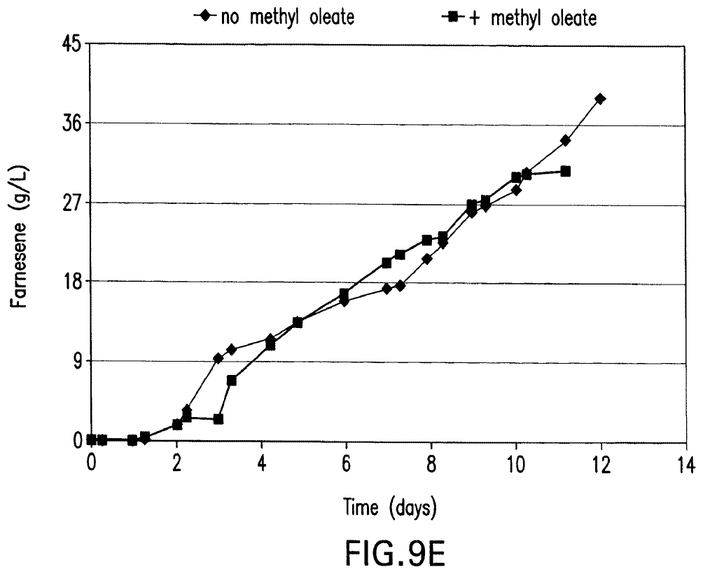

F is the substrate mass flow rate (g/hr), V is the liquid volume in the fermentor at a given time (L), S_{B} is the concentration of substrate in the batch media (39.03 g/L), μₛₑₜ is the specific feed rate (0.058 hr⁻¹), t is the batch age (hr), t₀ is the batch age when the feed was initiated (hr), V₎ is the initial volume in the fermentor (0.7 L), and V_{feed} is the total volume of feed added to the fermentor at a given time (L). The exponential feed phase continued until the ratio of F/V reached a maximum feed rate of 5 g substrate/hr/L bioreactor volume. After reaching this maximum, the ratio of F/V was maintained constant for the remainder of the process at a stationary feed rate of 2.5 g/hr/L. However, as shown in FIG. 9A, the relatively slow rate of ethanol utilization at the beginning of the exponential feed phase resulted in the accumulation of ethanol. This accumulation necessitated manual adjustment of the preset feed rates (FIG. 9B) and an increase in the feed rate doubling time from 12 to 14 hours to maintain a carbon-limited process. Cells grown in the presence of methyl oleate quickly recovered and resumed growth to the preset maximum and stationary feed rates (FIG. 9C). In contrast, the culture that contained no methyl oleate was slower to consume the accumulated ethanol, and thus required a second suspension of the stationary feed followed by a reduction of the stationary feed rate from 2.5 g/hr/L to 1.25 g/hr/L. Overall, strain Y677 had an ethanol consumption rate of 0 to 2.1 g ethanol/g DCW/day in the absence of methyl oleate, and of 0.27-2.9 g ethanol/g DCW/day in the presence of methyl oleate.

The off gas of the bioreactor was led through a condenser to measure oxygen uptake rate (OUR) and CO₂ generation (CER) using an off-gas mass spectrometer. FIG. 9D shows the CER and OUR of strain Y677 in the presence of methyl oleate.

Cell densities and ethanol consumption were monitored by sampling twice a day. At each time point, 1 mL broth samples were taken and diluted 1:1000 in water, and cell density was measured using a spectrophotometer set at 600 nm wavelength.

Levels of ethanol were quantified by HPLC. At each time point, a 1 mL broth sample was taken and diluted 2× in 30 mM sulfuric acid solution (400 μL 30mM sulfuric acid to 400 uL supernatant for a final concentration of 15 mM sulfuric acid, which matched the concentration of the mobile phase solution ). Cells were removed by centrifugation and filtration prior to loading.

Levels of farnesene produced were quantified by GC-FID. At each time point, 100 uL of methyl oleate overlay was taken and diluted 1:40 in ethyl acetate containing 0.001% trans-beta caryophyllene. The mixture was once again diluted 1:100 in ethyl acetate for a final 1:4000 dilution, which fit within the calibration curve for the method. When no methyl oleate was used for product capture, 25 uL culture broth was combined with 975 uL methanol, the mixture was vortexed for five minutes and centrifuged, and finally diluted 1:100 in ethyl acetate containing 0.001% trans-beta caryophyllene before analysis.

As shown in FIG. 9E, in the presence of methyl oleate strain Y677 reached a peak farnesene titer of 30 g/L, and in the absence of methyl oleate it reached a peak farnesene titer of 40 g/L.

### Example 9

This example describes the production of amorpha-4,11-diene and farnesene by host cells in oxygen-restricted fermentation.

Y283 and Y352 seed cultures were prepared and used to inoculate bioreactors containing 800 mL batch medium (Table 15) and 100 mL methyl oleate as described in Example 3.

TABLE 15
Bioreactor media

| Component | Seed Medium | Batch Medium |
|---|---|---|
| glucose (g/L) | 20 | 30 |
| galactose (g/L) | 0 | 5 |
| methionine (g/L) | 0 | 0.25 |
| (NH₄)₂SO₄ (g/L) | 15 | 15 |
| KH₂PO₄ (g/L) | 8 | 8 |
| MgSO₄*7H2O (g/L) | 6.15 | 6.15 |
| Yeast vitamin solution (mL/L) (Table 9) | 12 | 12 |
| Yeast trace metals solution (mL/L) | 10 | 10 |
| (Table 9) | | |
| succinate (0.5M, pH 5.0) (mL/L) (Table 7) | 100 | 0 |

Fermentations were carried out in 2L Sartorius Biostat B plus twins with gas-flow ration controllers. The pH was controlled automatically at pH 5.0 with the addition of 15N NH₄OH and 5N H₂SO₄. Temperature was maintained at 30° C. and Biospumex 200 K brand antifoam was used to control foam. Bioreactors were inoculated between OD500 of 0.6-1 and allowed to grow on 30 g/L glucose.

The off gas of the bioreactor was led through a condenser to measure oxygen uptake rate (OUR) and CO₂ generation (CER) using an off-gas mass spectrometer. The dissolved oxygen (DO) concentration was measured using an O₂ sensor probe (Hamilton, OXYFERM FDA 225, Hamilton Company, Reno, NV) with sensitivity between 10 ppb to saturation.

During the initial phase of the fermentation, the bioreactor culture converted the glucose in the batch medium to biomass and ethanol. When the glucose was consumed (8-14 hours after the start of fermentation depending on the availability of oxygen in the culture) glucose repression of the galactose transport and transcription machinery was alleviated, and gene expression off GAL promoters was induced by the galactose in the batch medium. The batch culture continued growth until ethanol produced in the fermentative stage was depleted, at which point a DO spike marked the end of the cultivation period.

For the aerobic process, clean dry air was sparged into the medium at a rate of 1 LPM. The stir rate was initially set to 400 rpm, and a DO feedback control loop and stir cascade program were used to maintain the DO concentration at 40% (Table 16).

For the micro-aerobic processes, gas flow was reduced to 0.25 LPM to minimize the dilution of gases that reach the off gas analyzer and to increase the sensitivity of the mass spectrometer. The rate of oxygen delivery was varied by using different gas-flow ratios of air to nitrogen (Table 16).

For the strict anaerobic process, 100% nitrogen gas was sparged into the aqueous medium at 0.25 LPM prior to inoculation, and a constant stir rate of 400 rpm was maintained throughout the cultivation (Table 16).

TABLE 16
parameters for fermentations of strain Y283

| | | Controlled | Gas Flow |
|---|---|---|---|
| Process | Conditions | Parameters | Composition |
| Aerobic | 40% DO | starting 400 rpm | 100% air (21% O₂) |
| | | DO feedback control | |
| | | with cascading stir | |
| | | rate | |
| Microaerobic | 0% DO | no DO feedback | 100% air, 0% N2 |
| | | control fixed stir rate | 90% air, 10% N2 |
| | | at 400 rpm | 80% air, 20% N2 |
| | | | 65% air, 35% N2 |
| | | | 50% air, 50% N2 |
| | | | 50% air, 50% N2 |
| | | | 35% air, 65% N2 |
| | | | 20% air, 80% N2 |
| Anaerobic | No air | fixed stir rate at 400 | 0% air, 100% N2 |
| | supplied | rpm | |

Cell densities and ethanol consumption were monitored by sampling twice a day. At each time point, 1 mL broth samples were taken and diluted 1:100 in water, and cell density was measured using a spectrophotometer set at 600 nm wavelength.

Levels of ethanol and farnesene produced were quantified as described in Example 8 except that the methyl oleaste sample was diluted in ethyl acetate to a final 1:400 dilution instead of 1:4000 dilution.

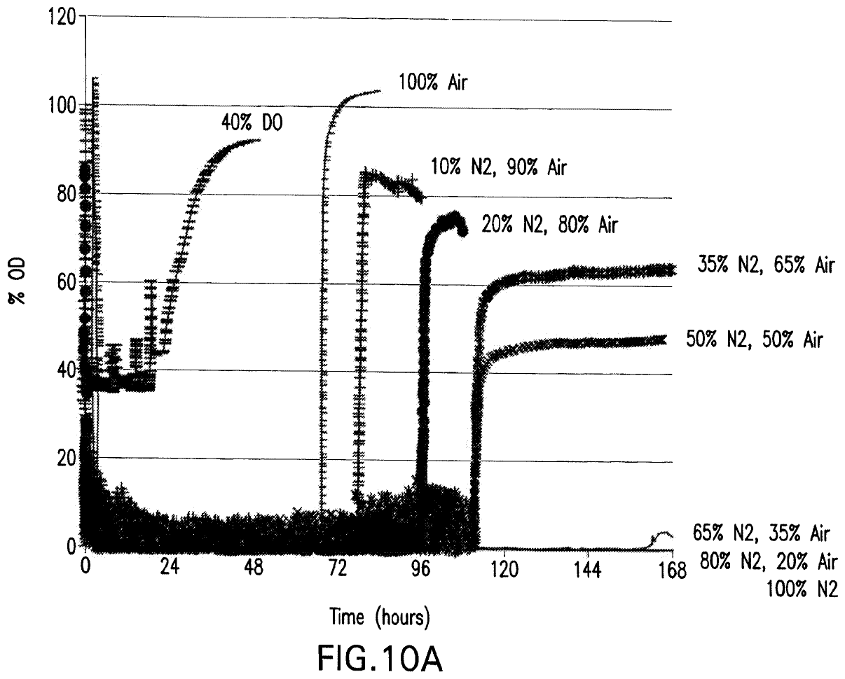

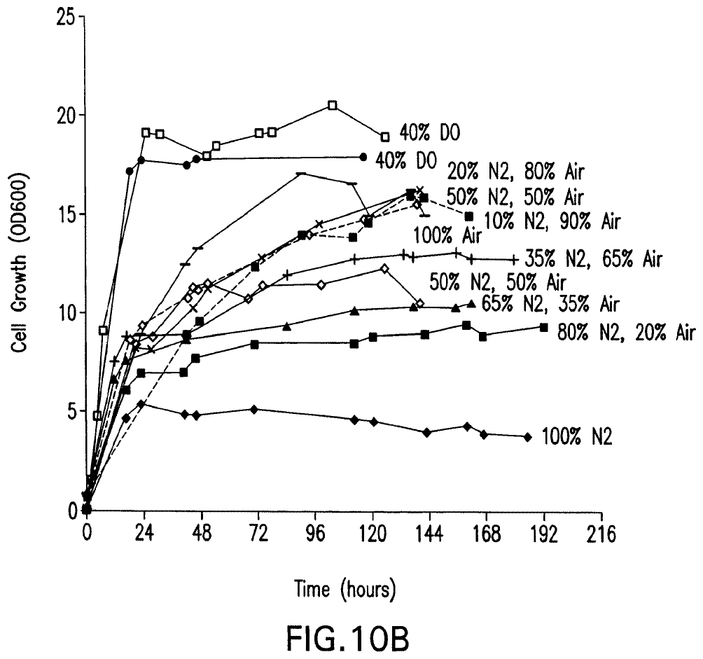

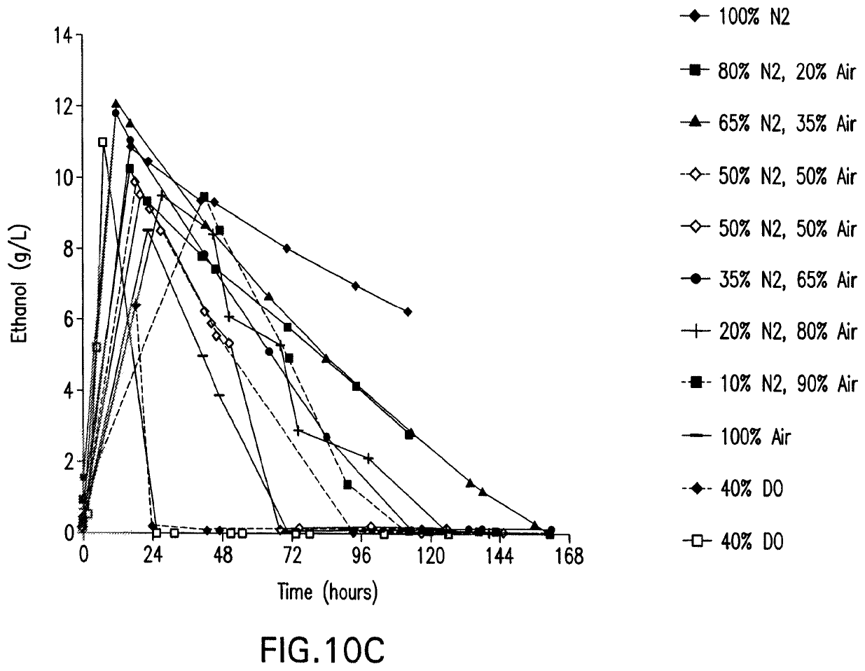

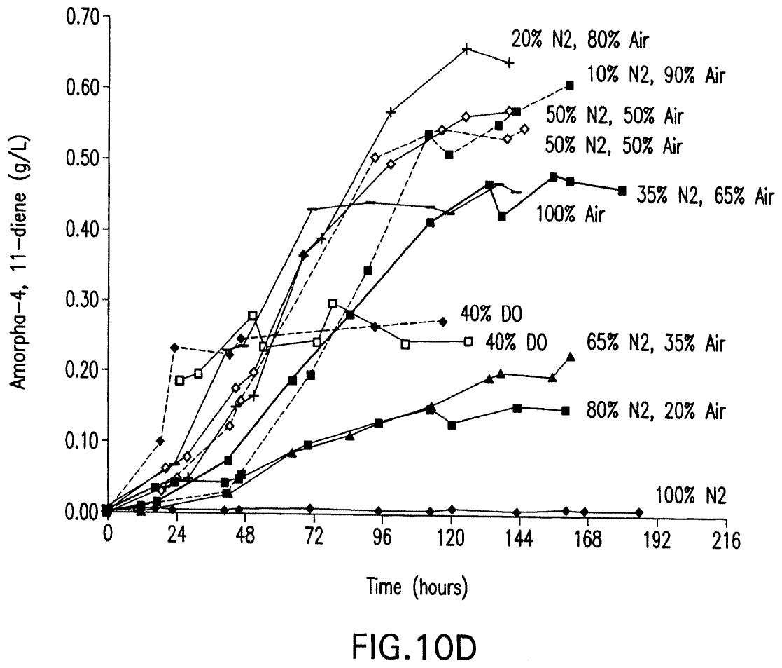

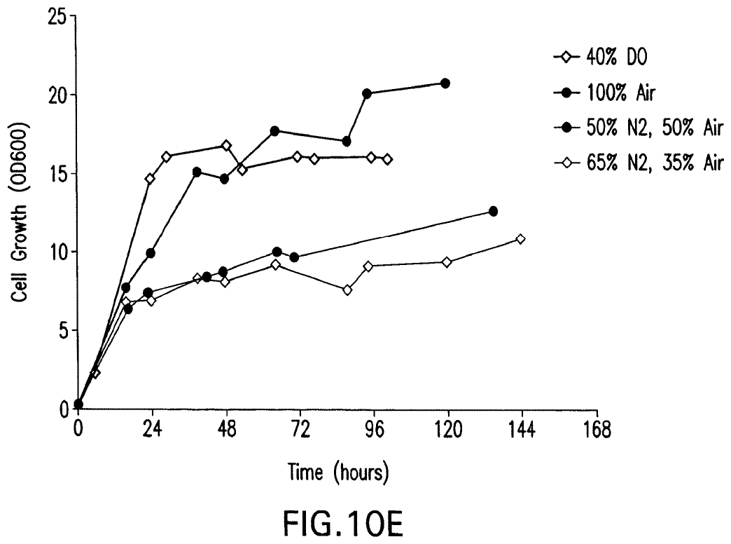

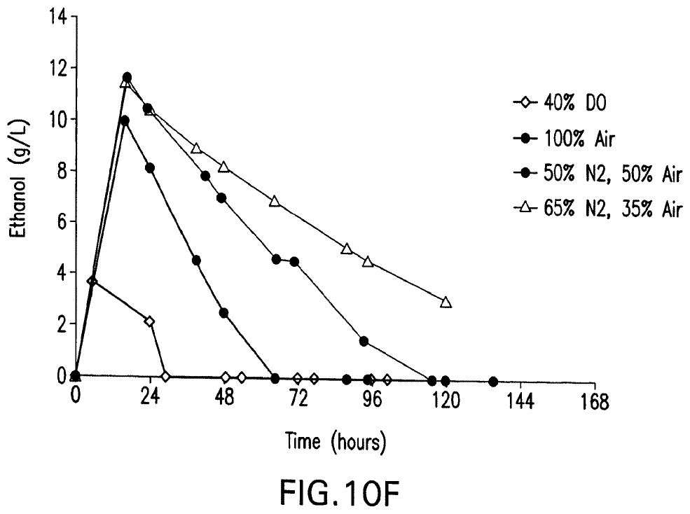

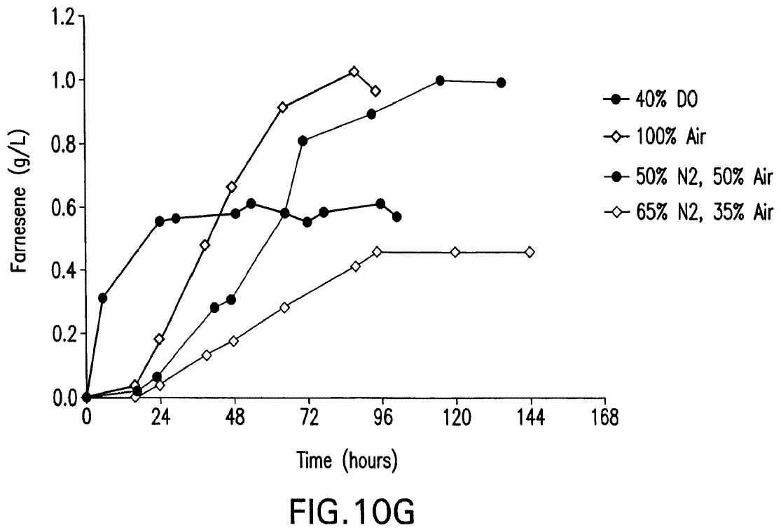

FIG. 10A shows the DO concentrations in the various fermentations of host strain Y283. As shown in FIGS. 10B and 10C, in strain Y283 increased oxygen availability in the culture lead to increased cell growth, increased rate of glucose conversion to ethanol, and increased rate of depletion of ethanol from the medium. Although growth, product formation, and ethanol consumption by strain Y283 were greatest in the fully aerated cultures (DO of 40%), they plateaued after 24 hours. As shown in Table 17, the per cell ethanol consumption rate for all microaerobic processes was between 0.40-0.72 g ethanol/g DCW/day. As shown in FIG. 10D, the best yield of amorpha-4,11-diene relative to carbon input was observed at 80% air and 20% nitrogen.

TABLE 17
Specific ethanol utilization rate (EUR) for microaerobic fermentations

| | Y283 EUR | Y352 EUR |
|---|---|---|
| Gas Ratio | (g ethanol/g DCW/day) | (g ethanol/g DCW/day) |
| 100% N2 | 0.42 | |
| 80% N2 | 0.40 | |
| 65% N2 | 0.42 | 0.42 |
| 50% N2 | 0.65 | 0.69 |
| 50% N2 | 0.58 | |
| 35% N2 | 0.54 | |
| 20% N2 | 0.57 | |
| 10% N2 | 0.60 | |
| 0% N2 | 0.72 | 0.88 |

EUR was calculated from peak measured ethanol to lowest measured ethanol for the fermentation.

As shown in FIGS. 10E and 10F, in strain Y352 increased oxygen availability in the culture lead to increased cell growth, increased rate of glucose conversion to ethanol, and increased rate of depletion of ethanol from the medium. As shown in Table 17, the per cell ethanol consumption rate for the two microaerobic processes tested was between 0.42-0.88 g ethanol/g DCW/day. As shown in FIG. 10G, although slightly higher yield of farnesene on carbon input was observed at 100% air, production continued over a longer period of time in the microaerobic cultures.

### Example 10

This example describes the production of amorpha-4,11-diene by host cells in shake flask cultures with carbon and phosphate restriction.

A stock amyloglucosidase (glucoamylase) enzyme solution was prepared by dissolving solid amyloglucosidase (Sigma A7420-100 MG) in 0.5 M succinate buffer (pH 5.0) to a final enzyme concentration of 100 U/mL, and filter sterilizing the solution.

A Y337 seed culture was prepared by inoculating 1 mL frozen Y337 cells into a 250 mL baffled flask containing 50 mL of phosphate-restricted seed medium (Table 18). The seed culture was grown overnight at 30° C. and 200 rpm.

TABLE 18
Phosphate-restricted shake flask culture media

| | Seed Medium | Production |
|---|---|---|
| Component | (mL/L) | Medium (mL/L) |
| tap water | 350 | 250 |
| 2 × batch base ^{a)} | 500 | 500 (no KH₂PO₄) |
| Yeast vitamin solution (Table 9) | 12 | 12 |
| Yeast trace metals solution (Table 9) | 10 | 10 |
| succinate (0.5M, pH 5.0) (Table 7) | 100 | 100 |
| glucose-H₂O (715 g/L) (Table 7) | 30 | 0 |
| Maltrin M-150 (500 g/L) | 0 | 100 |
| galactose (250 g/L) | 0 | 20 |
| methionine (25 g/L) | 0 | 10 |

^{a)} 1 g/L KH₂PO₄, 30 g/L (NH₄) ₂SO₄, and 12.3 g/L MgSO₄*7H₂O (note: no heating while mixing)

The Y337 seed culture was used to inoculate several 250 mL baffled shake flasks to a starting OD₆₀₀ of 0.05. Production flasks contained 40 mL of phosphate-restricted production medium (Table 18). KH₂PO₄ was added to each flask from a 100 g/L filter-sterilized stock solution to final concentrations of 0.1, 0.25, 0.5, 0.8, 2, and 8 g/L. Prior to inoculation, 80 μL of freshly thawed 100 U/mL amyloglucosidase filter-sterilized stock solution was added to each flask (final concentration of 0.2 U/mL). Production flasks were incubated at 30° C. and 200 rpm for up to 3 days. Over the course of the culture period, glucose was released by glucoamylase at the constant rate of approximately 20 mg/hour.

Amorpha-4,11-diene titers were determined by transferring 2 to 10 μL of the methyl oleate overlay to a clean glass vial containing 500 μL ethyl acetate spiked with beta-or trans-caryophyllene as an internal standard, and analyzing the ethyl acetate samples as described in Example 4.

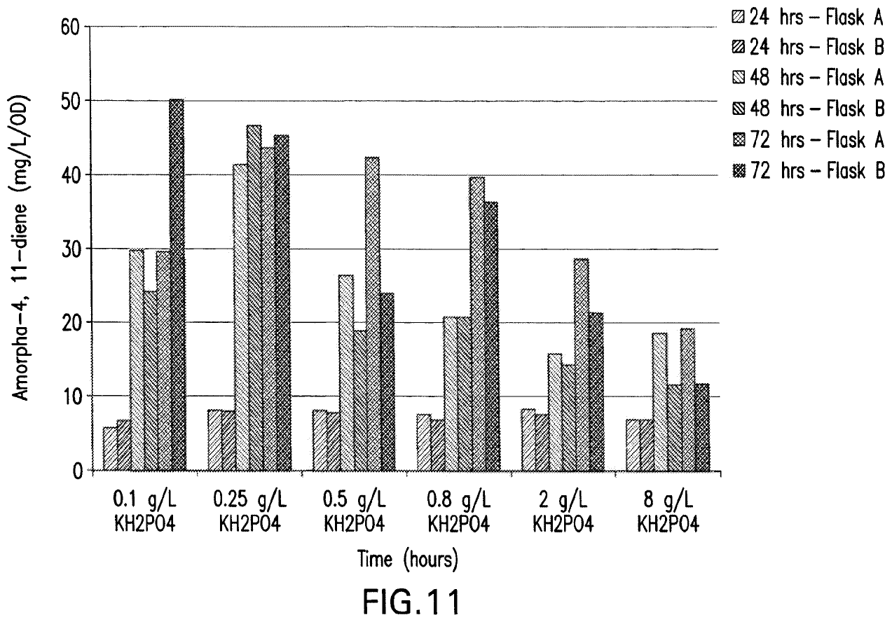

As shown in FIG. 11, overall amorpha-4,11-diene titers were comparable at all phosphate concentrations tested except the lowest (0.1 g/L), but cell growth was limited at lower phosphate concentrations, translating into increased per cell production of amorpha-4,11-diene at lower phosphate concentrations.

### Example 11

This example describes the production of amorpha-4,11-diene by host cells in fed batch, carbon-restricted fermentation with phosphate restriction and a glucose feed.

Y337 seed cultures were prepared and used to inoculate bioreactors containing phosphate-restricted batch medium (Table 19) as described in Example 3. Fermentations were carried out, and samples were analyzed, essentially as described in Example 4 with the following modifications.

The bioreactor culture was allowed to grow until glucose in the batch medium was depleted, at which point, an exponential feed was initiated for which phosphate-restricted glucose feed medium (Table 19) was pumped into the bioreactors at the rate defined by the following equations:

$$F = V {μ}_{set} {S}_{B} {e}^{{μ}_{set} ( t - {t}_{o} )} \\ V = {V}_{0} + {V}_{feed}$$

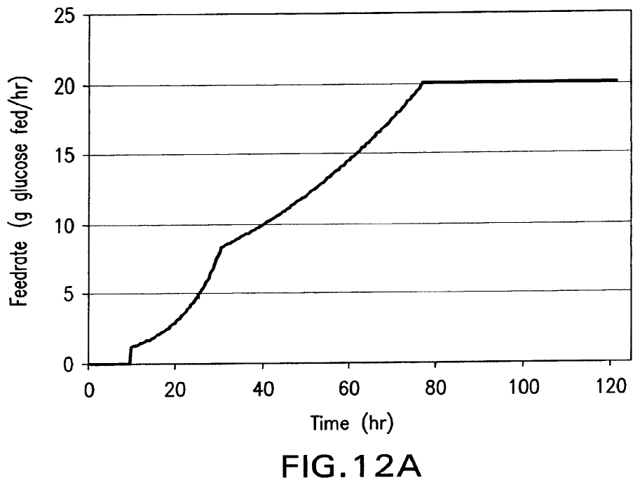

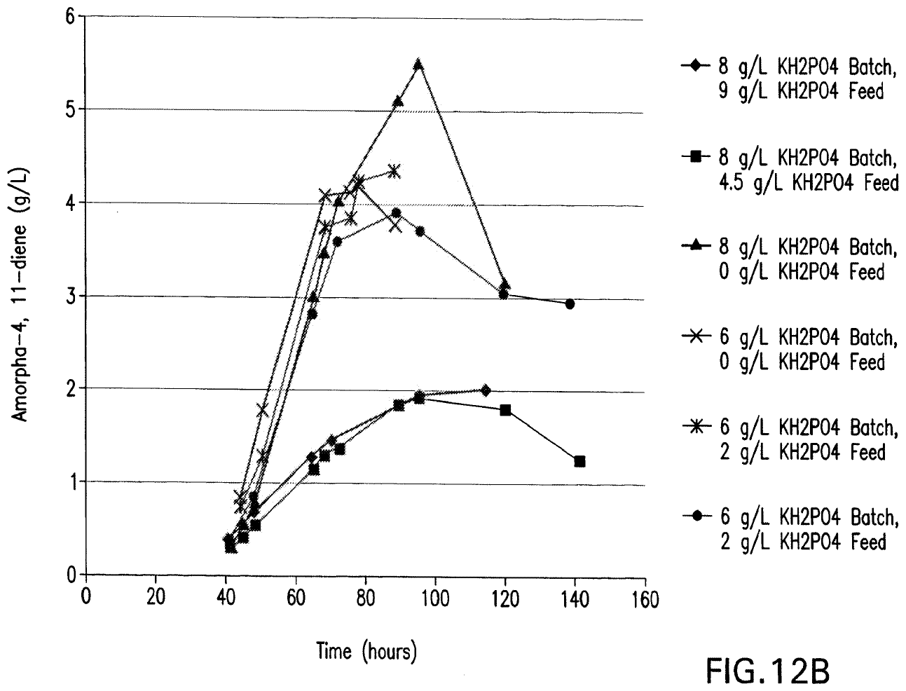

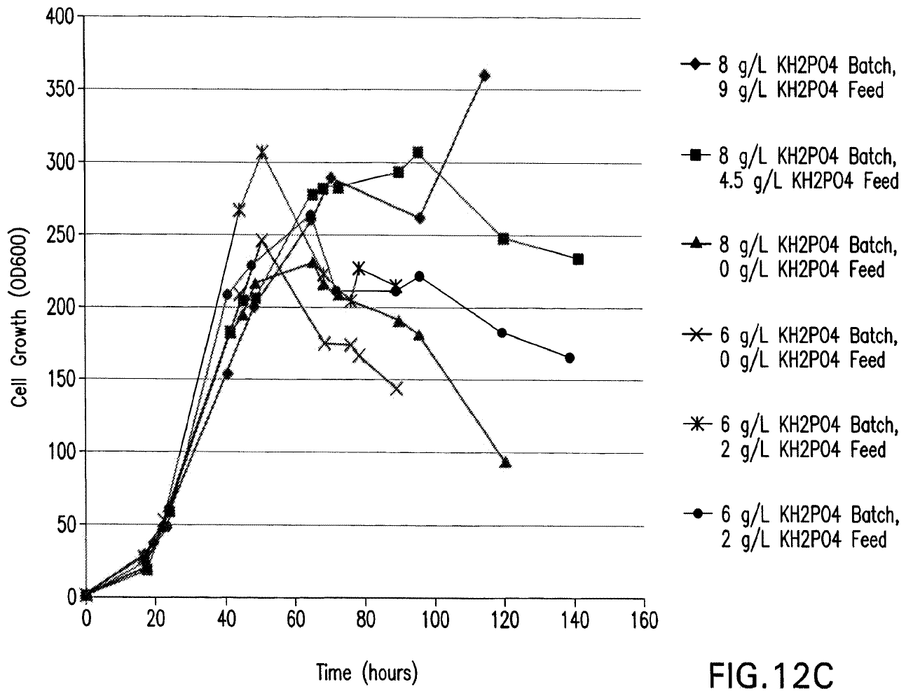

F is the substrate mass flow rate (g/hr), V is the liquid volume in the bioreactor at a given time (L), S_{B} is the concentration of substrate in the batch medium (19.5 g/L), μₛₑₜ is the specific feed rate (0.087 hr⁻¹), t is the batch age (hr), t₀ is the batch age when the feed was initiated (hr), V₀ is the initial volume in the bioreactor, and V_{feed} is the total volume of feed added to the bioreactor at a given time (L). The exponential feed continued until the ratio of F/V reached a preset maximum feed rate (Table 20). After reaching this maximum feed rate, the ratio of F/V was maintained constant for the remainder of the process at a preset stationary feed rate. However, because the volume (V) continued to increase as more feed was added to the bioreactor, the substrate mass flow rate (F) continued to increase until the volume reached the maximum working volume of the bioreactor (approximately 3 times the starting volume). For the rest of the process, the bioreactor volume was held constant by removing cell broth continuously from the reactor, and the substrate mass flow rate (F) was held constant. FIG. 12A shows the glucose feed rate profile of the fermentation.

TABLE 19
Phosphate-restricted bioreactor media

| | | | Glucose | Mixed |
|---|---|---|---|---|
| | Seed | Batch | Feed | Feed |
| Component | Medium ^{a)} | Medium ^{b)} | Medium ^{c)} | Medium ^{d)} |
| glucose (g/L) | 20 | 19.5 | 578 | 425 |
| (NH₄)₂SO₄ (g/L) | 15 | 15 | 0 | 0 |
| KH₂PO₄ (g/L) | 1 | See Tables | See Table | See Table |
| | | 20 and 21 | 20 | 21 |
| MgSO₄*7H2O (g/L) | 6.15 | 6.15 | 5.12 | 5.12 |
| K₂SO₄ (g/L) | 0 | 0 | 3.5 | 3.5 |
| Na₂SO₄ (g/L) | 0 | 0 | 0.28 | 0.28 |
| Yeast vitamin solution | 12 | 12 | 12 | 12 |
| (mL/L) (Table 9) | | | | |
| Yeast trace metals | 10 | 10 | 10 | 10 |
| solution (mL/L) (Table | | | | |
| 9) | | | | |
| succinate (0.5M, pH | 100 | 0 | 0 | 0 |
| 5.0) (mL/L) (Table 7) | | | | |
| 95% (v/v) ethanol | 0 | 0 | 0 | 237 |
| (mL/L) | | | | |

Production of amorpha-4,11-diene was induced at an OD₆₀₀ of approximately 50.

As shown in Table 20 and FIG. 12B, supplying 8 g/L KH₂PO₄ in the batch medium and no phosphate in the feed medium showed the best amorpha-4,11-diene production at 5.52 g/L. Under these conditions, phosphate in the batch medium was consumed by 40 hours, and cell growth was consequently restricted (i.e., less carbon went to biomass and more carbon went to production of amorpha-4,11-diene) (FIG. 12C).

TABLE 20
Amorpha-4, 11-diene production by strain Y337 with glucose feeds and phosphate restriction

| Batch | Feed | Maximum | Stationary | | | |
|---|---|---|---|---|---|---|
| KH₂PO₄ | KH₂PO₄ | Feed Rate | Feed Rate | Time to Max | Maximum | Maximum |
| (g/L) | (g/L) | (g/h/L) ^{a)} | (g/h/L) ^{a)} | Titer (hr) | OD | Titer (g/L) |
| 8 | 9 | 10 | 10 | 114.86 | 360 | 2 |
| 8 | 4.5 | 10 | 10 | 95.62 | 307 | 1.92 |
| 8 | 0 | 10 | 10 | 95.66 | 231 | 5.52 |
| 6 | 0 | 10 | 10 | 78.30 | 246 | 4.2 |
| 6 | 2 | 10 | 10 | 88.98 | 307 | 4.36 |
| 6 | 2 | 10 | 10 | 89.21 | 263 | 3.91 |
| 6 | 2 | 10 | 5 | 119.73 | 274 | 2.98 |

^{a)} g/hr/L is g substrate/hr/L bioreactor volume.

### Example 12

This example describes the production of amorpha-4,11-diene by host cells in fed batch, carbon-restricted fermentation with phosphate restriction and a mixed glucose/ethanol feed.

Y337 seed cultures were prepared and used to inoculate bioreactors containing phosphate-restricted batch medium (Table 19) as described in Example 3. Fermentations were carried out, and samples were analyzed, essentially as described in Example 4 with the following modifications.

During the early phase of the fermentation, some of the glucose in the batch medium was converted to ethanol. The bioreactor culture was allowed to grow until the glucose and the ethanol in the batch medium was depleted, at which point, an exponential feed was initiated for which phosphate-restricted mixed feed medium (Table 19) was pumped into the bioreactor at the rate defined by the following equations:

$$F = V {μ}_{set} {S}_{B} {e}^{{μ}_{set} ( t - {t}_{o} )} \\ V = {V}_{0} + {V}_{feed}$$

F is the substrate mass flow rate (g/hr), V is the liquid volume in the bioreactor at a given time (L), S_{B} is the concentration of substrate in the batch media (20 g/L), User is the specific feed rate (0.087 hr⁻¹), t is the batch age (hr), t₀ is the batch age when the feed was initiated (hr), V₀ is the initial volume in the bioreactor, and V_{feed} is the total volume of feed added to the bioreactor at a given time (L). The exponential feed phase continued until the ratio of F/V reached a preset maximum feed rate in units of g substrate/hr/L bioreactor volume (Table 21). After reaching this maximum, the ratio of F/V was maintained constant for the remainder of the process at a preset stationary feed rate (Table 21).

Production of amorpha-4,11-diene was induced at an OD₆₀₀ of approximately 50.

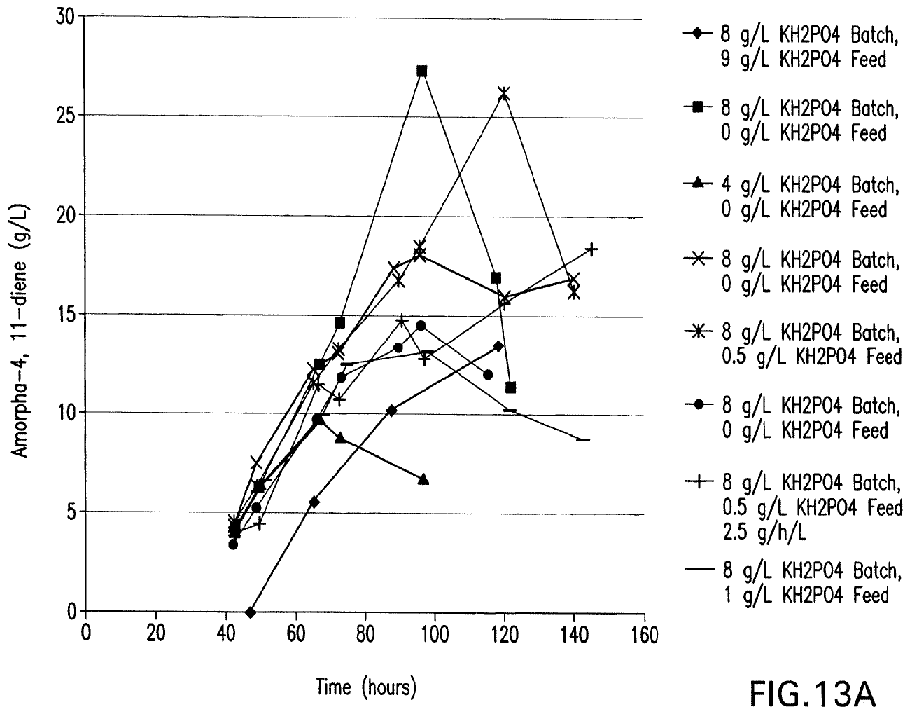

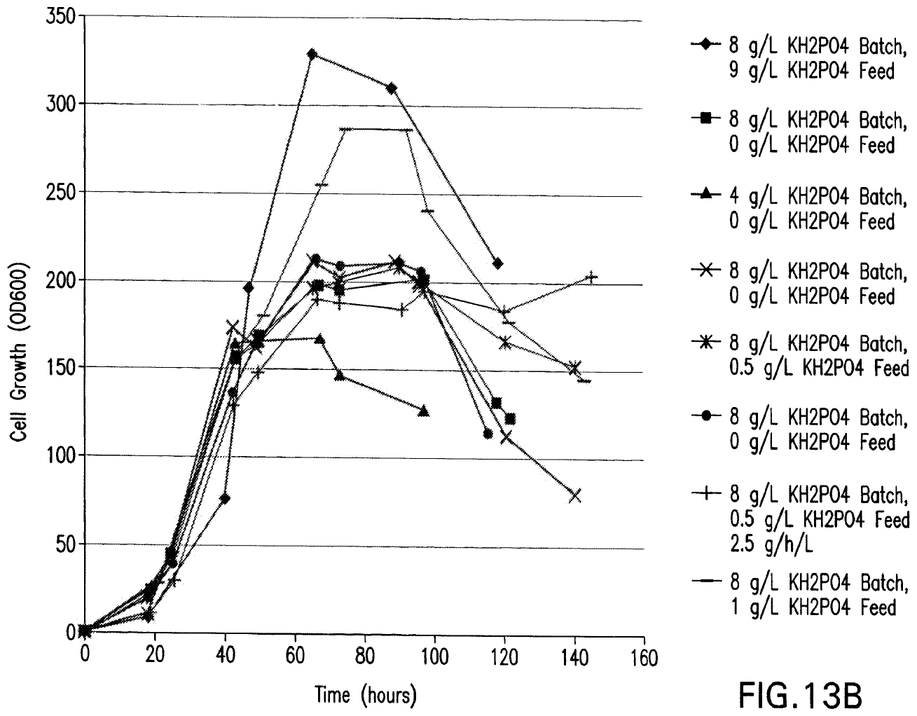

As shown in Table 21 and FIG. 13A, supplying 8 g/L KH₂PO₄ in the batch medium and 0 to 0.5 g/L KH₂PO₄ in the feed medium showed the best amorpha-4,11-diene production at over 26 to 27 g/L. Under these conditions, phosphate in the batch medium was consumed by 40 hours, and cell growth was consequently restricted (i.e., less carbon went to biomass and more carbon went to production of amorpha-4,11-diene) (FIG. 13B). Compared to 0 g/L KH₂PO₄ in the feed medium, 0.5 g/L KH₂PO₄ in the feed medium allowed cell growth and amorpha-4,11-diene production to continue for an additional 24 hours.

TABLE 21
Amorpha-4, 11-diene production by strain Y337 with mixed feeds and phosphate restriction

| Batch | Feed | Maximum | Stationary Feed | Time to | | Maximum |
|---|---|---|---|---|---|---|
| KH₂PO₄ | KH₂PO₄ | Feed Rate | Rate | Maximum | Maximum | Titer |
| (g/L) | (g/L) | (g/h/L) ^{a)} | (g/h/L) ^{a)} | Titer (hr) | OD | (g/L) |
| 8 | 9 | 8.6 | 8.6 | 118.17 | 329 | 12.69 |
| 8 | 9 | 8.6 | 4.3 | 94.85 | 205 | 10.31 |
| 8 | 0 | 8.6 | 8.6 | 96.83 | 201 | 27.36 |
| 4 | 0 | 8.6 | 8.6 | 67.17 | 168 | 9.68 |
| 8 | 0 | 8.6 | 4.3 | 120.20 | 209 | 16.27 |
| 4 | 0 | 8.6 | 4.3 | 120.20 | 181 | 17.94 |
| 8 | 0 | 8.6 | 8.6 | 95.93 | 212 | 18.07 |
| 8 | 0.5 | 8.6 | 8.6 | 120.33 | 209 | 26.23 |
| 8 | 0 | 10 | 10 | 96.13 | 213 | 14.55 |
| 8 | 0.5 | 10 | 10; dropped to | 145.16 | 204 | 18.38 |
| | | | 2.5 at 67 hrs | | | |
| 8 | 1 | 10 | 10 | 97.69 | 287 | 13.15 |

^{a)} g/hr/L is g substrate/hr/L bioreactor volume.

### Example 13

This example describes methods for generating Escherichia coli host strains that harbor heterologous nucleotide sequences encoding enzymes including enzymes of the MEV pathway and terpene synthases integrated in their genomes.

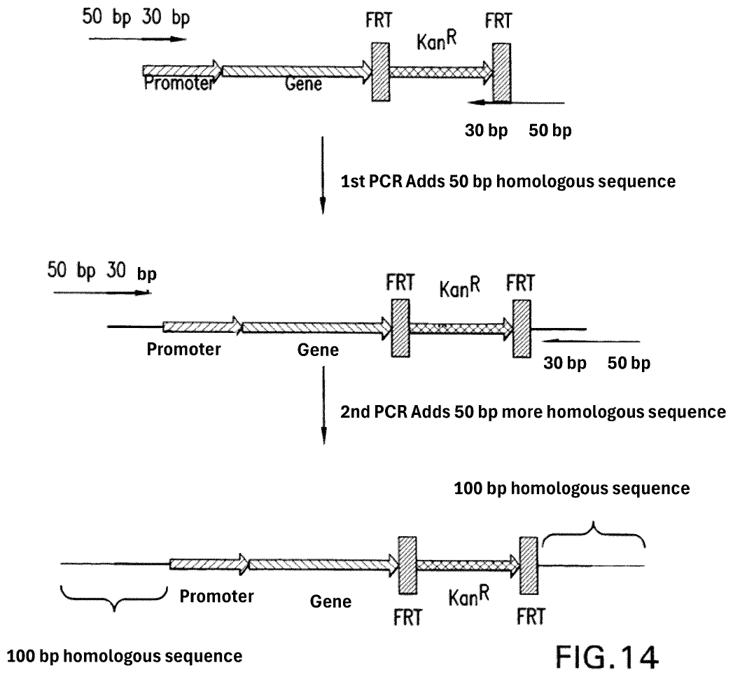

Genomic integrations were carried out using a variation of the procedure outlined by Datsenko & Wanner ((2000) Proc. Natl. Acad. Sci. USA 97:6640-6645). The method employs plasmids that comprise a T7 promoter-gene of interest-FRT-Kan-FRT cassette. The cassette is flanked on each side by approximately 100 nucleotides that are homologous to the regions flanking the genomic locus targeted for the integration of the cassette. The flanking regions are created by PCR amplifying the cassette using primers that comprise a stretch of approximately 30 nucleotides that is homologous to either the 3′ or the 5′ end of the cassette, and another stretch of approximately 50 nucleotides that is homologous to the regions flanking the genomic locus (FIG. 14). The resulting PCR product is used as the template in a 2^{nd} PCR reaction that adds another 50 nucleotides of flanking sequence homology on either end of the cassette (FIG. 14). The cassette with its flanking sequences is electroporated into electro-competent Escherichia coli cells carrying a plasmid that encodes the Red recombinase protein. Kanamycin (“Kan”) resistant colonies are screened by colony PCR. Positive recombinants are treated with P1-phage, and the integration is transferred to a fresh strain via P1-transduction. The resulting strain is transformed with a plasmid that encodes the FLP recombinase, the activity of which causes the Kan gene to be excised from the cassette, leaving behind the T7 promoter-gene of interest at the targeted genomic locus. The final host strain is cured of the FLP recombinase.

Applying the described method, host strain B1060 was generated by integrating a DNA fragment encoding a β-farnesene synthase (“FS”) into the Lac operon of Escherichia coli strain B1021 (MM294(DE3)(T1R)). To this end, Escherichia coli strain MM294 (ATCC33625) was made DE3 using the DE3 lysogenization kit (Novagen, Darmstadt, Germany), and was made resistant to T1 phage by growing the strain in the presence of excess T1 phage, thus yielding strain B1021. A FRT-Kan-FRT cassette was inserted using a modification of the QuikChange methodology (Geiser et al. (2001) Biotechniques 31:88-92) into expression plasmid pAM454, which encodes the β-farnesene synthase of Artemisia annua (GenBank accession number AY835398), codon-optimized for expression in Escherichia coli, under the control of the T7 promoter, thus yielding expression plasmid pAM617. Because the T7-FS-FRT-Kan-FRT cassette in pAM617 is already flanked by sequences from the mhpR and cynX loci (SEQ ID NO: 70), only one round of PCR amplification was necessary to create 100 nucleotide sequences homologous to the mhpR or the cynX sequences that flank the Lac operon. MM294(DE3) host cells harboring expression plasmid pAM88 (encodes the Red recombinase) were grown at 30° C. in LB medium containing 50 ug/mL carbenicillin and 1 mM arabinose to an OD600 of 0.6. The cells were harvested, rendered electro-competent, and transformed with the PCR product. Colonies were obtained after 2 days of growth at 30° C. on LB agar containing 50 ug/mL kanamycin, and the correct integrant was selected by colony PCR. The integration was transferred to a host strain B1021 (MM294 (DE3XT1R)) via P1-transduction, and the resulting strain was made competent and was transformed with expression plasmid pAM89 (encodes the FLP recombinase). Colonies were obtained after 2 days of growth at 30° C. on LB agar containing 50 ug/mL carbenicillin. One colony was isolated and grown at 42° C. in LB media to lose plasmid pAM89, yielding strain B1060 (MM294(DE3)(T1R) lac::T7-FS).

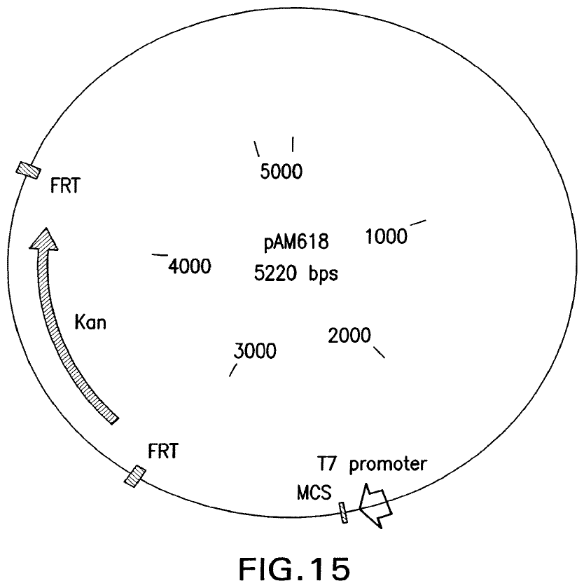

Host strain B1061 was generated by integrating a DNA fragment encoding a mevalonate kinase (“MK”) into the ackpta operon of Escherichia coli strain B1021. To this end, a DNA fragment encoding the mevalonate kinase of Saccharomyces cerevisiae, codon-optimized for expression in Escherichia coli (SEQ ID NO: 71), was inserted into the NdeI BamHI restriction sites of plasmid pAM618. Plasmid pAM618 comprises a T7 promoter followed by a multiple cloning site (MCS) and a FRT-KanR-FRT cassette (SEQ ID NO: 72, FIG. 15). The resulting T7-MK-FRT-Kan-FRT cassette was put through two rounds of PCR amplification as described above to create 100 nucleotide flanking sequences homologous to the ack pta operon. The final PCR product was introduced into Escherichia coli strain B1021 as described above, yielding strain B1061 (MM294(DE3)(T1R) ackpta::T7-MK). The integration was also transferred to host strain B1060, yielding strain B1124 (MM294(DE3)(T1R) lac::T7-FS ackpta::T7-MK).

Host strain B1062 was generated by integrating a DNA fragment encoding a phosphomevalonate kinase (“PMK”) into the poxB locus of Escherichia coli strain B1021. To this end, a DNA fragment encoding the phosphomevalonate kinase of Saccharomyces cerevisiae, codon-optimized for expression in Escherichia coli (SEQ ID NO: 73), was inserted into the NdeI BamHI restriction sites of plasmid pAM618. The resulting T7-PMK-FRT-Kan-FRT cassette was put through two rounds of PCR amplification as described above to create 100 nucleotide flanking sequences homologous to the poxB locus. The final PCR product was introduced into Escherichia coli strain B1021 as described above, yielding strain B1062 (MM294(DE3)(T1R) poxB::T7-PMK).

Host strain B1273 was generated by integrating a DNA fragment encoding a HMG-CoA reductase (“HMGR”) into the ldhA locus of Escherichia coli strain B1021. To this end, a DNA fragment encoding the HMGR of Staphylococcus aureus (mva; GenBank accession number BA000017, REGION: 2688925 . . . 2687648) was inserted into the EcoRI BamHI restriction sites of plasmid pAM618 after treating the EcoRI restriction site with Klenow fragment. The resulting T7-mvaA-FRT-Kan-FRT cassette was put through two rounds of PCR amplification as described above to create 100 nucleotide flanking sequences homologous to the ldhA locus. The final PCR product was introduced into Escherichia coli strain B1021 as described above, yielding strain B1273 (MM294(DE3)(T1R) ldhA::T7-mvaA).

While many specific examples have been provided, the above description is intended to illustrate rather than limit the embodiments provided herein. Many variations of the embodiments will become apparent to those skilled in the art upon review of this specification. The scope of the embodiments should, therefore, be determined not with reference to the above description, but instead should be determined with reference to the appended claims along with their full scope of equivalents.

1.-38. (canceled)

39. A method for producing a heterologous C₅-C₂₀ isoprenoid compound in a yeast host cell, the method comprising:
(a) obtaining a plurality of yeast host cells that are capable of making the heterologous C₅-C₂₀ isoprenoid compound, each said yeast host cell comprising one or more heterologous nucleic acids capable of expressing each enzyme of the MEV pathway or each enzyme of the DXP pathway;
(b) culturing the yeast host cells in media comprising glucose and glucose-converted ethanol and an ethanol-only feed, wherein the culturing includes a period of time where the host cells are carbon-limited to yield a fermentation reaction mixture comprising medium, cells, and the heterologous C₅-C₂₀ isoprenoid compound; and
(c) recovering the heterologous C₅-C₂₀ isoprenoid compound from the medium,
wherein the yeast host cells produce from 10 to 40 grams of the heterologous C₅-C₂₀ isoprenoid compound per liter of fermentation reaction mixture after 4 to 12 days of culturing, and
wherein the heterologous C5-C20 isoprenoid compound is β-farnesene.

40. The method of claim 39, wherein the yeast host cells produce between 30 and 40 grams/liter of β-farnesene.

41. The method of claim 39, wherein the yeast host cells produce greater than about 10 grams/liter of β-farnesene.

42. The method of claim 39, wherein the yeast host cells produce greater than about 20 grams/liter of β-farnesene.

43. The method of claim 39, wherein the yeast host cells are S. cerevisiae.

44. The method of claim 39, wherein each said yeast host cell comprises one or more heterologous nucleic acids capable of expressing an acetyl-CoA acetyltransferase, an HMG-COA synthase, an HMG-CoA reductase, a mevalonate kinase, a phosphomevalonate kinase and a mevalonate pyrophosphate decarboxylase.

45. The method of claim 39, wherein the medium comprises a batch medium and a feed medium.

46. The method of claim 45, wherein the batch medium is supplied with 30 to 35 grams/liter phosphate.

47. The method of claim 39, wherein the yeast host cells are maintained under carbon-limited conditions after the glucose and glucose-converted ethanol are depleted from the batch medium.

48. The method of claim 45, comprising an overall ethanol consumption rate of 0 to 2.1 grams ethanol per gram of dry cell weight per day.

49. The method of claim 45, wherein the batch medium is supplied with 30 to 35 grams/liter phosphate.

50. The method of claim 45, wherein the feed medium is supplied at a maximum feed rate of 5 grams/hour/liter and a stationary feed rate of 1.25 grams/hour/liter to 2.5 grams/hour/liter.

51. The method of claim 45, wherein the batch medium comprises glucose at 39.03 grams/liter and the feed medium is ethanol.

52. The method of claim 51, wherein the feed medium is 95% ethanol.

53. The method of claim 46, wherein the phosphate is supplied to the batch medium as potassium dihydrogen phosphate (KH₂PO₄).
